(19) United States
(12) Patent Application Publication
(10) Pub. No.: US 2011/0287497 A1
(43) Pub. Date: Nov. 24, 2011

(54) ANAEROBIC ORGANISMS IN A PROCESS FOR CONVERTING BIOMASS

(76) Inventors: Mark T. Holtzapple, College Station, TX (US); Cesar B. Granda, College Station, TX (US); Erik Holtzapple, San Diego, CA (US)

(21) Appl. No.: 13/094,589

(22) Filed: Apr. 26, 2011

Related U.S. Application Data
(60) Provisional application No. 61/328,044, filed on Apr. 26, 2010.

(51) Int. Cl. C12P 5/00; C12P 7/62; C12P 7/64

(52) U.S. Cl. 435/134; 435/166; 435/135

(57) ABSTRACT

A method for integrating biological conversion of mixed acids to hydrocarbons, hydrocarbon-like molecules, biofuels, and combinations thereof. The method including introducing fermentation products to at least one microorganism chosen from the group consisting of heterotrophic microorganisms, photo-mixotrophic microorganisms, chemo-autotrophic microorganisms, and combinations thereof.

### CROSS-REFERENCE TO RELATED APPLICATIONS

This application claims the benefit under 35 U.S.C. §119(e) of U.S. Provisional Patent Application No. 61/328,044 filed Apr. 26, 2010 the disclosure of which is incorporated herein by reference.

### STATEMENT REGARDING FEDERALLY SPONSORED RESEARCH OR DEVELOPMENT

Not applicable.

### FIELD OF THE INVENTION

The present invention generally relates to the process of making liquid fuels. More particularly, the present invention relates to integrating anaerobic fermentation and heterotrophic conversion of acid salts to form liquid fuels.

### BACKGROUND

Liquid hydrocarbons derived from oil distillates are currently the predominant energy source for human transportation. Plummeting oil reserve estimates and global price fluctuations have increasingly provided economic impetus for alternative liquid fuel sources. In some countries, investment and research is concentrated in developing biofuels or liquid fuels derived from biological materials. Biofuels may be alcohols, alcohol derived fuels, or liquid hydrocarbons for processing into fuels such as gasoline, diesel, and/or kerosene. Typically, the alcohol or alcohol derived liquid fuels result from the processing of high sugar yield plants, such as corn, switch grass, or sugarcane. While these plants are indefinitely renewable as crops, the arable land needed to grow them is substantial. In certain instances, the growing fuel crops may result in the displacement of food crops. In other instances, food crops such as corn are redirected from processing into food to the processing for fuels.

Alternatively, other approaches to producing biofuels from biomass include algal fuels and microorganism mediated sugar or cellulose hydrolysis. Algae are photosynthetic microorganisms that can accumulate intracellular or produce extracellular hydrocarbon-like molecules. However, culturing algae includes the challenges of contamination, temperature regulation, and obtaining concentrated carbon dioxide. Further the size and expense of operating a viable photoreactor system, including aquatic or marine tanks, conduits, and reservoirs, makes algal biofuels difficult for dry or landlocked areas. With respect to sugar or cellulose hydrolysis, various organisms accumulate intracellularly or produce extracellularly hydrocarbon-like molecules. Sugar is produced by extraction from sugar-bearing plants (e.g. corn, sugar cane), enzymatic hydrolysis of starch, or enzymatic hydrolysis of cellulose. These processes face the technical challenge of cost of enzymes and separating liquid products from undigested or incompletely digested biomass. Further, microorganism mediated hydrolysis of sugars requires similar expenses as algae, mostly related to culturing microorganisms.

Other potential systems and methods for developing biofuels through fermentation and microorganism mediated hydrocarbon production are currently lagging due to the lack of research in these areas and the technological hurdles therein. As the conversion of biomass to produce biofuels independent of food crops or cultivated crops represents a potentially sustainable and renewable source of biofuels and chemical products, there is an industrial demand for finding alternative pathways. Particularly, a system and method to implement a fermentation and microorganism mediated hydrocarbon production.

### BRIEF SUMMARY

The present disclosure relates to a method, comprising: fermenting biomass to fermentation products; converting the fermentation products to hydrocarbon-like molecules biologically; and processing the hydrocarbon-like molecules. The method further comprising processing the hydrocarbon-like molecules to chemical products. And, wherein converting the fermentation products to hydrocarbon-like molecules comprises producing hydrocarbons. The method of fermenting biomass comprises mixed-acid fermentation and producing a dilute solution, wherein the dilute solution comprises acids and salts of acids from biomass solids. Additionally, the method, wherein converting the fermentation products comprises sterilizing the fermentation products, comprising introducing fermentation products to at least one microorganism chosen from the group consisting of heterotrophic microorganisms, chemo-mixotrophic organisms photo-mixotrophic microorganisms, chemo-autotrophic microorganisms, and combinations thereof. The method of the disclosure, wherein introducing fermentation products to heterotrophic organisms to at least one microorganism further comprises mixing an oxidant with the fermentation products, said oxidant chosen from the group consisting of oxygen, nitrates, sulfates, air, and combinations thereof. Further, converting the fermentation products comprises producing extracellular hydrocarbon-like molecules, producing intracellular hydrocarbon-like molecules, or combinations thereof. The hydrocarbon-like products comprise at least one product selected from the group consisting of waxy esters, triacylglycerides, triacylglycerols fatty acid methyl-esters, fatty acid ethyl-esters, poly-hydroxyalkanoates, hydrocarbons, and combinations thereof. Further, according to the disclosure converting the fermentation products to hydrocarbon-like molecules comprises producing hydrocarbons. The method wherein processing hydrocarbon-like molecules comprises isolating the hydrocarbon-like molecules; wherein isolating the hydrocarbon-like molecules comprises lysing microorganisms. The method wherein isolating the hydrocarbon-like molecules comprises separating hydrocarbon-like molecules from other fermentation products. The method wherein processing the hydrocarbon-like molecules comprises producing hydrocarbon liquids, with from about 5 carbons to about 50 carbons. Further the method comprises processing the hydrocarbon-like molecules with at least one method chosen from the group consisting of transesterifying, hydrogenating, decarboxylating, alkylating, isomerizing, polymerizing, oligomerizing, condensing, separating, cleaving, cross-linking, cracking, refining and combinations thereof. The method wherein producing hydrocarbon liquids further comprises producing at least one product chosen from the group consisting of gasoline, aviation gasoline, diesel, biodiesel, kerosene, jet fuel, solvents, lubricants, olefins, alkylolefins, commodity chemicals, and combinations thereof. The method wherein fermenting biomass to produce fermentation products further comprises gasifying undigested fermentation residues; and comprises producing syngas. The method wherein gasifying undigested fermentation residues comprises feeding gasification components to a bioreactor, wherein feeding gasification components to a bioreactor comprises feeding a chemo-autotrophic microorganism. Further according to the disclosure feeding a chemo-autotrophic microorganism comprises introducing syngas from supplemental sources. The method, wherein feeding gasification components to a bioreactor further comprises producing fermentation products for converting to hydrocarbon-like molecules. The method wherein converting fermentation products to hydrocarbon-like molecules further comprises converting supplemental alcohols. The wherein converting fermentation products to hydrocarbon-like molecules further comprises recycling remaining fermentation products to a fermenter. Wherein fermenting biomass to fermentation products further comprises producing ammonia, wherein producing ammonia comprises converting ammonia to ammonium bicarbonate. The method of wherein converting ammonia to ammonium bicarbonate comprises producing a fermentation product salt.

The present disclosure further relates to a hydrocarbon production process comprising fermenting biomass to mixed-acid fermentation products and biologically converting the fermentation products to hydrocarbon-like molecules. The process of the present disclosure further comprising processing the hydrocarbon-like molecules to chemical products. The process wherein converting the fermentation products to hydrocarbon-like molecules comprises producing hydrocarbons. Further, fermenting biomass comprises anaerobic fermentation to a dilute solution of acids and salts of acids. The process comprises separating the dilute solution from biomass solids. The process wherein separating the dilute solution further comprises recycling the biomass solids for further fermenting. The process wherein converting the fermentation products further comprises introducing fermentation products to at least one microorganism chosen from the group consisting of heterotrophic microorganisms, chemo-mixotrophic organisms, photo-mixotrophic microorganisms, chemo-autotrophic microorganisms, and combinations thereof. The process of claim 38, wherein introducing fermentation products to organisms further comprises sterilizing the fermentation products, mixing at least one gas with the fermentation products, said at least one gas selected from the group consisting of hydrogen, oxygen, nitrates, sulfates, air, carbon dioxide, carbon monoxide, and combinations thereof, and mixing at least one supplemental alcohol chosen from the group consisting of methanol, ethanol, glycerol, and combinations thereof. Also, converting the fermentation products comprises producing extracellular hydrocarbon-like molecules. Further, converting the fermentation products comprises producing intracellular hydrocarbon-like molecules. The process wherein hydrocarbon-like products comprise at least one product chosen from the group consisting of waxy esters, triacylglycerides, triacylglycerols fatty acid methyl-esters, fatty acid ethyl-esters, poly-hydroxyalkanoates, hydrocarbons, and combinations thereof. The process wherein converting the fermentation products to hydrocarbon-like molecules comprises producing hydrocarbons. The process wherein processing hydrocarbon-like molecules comprises isolating the hydrocarbon-like molecules from other fermentation products. The process wherein isolating the hydrocarbon-like molecules comprises lysing microorganisms. Further, the process wherein processing the hydrocarbon-like molecules comprises producing hydrocarbon liquids further comprises producing at least one product chosen from the group consisting of gasoline, aviation gasoline, diesel, biodiesel, kerosene, jet fuel, solvents, lubricants, olefins, alkylolefins, commodity chemicals, and combinations thereof. The process, wherein producing hydrocarbon liquids comprises producing hydrocarbons with between about 5 carbons and about 50 carbons and also, wherein producing hydrocarbon liquids further comprises at least one process chosen from the group consisting of transesterifying, hydrogenating, decarboxylating, alkylating, isomerizing, polymerizing, oligomerizing, condensing, separating, cleaving, cross-linking, cracking, refining and combinations thereof. The process wherein fermenting biomass to produce fermentation products further comprises gasifying undigested fermentation residues to syngas. The process wherein gasifying undigested fermentation residues to syngas, further comprises a water-gas shift reaction. Further, according to disclosure, the process wherein gasifying undigested fermentation residues to syngas comprises producing electricity. The process wherein gasifying undigested fermentation residues to syngas further comprises purifying hydrogen and directing the hydrogen for converting fermentation products to hydrocarbon-like molecules or hydrocarbons and wherein purifying hydrogen comprises purifying hydrogen from a supplemental hydrogen source.

A hydrocarbon-fuel production process, comprising fermenting biomass to acid/salt fermentation products, and converting acid/salt fermentation products to hydrocarbon molecules. The process wherein converting the acid/salt fermentation products comprises producing extracellular hydrocarbon-like molecules. The process wherein converting the acid/salt fermentation products comprises producing intracellular hydrocarbon-like molecules. The process further comprising processing the hydrocarbon molecules to produce a hydrocarbon fuel chosen from the group consisting of gasoline, aviation gasoline, diesel, biodiesel, jet fuel, kerosene. The process wherein fermenting biomass to acid/salt fermentation products comprises anaerobic fermenting to a dilute solution and separating solids from the dilute solution. Also, the process wherein converting the fermentation products comprises introducing fermentation products to at least one microorganism chosen from the group consisting of heterotrophic microorganisms, photo-mixotrophic microorganism, chemo-autotrophic microorganisms, and combinations thereof. The process wherein introducing fermentation products to at least one organism further comprises sterilizing the fermentation products, mixing at least one reactant gas with the fermentation products, said gas chosen from the group consisting of hydrogen, oxygen, nitrates, sulfates, air, carbon dioxide, carbon monoxide, light, and combinations thereof, and mixing at least one supplemental alcohol with the fermentation products, said alcohol chosen from the group consisting of methanol, ethanol, glycerol, and combinations thereof. The process wherein converting the fermentation products comprises producing extracellular hydrocarbon-like molecules or producing intracellular hydrocarbon-like molecules and wherein hydrocarbon-like products further comprise at least one product chosen from the group consisting of waxy esters, triacylglycerides, triacylglycerols fatty acid methyl-esters, fatty acid ethyl-esters, poly-hydroxyalkanoates, hydrocarbons, and combinations thereof. The process wherein converting the fermentation products to hydrocarbons comprises biologically producing hydrocarbons and wherein biologically producing hydrocarbons comprises isolating hydrocarbon liquids. The process wherein isolating the hydrocarbon molecules comprises lysing microorganisms to form a hydrocarbon liquid with hydrocarbons with between about 5 carbons and about 50 carbons by a process chosen from the group consisting of transesterifying, hydrogenating, decarboxylating, isomerizing, cleaving, cross-linking, refining, cracking, polymerizing, separating, cleaving, and combinations thereof.

The foregoing has outlined rather broadly the features and technical advantages of the invention in order that the detailed description of the invention that follows may be better understood. The various characteristics described above, as well as other features, will be readily apparent to those skilled in the art upon reading the following detailed description of the preferred embodiments, and by referring to the accompanying drawings.

### BRIEF DESCRIPTION OF THE DRAWINGS

For a more detailed description of certain embodiments of the present invention, reference will now be made to the accompanying figures, wherein:

FIG. 1 is a table illustrating envisioned activity of microorganisms according to an embodiment of the disclosure.

FIG. 2 is a graph of the acid concentration versus conversion efficiency for a fermentor.

FIG. 3 is a block flow diagram for the integration of fermentation and heterotrophic conversion.

FIG. 4 is a process schematic for the integration of fermentation and heterotrophic conversion using the TCA cycle and O₂ as electron acceptor.

FIG. 5 is a process schematic for the integration of fermentation and heterotrophic conversion using the H₂ dehydrogenase pathway and O₂ as electron acceptor.

FIG. 6 is a process schematic for the integration of fermentation and heterotrophic conversion using the H₂ dehydrogenase pathway and nitrate as electron acceptor.

FIG. 7 is a process schematic for the integration of fermentation and heterotrophic conversion using the H₂ dehydrogenase pathway and sulfate as electron acceptor.

FIG. 8 is a process schematic for integration of fermentation and aerobic heterotrophic conversion to hydrocarbon products.

FIG. 9 a process schematic for integration of fermentation and anaerobic heterotrophic conversion to hydrocarbon products via the H₂ dehydrogenase pathway.

FIG. 10 is block flow diagram of the integration of fermentation and chemo-mixotrophic conversion.

FIG. 11 is a process schematic for integration of fermentation and chemo-mixotrophic conversion to hydrocarbon and/or hydrocarbon-like molecules for hydrocarbon products.

FIG. 12 is a process schematic for integration of fermentation and chemo-mixorotrophic conversion to hydrocarbon products.

FIG. 13 is a block flow diagram for integration of fermentation and photo-mixotrophic conversion hydrocarbon and/or hydrocarbon-like molecules for hydrocarbon products.

FIG. 14 is a process schematic for integration of fermentation and photo-mixotrophic conversion to hydrocarbon and/or hydrocarbon-like molecules for hydrocarbon products.

FIG. 15 is another process schematic for integration of fermentation and photo-mixotrophic conversion to hydrocarbon and/or hydrocarbon-like molecules for hydrocarbon products.

FIG. 16 is a process schematic for integration of fermentation and photo-mixotrophic conversion to hydrocarbon products

FIG. 17 is another process schematic for integration of fermentation and photo-mixotrophic conversion to hydrocarbon products.

### DETAILED DESCRIPTION

### Overview

Disclosed herein are systems, apparatuses, and processes related to the production of liquid hydrocarbons or biofuels, by the integration of fermentation and microorganism mediated synthesis. The apparatuses, systems, and processes are generally related to the anaerobic fermentation of biomass to acids and/or acid salts, hereinafter acids/salts, in a fermentation broth. Additionally, the apparatuses, systems, and methods are generally related to the conversion of the acids/salts to hydrocarbons or hydrocarbon-like molecules in subsequent steps. As such, the apparatuses, systems, and methods may be considered portions of a two step conversion of biomass to hydrocarbon-like molecules and/or mixtures. In further processing steps the hydrocarbon-like molecules and/or mixtures are reacted to form liquid hydrocarbons, fuels, or biofuels, without limitation, or in other chemical products.

In general, biomass is fermented in a bioreactor to form a liquid feed for conversion hydrocarbons, hydrocarbon-like molecules, biofuels, or combinations thereof. The fermentation may comprise aerobic respiration, anaerobic fermentation, or combinations thereof. Examples of suitable fermentation systems or bioreactors, and methods may comprise those found in U.S. Pat. No. 5,962,307, U.S. Pat. No. 5,874,263, and U.S. Pat. No. 6,262,313, incorporated herein by reference, or others without limitation. Without limitation by theory, fermentation is the biological process of oxidizing organic compounds, such as but not limited to carbohydrates and proteins, to produce energy. Further, anaerobic fermentation produces a fermentation broth comprising acids, acid salts, acid esters, or combinations thereof.

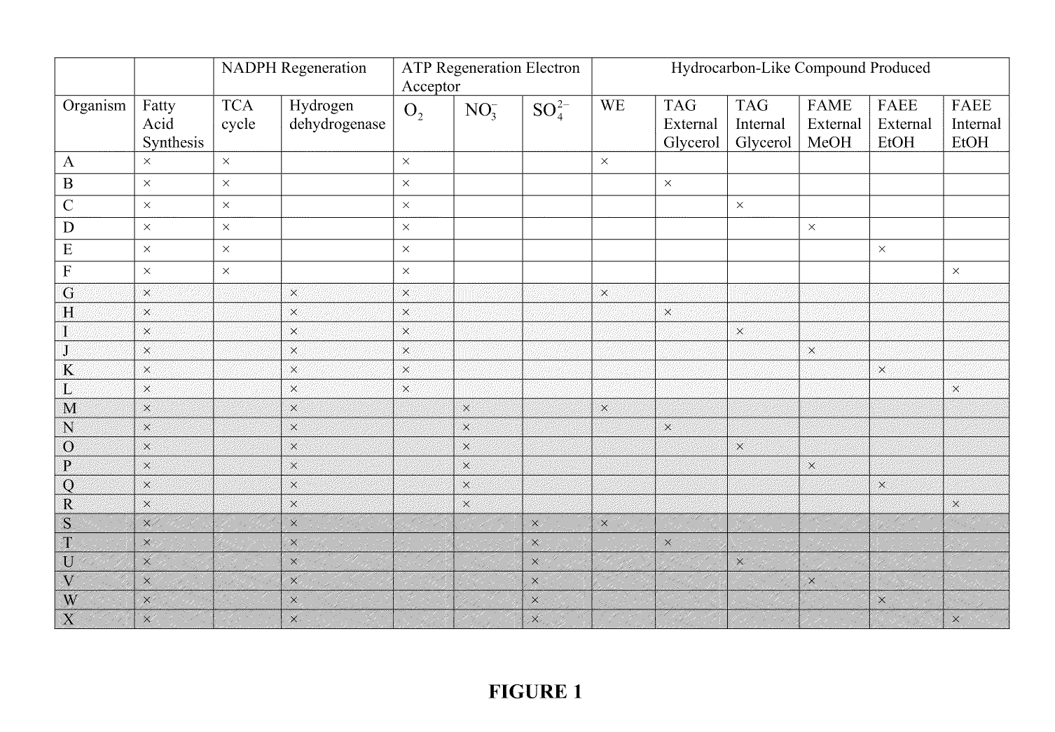

In general, the fermentation broth is removed and directed to microorganism mediated conversion. Without limitation by theory, the microorganism mediated conversion produces a product stream that comprises, waxy esters (WE), triacylglycerols or triacylglycerides (TAG), fatty acid methyl-esters (FAME), fatty acid ethyl-esters (FAEE), poly-hydroxyalkanoates (PHA), other hydrocarbons or lipids, and combinations thereof. As illustrated by the table in FIG. 1, any microorganism capable of producing these compounds by any conversion process from a fermenter broth may be suitable for use in the process. Further, the process may comprise a plurality of microorganisms producing different components of the product stream from the fermenter broth. The product stream is further processed into consumer products, including fuels, and other chemical commodities or products without limitation. The product stream processing may comprise refining, cracking, or blending to form liquid fuels from the hydrocarbons, hydrocarbon-like molecules, biofuels and combinations thereof. The product stream may be separated such that individual components are processed into separate chemical products such as but not limited to solvents, paints, additives, plastics, waxes, lubricants, and asphalt.

In embodiments, hydrocarbons comprise any molecule consisting primarily of carbon and hydrogen, including but not limited to aromatics, alkanes, alkenes, and alkynes having any molecular weight. Additionally, hydrocarbon-like molecules may comprise fats, lipids, waxes, proteins, alcohols, ketones, esters, acids, and combinations thereof, without limitation. Further, biofuels may comprise bioalcohols, biodiesels, vegetable oils, syngas, bioethers, and combinations thereof, without limitation. The hydrocarbons, hydrocarbon-like molecules, biofuels, and combinations thereof preferably maintain a liquid state at standard temperature and pressure. Alternatively, the hydrocarbons, hydrocarbon-like molecules, biofuels, and combinations thereof may at least partially be gaseous. In certain instances, hydrocarbons, hydrocarbon-like molecules, biofuels, or combinations thereof may comprise a mixture of liquids and gases.

Un-reacted and/or incompletely reacted compounds are recyclable throughout the apparatus and process. Alternatively, unreacted and/or incompletely reacted compounds may be directed to associated apparatus and processes. These processes may comprise biomass pretreatment, fermentation product separation, liquid fermentation product sterilization, microorganism lysing or combinations thereof. Further processes may include, without limitation, one or more process including biomass gasification, ammonia recovery, heat recovery, acid supplementation, hydrogen supplementation. In certain instances, at least a portion of the reaction by-products, co-products, contaminants, and/or waste are used for associated, supplemental, or ancillary processes. Without limitation by theory, one or more of these additional processes may include additional apparatuses integrated into the system used for the conversion of biomass to hydrocarbons and other chemical products.

### Fermentation

As described previously, the fermentation of biomass produces the acids, acid salts, or acid esters for conversion to the previously described compounds. In embodiments, the process of the fermentation of biomass comprises any fermentation process and apparatus understood by one skilled in the art capable of producing a broth of solubilized acids and/or acid salts, as used herein acids/salts and comprising carboxylic acids and carboxylate salts. In certain instances, anaerobic fermentation produces a suitable broth of solubilized acids/salts. The operation (e.g. residence time, loading rate, etc) and the size (volume) of the fermentor determine suitability of a fermentor broth for use in the present system, and the overall conversion efficiency of the fermentor and the system.

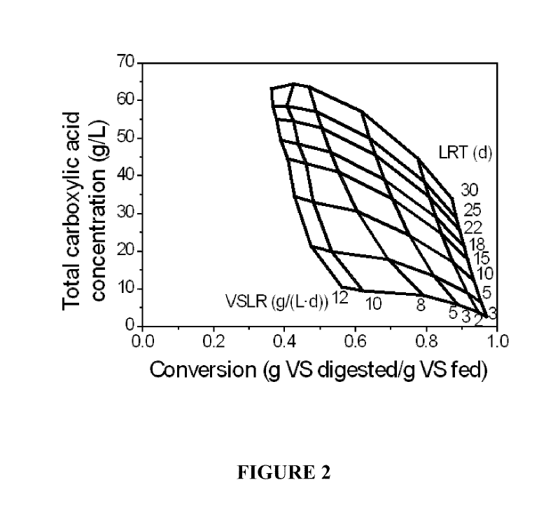

Referring now to FIG. 2 illustrating a hypothetical, total acid or acid salt concentration (TAC) versus conversion graph as determined by the residence time and solids loading rate. More specifically, the concentration of mixed acids and salts solubilized in the fermentation broth or as the fermentation broth is a product is a function of conversion, liquid residence time (LRT), and volatile solids loading rate (VSLR).

Without limitation by theory, the graph illustrates that at a volatile solids loading rate (VSLR) of ˜2.8 g/(L-day), a 30-day liquid residence time (LRT), with an 80% conversion rate will yield an acid/salt concentration of 43 g/L for a first fermentor. Alternatively, an acid/salt concentration of 8 g/L in the fermentor broth, with an 80% conversion of a VSLR of 8 g/(L-day), requires only an LRT of about 3 days in a second fermentor. Without limitation by theory, because the fermentor volume scales directly with VSLR, the second fermentor, including dilute acid/salt fermentor broth will require about one half to about one third the size of the first fermentor. The figures presented herein should not be interpreted as limiting, but rather as exemplary calculations related generally embodiments to follow herein.

In certain instances, methane may be present as a by-product of anaerobic fermentation. The formation of methane during fermenting lowers the acid/salt concentration and reduces the efficiency of the fermentation. Without limitation by theory, methanogenesis consumes the mixed acids and acid salts before they may be removed from the fermentation broth. Inhibitors such as but not limited to iodoform, and bromoform, are added with the biomass prior to fermentation in order limit methanogenesis.

Additionally, several types of buffers may be utilized to reduce methanogenesis and increase the acid production. Many buffering salts enter the fermentor with the biomass, which may contain a variety of cations. In non limiting examples, the cations may be sodium (Na), potassium (K), magnesium (Mg) and manganese (Mn), and additional quantities of such cations may be added as carbonates. Additional quantities of cations are needed for the physiological integrity of the microorganism and may be added as carbonates. Furthermore, the presence of certain cations during fermentation may increase the acids recovered from the fermentation. Further, the salts be precipitated or extracted from the dilute aqueous solution.

### Conversion

In general, the microorganism mediated conversion of fermentation products and broth comprises the biosynthesis of hydrocarbons, hydrocarbon-like molecules, or combination thereof to form a product stream. Without limitation by theory, microorganisms are capable of the uptake, conversion, and excretion and/or storage of hydrocarbon and hydrocarbon-like molecules. Additionally, microorganisms are capable of conversion, and excretion and/or storage by a plurality of metabolic pathways. Referring now to FIG. 1, the table illustrates exemplary, hypothetical organisms, capable of producing the molecules listed previously by different metabolic pathways.

The microorganisms that convert the fermentation broth may be heterotrophs, autotrophs, or mixotrophs. For the purpose of this following disclosure, heterotrophs are any microorganisms that use extracellular organic carbon sources for growth and biosynthesis. Autotrophs are any microorganisms that use extracellular inorganic carbon sources for growth and biosynthesis. Within the group of autotrophs there are photo-autotrophs that are any microorganisms that utilize light by photosynthesis, to convert inorganic carbon into organic molecules. Alternatively, some autotrophs are chemo-autotrophs (lithotrophs), or any microorganisms that use inorganic reactions for the reducing energy equivalents needed for organic synthesis and growth. Mixotrophic organisms are any microorganisms that are capable of alternating between heterotrophic and autotrophic growth and biosynthesis. In a nonlimiting example, a mixotroph may use extracellular organic energy, such as carbohydrates and sugars for growth and biosynthesis until those resources are depleted. With the depleted extracellular resource, the mixotroph may begin using inorganic reactions to provide the energy for growth and biosynthesis, and in certain instances, this microorganism may be considered a chemo-mixotroph. Further, a photo-mixotroph may be an organism that is capable of alternating between photo-autotrophic growth and lithotrophic growth. Without limitation by theory, many microorganisms that exist in anaerobic, extreme, or rapidly changing environments are chemo-autotrophs or chemo-mixotrophs. Aquatic or marine microorganisms are frequently heterotrophic, phototrophic, or photo-mixotrophic as their preferred environment and/or growth media is typically aerobic.

In non-limiting examples, many types of bacteria, yeast, algae, fungi, unicellular plants, and other microorganisms uptake acids and acid salts. The microorganisms convert acids and/or their salts to form hydrocarbons and hydrocarbon-like molecules. Anaerobic bacteria (e.g., Desulfovibrio desulfuricans) can assimilate the long-chain acids and/or their salts (e.g., carboxylates) that are self-secreted to produce intracellular lipids and esters. Similarly, other species (e.g., Marinobacter sp.) can uptake acetic acid and/or acetate to produce intracellular lipids. Additionally, many classes of bacteria accumulate polyhydroxyalkanoates (PHAs) that are derived from acid salts. Other bacteria (e.g., Acetobacter sp.) can synthesize wax esters (WE) from acids and/or acid salts. Certain yeast (e.g., Candida tropicalis) produce intracellular hydrocarbons when grown in certain media and increase production of hydrocarbons under anaerobic conditions. Algae (e.g., Nitzchia sp., Chlorella sp., and Chlamydomonas sp.) heterotrophically consume carboxylates, other acids, and their salts. Further, the Nitzchia sp. and Chlorella sp. can grow phototrophically as well and uptake CO₂ to form biomass in the presence of light. The lipids and esters produced from these exemplary species are easily converted and/or used in biodiesel production. These non-limiting example organisms produce intracellular and extracellular products that can be recovered by extraction from the cell mass. Further, non-limiting examples include heterotrophic organisms that secrete the desired hydrocarbons and/or hydrocarbon-like molecules as portions of the extracellular matrix. As above, many organisms are known to produce extracellular hydrocarbons from carboxylates (e.g., Desulfovibrio desulfuricans). Further, some of these microorganisms may be examples of hydrocarbon-producing mixotrophic organisms, as they utilize the acid, the acid salts, or CO₂ as a carbon source for molecular biosynthesis. Additionally, the microorganisms may use hydrogen gas (H₂) for reducing equivalents in biosynthesis of hydrocarbon and hydrocarbon-like molecules.

Without limitation by theory, the pathways for biosynthesis of hydrocarbons and/or hydrocarbon-like molecules utilize any combination of acids or acid salts from fermentation. The biosynthesis of hydrocarbons and/or hydrocarbon-like molecules includes waxy esters (WE), triacylglycerols or triacylglycerides (TAG), fatty acid methyl-esters (FAME), fatty acid ethyl-esters (FAEE), and poly-hydroxyalkanoates (PHA) without limitation. Further biosynthesis may comprise other hydrocarbons, lipids, esters, and combinations thereof that are converted from acids or acid salts by different biochemical processes.

In the following discussion and general equations related to the biosynthesis reactions that occur during microorganism mediated conversion, acetic acid is used to represent mixed acids. As may be understood by a skilled artisan, the reaction can involve other acids and acid salts such as propionic, butyric, valeric, caproic, heptanoic acids, their salts, and combinations thereof, without limitation. Additionally, salts may comprise calcium, sodium, potassium, magnesium, and manganese salts at physiological concentrations. Further, the carboxylate groups of the acetic acid will be expressed as non-ionized acids, although it maybe understood that the carboxylate groups exist in ionized form during the conversion process. Additionally, the process utilizes energy from the reduction of adenosine triphosphate (ATP) to adenosine diphosphate (ADP) or adenosine monophosphate (AMP) along with the formation of pyrophosphate (PPᵢ), though this reaction may not be explicitly shown or described. More specifically, the ATP derived water of hydrolysis may not be included in the reaction equations, but a skilled artisan will recognize the presence of this interaction.

The biosynthesis of fatty acids (e.g. FAEE, FAME) starts with the reaction of an acid (e.g. acetic acid) as a preliminary step to forming palmitic acid. The reaction of acetic acid with coenzyme A (H—S—CoA) is as shown in Equation 1.

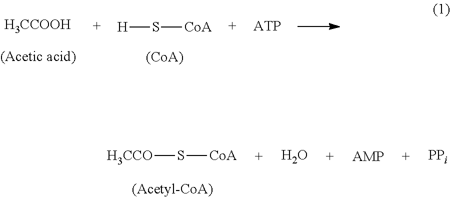

At a physiological pH, the acetic acid would be present as mixture of acetic acid and acetate, but for simplicity the unionized acetic acid is presented. Also, for simplicity the ATP water of hydrolysis is not included in the reactions and also for simplicity, although higher molecular weight acids are produced, only acetic acids is shown as an example, even though the reactions can involve higher acids like propionic, butyric, valeric, caproic, enanthic and caprylic acids. Without limitation by theory, Equation 1 may be considered energetically equivalent to Equation 2.

H₃CCOOH+H—S—CoA+ATP→H₃CCO—S—CoA+H₂O+ADP+Pᵢ (2)

Subsequently, the Acetyl-CoA molecules are reacted with carbon dioxide to form malonyl-CoA as in Equation 3.

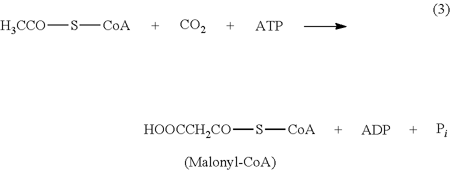

In a microorganism, nicotinamide adenine dinucleotide phosphate (NADPH) reduces one acetyl-CoA and seven malonyl-CoA molecules react to form a lipid (e.g. palmitic acid).

H₃CCO—S—CoA+7HOOCCH₂CO—S—CoA+14NADPH+14H+→H₃C(CH₂)₁₄COOH+7CO₂+8HS—CoA+14NADP⁺+6H2O (4)

To maintain the biosynthesis reactions, the microorganism's enzyme CoA undergoes aerobic regeneration through the tricarboxylic acid cycle (TCA). In general, the acetyl-CoA reacts as in Equation 5.

H₃CCO—S—CoA+3H2O+3NADP++FAD+GDP+P_{i→}2CO₂+H—S—CoA+3NADPH+H⁺+FADH₂+GTP (5)

Without limitation by theory, this is energetically equivalent to Equation 6.

H₃CCO—S—CoA+3H₂O+4NADP⁺+ADP+Pᵢ→2CO₂+H—S—CoA+4NADPH+H⁺+ATP (6)

Further, by electron transport and oxidative phosphorylation, the presence of NADPH+H₊creates regenerates ATP by reaction with oxygen as in Equation 7.

NADH+H⁺+3ADP+3Pᵢ+½O₂→NAD⁺+3ATP+H₂O (7)

Equation 7 may be energetically equivalent to Equation 8.

NADPH+H⁺+3ADP+3Pᵢ+½O₂→NADP⁺+3ATP+H₂O (8)

As such, using the above equations to determine the overall balanced equation for aerobic metabolism of acetic acid would therefore be reduced as shown in Equation 9.

12.75(Eq. 2)+7(Eq. 3)+(Eq. 4)+4.75(Eq. 6)+5(Eq. 8) (9)

Without limitation by theory, the higher heat of combustion for acetic acid is 871.69 kJ/mol and the higher heat of combustion for palmitic acid is 11,094 kJ/mol. Therefore the efficiency for such a conversion, is:

$$η = \frac{11 , 094 kJ \text{/} mol}{12.75 ( 871.69 kJ \text{/} mol )} = 0.998 \qquad ( 10 )$$

As may be understood by a skilled artisan, Equation 5 illustrates that oxygen is the electron acceptor. However, any electron acceptor utilized by a microorganism may be used. For example, nitrate (NO₃⁻) can serve as an electron acceptor to form nitrite (NO₂⁻), nitrous oxide (N₂O), di-nitrogen (N₂), ammonium (NH₄⁺) or other nitrogenous molecules; alternatively, sulfate (SO₄⁻²) may be the electron acceptor to form hydrogen sulfide (H₂S), without limitation.

Further, the anaerobic regeneration of NADP' may use hydrogen dehydrogenase as in Equation 11.

H₂+NADP⁺→NADPH+H⁺ (11)

The regeneration of ATP may include at least some oxidation as shown in the balanced Equation 12.

8(Eq. 1)+7(Eq. 3)+(Eq. 4)+5(Eq. 8)+19(Eq. 6) (12)

The higher heat of combustion for hydrogen is 285.84 kJ/mol, and the higher heat of combustion for acetic acid is 871.69 kJ/mol and for palmitic acid is 11,094 kJ/mol, as above. Therefore the efficiency of the reaction, taking into account the oxidation of the ATP is shown in Equation 13.

$$η = \frac{11 , 094 kJ \text{/} mol}{8 ( 871.69 kJ \text{/} mol ) + 19 ( 285.84 )} = 0.894 \qquad ( 13 )$$

Further, the microorganisms may synthesize lipids and esters from free fatty acids. As understood by one skilled in the art, free fatty acids may damage cell membranes, organelles, or DNA when left intracellularly. Synthesizing lipids and esters, storing the lipids and esters for reserve energy, and/or using lipids and esters for construction of cellular membranes prevents damage and disruption of the membranes. The lipids may comprise fatty acids, waxes, sterols, fats (glycerides), or other unsaturated hydrophobic molecules, without limitation. In some instances, the microorganisms may convert free fatty acids to esters metabolically. For example, two fatty acids can react together to form an ester by converting one of the acids to an alcohol. More specifically, fatty acids or lipids may be reacted to form a triacylglycerol, a fatty acid methyl-ester (FAME), or a fatty acid ethyl-ester (FAEE). The ATP and NADPH utilized by the microorganism for energy may be regenerated using the methods described previously herein.

In the synthesis of an ester, the enzyme acyl-CoA synthetase found in many microorganisms may activate two fatty acids to form fatty acyl-CoA as shown in Equation 14.

H₃C(CH₂)₁₄COOH+H—S—CoA+ATP→H₃C(CH₂)₁₄CO—S—CoA+H₂O+AMP+PPᵢ (14)

The enzyme acyl-CoA reductase found in many microorganisms catalyzes the reaction forms an aldehyde from one of the activated fatty acids as shown in Equation -b 15.

H₃C(CH₂)₁₄CO—S—CoA+NADPH+H⁺→H₃C(CH₂)₁₄COH+H—S—CoA+NADP⁺ (15)

The enzyme fatty aldehyde reductase found in many microorganisms, reduces this aldehyde to form an alcohol as in Equation 16.

H₃C(CH₂)₁₄COH+NADPH+H⁺→H₃C(CH₂)₁₄CH₂₋OH+NADP⁺ (16)

Further, the enzyme wax ester synthase, found in many microorganisms, using the alcohol from Equation 16 reacts with the product of Equation 14 to form a wax ester as shown by Equation 17.

H₃C(CH₂)₁₄CO—S—CoA+H₃C(CH₂)₁₄CH₂OH→H₃C(CH₂)₁₄COOCH₂(CH₂)₁₄CH₃+HS—CoA (17)

Alternatively, an organism may complete the pathway for the biosynthesis of triacylglycerol or triacylglycerides (TAG). The reaction uses the enzyme glycerol kinase found in many microorganisms with free glycerol produced by the cell, and results in the phosphorylation of glycerol to form glycerol phosphate:

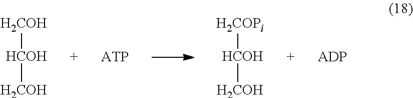

The enzyme glycerolphosphate acyltransferase found in many microorganisms, takes fatty acyl-CoA (Eq. 14) and reacts to regenerate acyl-CoA, and a glyceride phosphate:

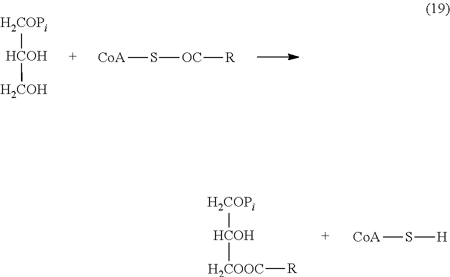

wherein —R represents the hydrocarbon tail of the fatty acid.

The enzyme glycerolphosphate acyltransferase, and fatty acyl-CoA (Eq. 14) reacts with the glyceride phosphate to add a second fatty acid hydrocarbon to the glycerol, as in Equation 19:

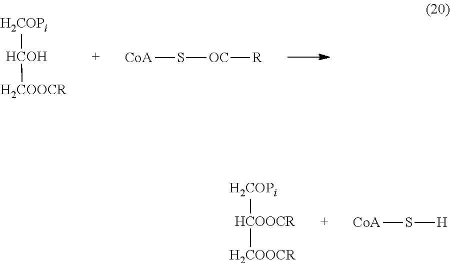

After transferring the fatty acids tails to the glycerol, the enzyme phosphatidate phosphatase found in many microorganisms, removes the inorganic phosphate from the glycerol:

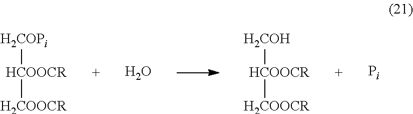

Finally, using the enzyme diacylglycerol acyltransferase, fatty acyl-CoA (Eq. 14) also reacts:

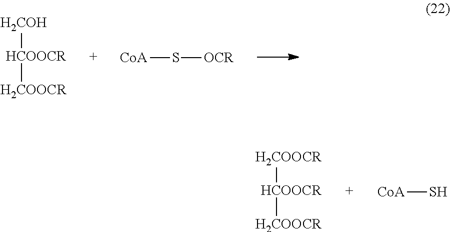

In further instances, the enzyme fatty acid ethyl ester synthase, in the absence of CoA reacts free fatty acids, for example palmitic acid without limitation, and ethanol to form FAEE and/or FAME. In certain instances, the ethanol may be added to the process or it may be synthesized by the organism. In FAEE synthesis:

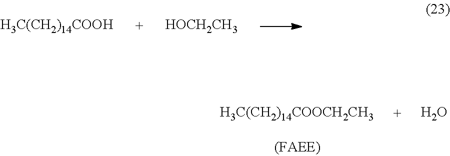

In many microorganisms, the enzyme aldehyde dehydrogenase uses Acetyl-CoA (Eq. 1) for the synthesis of an acetaldehyde and the regeneration of enzyme CoA:

H₃CCO—S—CoA+NADH+H⁺→H₃CCOH+H—S—CoA+NAD⁺ (24)

Sequentially, the enzyme alcohol dehydrogenase found in many microorganisms, may convert the acetaldehyde to form ethanol:

H₃CCOH+NADH+H⁺→H₃CCH₂OH+NAD⁺ (25)

The ethanol produced in this reaction may be used for the continued synthesis of the FAEE.

Alternatively, fatty acids (e.g. palmitic acid) may be reacted with methanol, for FAME synthesis:

H₃C(CH₂)₁₄COOH+HOCH₃→H₃C(CH₂)₁₄COOCH₃+H₂O (26)

As previously discussed, all, any, some, or none of the lipid and/or ester products may be stored internally. Further, the lipids and/or esters may be excreted or make up part of the extra-cellular matrix of the microorganisms. Further, other hydrocarbon or hydrocarbon-like molecules may be formed by alternative biosynthetic pathways though they are not discussed herein. One of skill in the art will recognize that other lipids, fatty acids, and esters may be formed from the conversion of acids and acid salts by microorganisms.

### Processing

Processing comprises any steps for refining the lipids, esters, and other hydrocarbons or hydrocarbon-like molecules to an end product, such as liquid fuel. For example in liquid fuels production, processing comprises refining, cracking, alkylating, polymerizing, and separating without limitation. In embodiments of liquid fuels, processing comprises refining the hydrocarbon and hydrocarbon-like molecules to form gasoline, diesel, kerosene, jet fuel, solvents, lubricants, olefins, alkylolefins, commodity chemicals, and combinations thereof. The processes of refining to liquid fuels comprises forming six-carbon to twelve-carbon chains; alternatively, forming eight-carbon to twenty-one carbon chains; and alternatively, six-carbon to sixteen-carbon chains. Refining the hydrocarbon and hydrocarbon-like molecules may further comprise forming any hydrocarbon liquid having a carbon chain between about one carbon and about thirty carbons.

Processing may comprise catalytically cracking the hydrocarbon and hydrocarbon-like molecules to form light hydrocarbons or short chain hydrocarbons. Alternatively, processing may comprise polymerizing the hydrocarbon and hydrocarbon-like molecules to form waxes, lubricants, gels, and plastics. Further processing may comprise transesterification, hydrogenation, decarboxylation, isomerization, cleaving, and crosslinking in nonlimiting examples. In each of the possible processing steps described herein, the hydrocarbon and hydrocarbon-like are separated from any remaining cellular components, membranes, enzymes, proteins, and the like prior to delivering the final or consumer product.

### Heterotrophic Methods

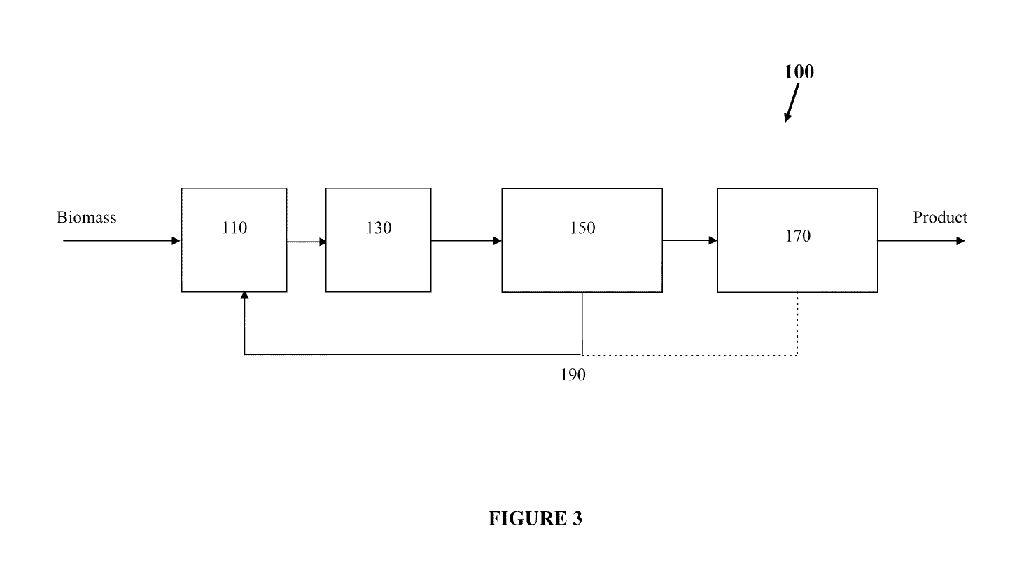

Referring now to FIG. 3, illustrating a general flow diagram for a process 100 for converting acids, acid salts, and combinations thereof to chemical products or hydrocarbon products. The process 100 includes hydrocarbons, hydrocarbon-like molecules, or combinations thereof produced by heterotrophic organisms. The process 100 comprises introducing biomass to a fermenter 110, separating liquid fermenter products 130, converting the liquid fermenter products 150 for example to form conversion products, and processing the conversion products 170, for example into chemical products or hydrocarbon products. Some products are recycled 190 back through the process 100 by re-introduction to the fermentation step 110 from the conversion 150 and processing 170 steps.

In embodiments FIG. 3 is a process flow diagram for the integration of anaerobic fermentation 110 and heterotrophic conversion 150 into a process 100. Fermentation 110 generally comprises a variety of anaerobic bacteria converting biomass into mixed acids or acid salts, herein acids/salts. Without limitation by theory, suitable biomass comprises any biological material that ferments to form acids and acid salts in solution. The resulting acid/salt solution is separated 130 and fed to heterotrophic conversion 150. During the conversion 150, at least one heterotrophic organism converts the acids/salts solution into hydrocarbons or hydrocarbon-like conversion products. The conversion products are processed 170 into biofuels, biochemical products, or other chemical commodities, without limitation. Water, intact and lysed cells, macromolecules, byproducts, and unreacted acids/salts from the heterotrophic conversion 150 and processing 170 may be redirected for recycling 190 to recover acids/salts, by-products, biofuels, biochemical, or other components. In certain instances, the heterotrophic conversion step 150 returns organic materials to the recycling step 190 for fermentation 110 and the processing step 170 returns water and dilute solutions. Alternatively, only one process chosen from the conversion step 150 and the processing step 170 feeds the recycling step 190.

In the following discussion and illustrations of various embodiments of the general process discussed hereinabove, similar processes and pathways are noted by similar reference numerals. For example, the step of fermentation 110 may be indicated as 210, 310, 410, etc in the subsequent figures and discussion. Additionally, the step of conversion 150 may be indicated as 250, 350, 450, etc. While the general steps may be related, the specific properties, reactions, and products of the general steps may differ, and therefore should not be limited to any particular embodiment described in a preceding discussion, or shown in a preceding illustration.

### First Integrated Process

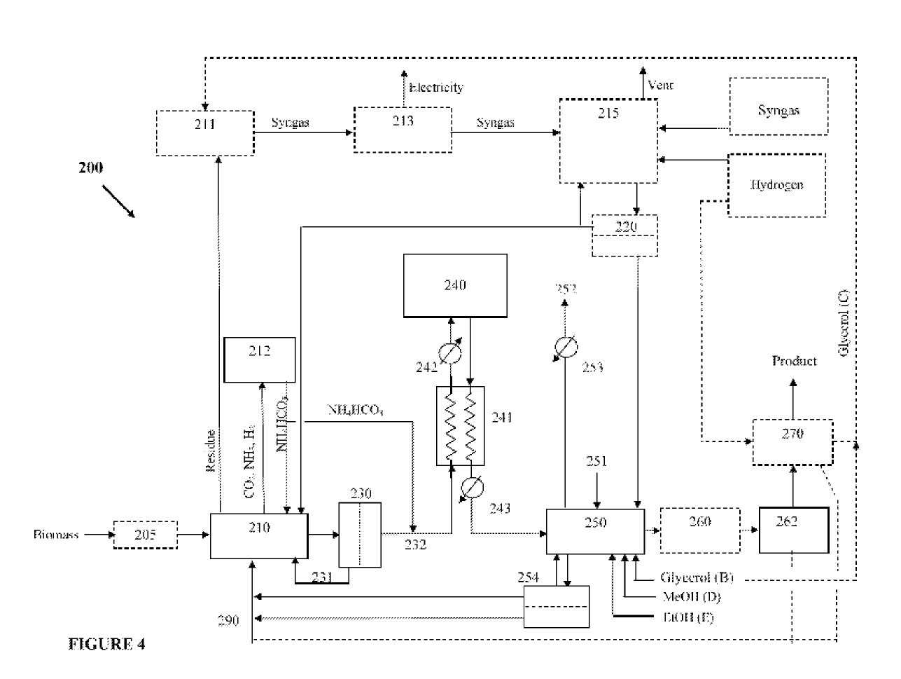

Referring now to FIG. 4 illustrating an embodiment of the process generally shown in FIG. 3, the process 200 for converting biomass to hydrocarbon products, the process generally comprises the steps of fermentation 210, withdrawing 230 an acids/salts solution, converting 250 the acids/salts solution to conversion products, processing the conversion products 270 to hydrocarbon products, and optionally recycling 290 a portion of the products.

In embodiments, the process 200 is configured to integrate a digestion or fermentation for production of mixed carboxylic acids/salts and a fermentor with at least a portion of organisms shown in FIG. 1 for biosynthesis of hydrocarbons and/or hydrocarbon-like molecules. More specifically, the process 200 is for the integration of digestion or fermentation with heterotrophic organisms, such as organisms A through F. In embodiments, the organisms A through F of FIG. 1, comprise heterotrophic organisms that convert fermentation products, including mixed acids and salts, by the TCA cycle for regeneration of NADPH. Further, as shown in FIG. 1 the organisms A through F are aerobic, in that they may utilize oxygen as an electron acceptor, or oxidant.

Referring again to FIG. 4, biomass is introduced to a fermentor for the process of fermentation 210. In embodiments, biomass such as the nonlimiting examples, municipal solids waste, farm waste, lignocellulosic/starchy crops, or combinations thereof, are digested during fermentation 210. Fermentation 210 conditions favor the production of mixed acids and acid salts in the fermentation broth.

Optionally, the biomass is pretreated 205 prior to fermentation 210. In these embodiments, the biomass has a high lignin content that is insoluble, indigestible, and/or interferes with the mixed acid fermentation. Nonlimiting examples of potential pretreatment processes include sulfuric acid pretreatment, hot water pretreatment, steam pretreatment or autoclaving, ammonia pretreatment, ammonia-fiber expansion (AFEX), and lime pretreatment. Additional pretreatment processes may be found for example in U.S. Pat. No. 5,865,898, U.S. Pat. No. 5,693,296, or U.S. Pat. No. 6,262,313, without limitation. After pretreatment 205, the pretreated biomass is subjected to mixed acid fermentation 210.

After fermentation 210 the fermentation broth comprising the mixed acids/salts is separated 230. In embodiments, the fermentation broth comprises non-sterile suspension or colloid including biomass debris, suspended solids, cellular debris, microorganisms, acids/salts and other fermentation products. In embodiments, separating 230 the fermentation broth further comprises separating the solids from the liquids. The solids including biomass debris, macroscopic suspended solids and particles are screened, filtered, settled, centrifuged, or decanted from the unsterilized liquids including microorganisms, microscopic suspended solids, cellular debris and the acids/salts. The separated solids are returned 231 for further digestion and fermentation 210 to acids/salts. The non-sterile liquids comprising acids/salts are removed 232 from separation 230 to conversion 250.

In embodiments the non-sterile liquids, comprising the acids/salts are sterilized 240 prior to conversion 250. The sterilization 240 of the fermentation broth liquids comprises, without limitation, thermal, pressure, autoclaving, UV, and combinations thereof, to form a sterilized acids/salts broth. Further, the fermentation broth may be sterilized 240 in a batch process. A batch process may allow a longer residence time at the sterilization temperature. Without limitation by theory, increased residence time at the sterilization temperature lyses and kills the fermentation microorganisms in the broth and degrades enzymes and other proteins that may negatively impact the conversion of the acids/salts in conversion process 250. Alternatively, without limitation, sterilization 240 comprises a continuous flow process, such as without limitation, through a plug-flow reactor. Without limitation by theory, continuous flow sterilization reduces deposition or settling of suspended solids in the sterilization apparatus.

In embodiments, the sterilization 240 comprises elevating the temperature of the fermentation broth to above about 100° C.; alternatively, to above 110° C.; and in certain instances over about 140° C. The sterilization 240 further comprises heating the fermentation broth with steam 242. In certain embodiments, the fermentation broth is sterilized for at least about 3 minutes; alternatively, for at least about 5 minutes; and alternatively, for at least about 10 minutes. Alternatively, the fermentation broth is sterilized in by continuously filling a sterilization reactor, sterilizing the fermentation broth, and draining the sterilized broth comprising the acids/salts.

In order to conserve, reuse, or recycle thermal energy within process 200, heat exchange 241 between the non-sterile fermentation broth and the sterilized broth may be implemented. Without limitation by theory, heat exchange 241 warms the unsterilized broth prior to introduction of steam 242. Warming the unsterilized broth by heat exchange 241 reduces the volume, temperature, and pressure of the steam introduction 242. Additionally, heat exchange 241 at least partially cools the sterilized acids/salts prior to conversion, for example the biological conversion 250. In embodiments, the sterilized broth is further cooled 243 prior to conversion by heat exchange with water. As above, to conserve, reuse, or recycle thermal energy within process 200, the water from cooling 243 having been warmed by thermal energy from the sterilized broth may be used for other purposes, in non-limiting examples for steam introduction 242 and sterilization 240. In embodiments, the cooled, sterilized broth is directed to conversion 250.

In the present process 200, the conversion 250 is a heterotrophic conversion. The conversion 250 forms hydrocarbon and/or hydrocarbon-like products such as WE, TAG, FAME, FAEE, PHAs, other hydrocarbons, and combinations thereof, as described in detail hereinabove. In further embodiments, the hydrocarbon-like products may comprise hydrocarbon alcohols (e.g. hexanol), ketones, and/or aldehydes, without limitation. In embodiments, the hydrocarbons and/or hydrocarbon-like products may be externalized as extracellular matrix molecules or as extracellular secretions. In alternate embodiments, the hydrocarbons and/or hydrocarbon-like products are produced intracellularly.

Referring to FIG. 1 in relation to process 200 of FIG. 4, the heterotrophic organisms include the metabolic configurations of A-F for the conversion of the acids/salts. Organisms A-F convert the acids/salts into hydrocarbon and/or hydrocarbon-like products by aerobic biosynthetic paths. In embodiments, air or oxygen (O₂) is introduced 251 during conversion 250 to act as the electron acceptor at the end of the TCA cycle as an oxidant. In certain instances, conversion 250 includes introducing additional reactants for conversion 250. Non-limiting examples of additional reactants include glycerol, which can be processed by Organism B, methanol, which can be processed by Organism D, or ethanol, which can be processed by Organism E. In still other embodiments, conversion 250 comprises venting or releasing waste gases 252 such as CO, CO₂, or N₂. However, measures may be taken to avoid losing the volatile reactants in the conversion. In certain instances, cooling and condensing 253 the gases being vented is suitable to recover volatile reactants. Conversion may further require cooling or heating the conversion reaction to improve the conversion efficiency, conversion rate, reactant recovery, or optimize conditions for the microorganisms.

The conversion process 250 may include selectively separating microorganisms 254 for recycling within the conversion process 250. In embodiments where microorganisms produce hydrocarbons and/or hydrocarbon-like molecules that are extracellular matrix molecules or extracellular excretions lysis 260 of the microorganisms. As understood by a skilled artisan, there are many ways to recover the hydrocarbons and/or hydrocarbon-like molecules, and in instances the hydrocarbon or hydrocarbon-like molecules tend to be immiscible and therefore float to the surface of aqueous solutions. In embodiments, the extracellular hydrocarbons and/or hydrocarbon-like molecules are decanted or skimmed and directed to processing 270, without limitation.

The remaining suspension comprising the microorganisms, unconverted acids/salts, and conversion media liquid are directed to separation 254. Separation 254 may comprise filtering, settling, washing, centrifuging, or other methods to remove the microorganisms from the liquid without limitation. The liquid comprises a suspension comprising unconverted acids/salts, waste products, dead microorganisms, and other suspended solids, without limitation. In embodiments, the liquid is recycled 290 for fermentation 210. Additionally, solids such as the unconverted acids/salts, waste products, dead microorganisms, and other suspended solids are also recycled 290 to fermentation 210. The liquids may be recycled 290 to fermentation 210 concurrently or separately from the unconverted acids/salts, waste products, dead microorganisms, and other suspended solids. In embodiments, the microorganisms may be returned to the conversion 250 of further sterilized acids/salts.

Alternatively, in embodiments where the microorganisms produce intracellular hydrocarbon and/or hydrocarbon-like molecules, the microorganisms are subjected to lysing 260. Lysing 260 further comprises concentrating the microorganism cell mass for example by centrifugation or flocculation, without limitation. Lysing 260 may comprise any process suitable for rupturing a cell membrane and solubilizing the intracellular matrix known to a skilled artisan. Nonlimiting examples of lysing 260 including centrifuging, osmotic shocking, supercritical fluid extraction, solvent extraction, cold pressing, shearing, homogenizing, blending, milling, sonication, or other techniques.

In embodiments, lysing 260 the microorganisms comprises recovering 262 the hydrocarbons and/or hydrocarbon-like molecules from the other cellular components, comprising proteins, enzymes, membranes, nucleic acids and liquids from the lysed microorganisms. As previously described, there are many ways to recover the hydrocarbons and/or hydrocarbon-like molecules, and in instances the hydrocarbon or hydrocarbon-like molecules tend to be immiscible and therefore float to the surface of aqueous solutions. In embodiments, the extracellular hydrocarbons and/or hydrocarbon-like molecules are decanted or skimmed and directed to processing 270, without limitation. Alternatively, the hydrocarbon and hydrocarbon-like molecules may be aggregated with other cellular components that are immiscible or hydrophobic. As such, to separate the hydrocarbon and/or hydrocarbon-like molecules, any process known to a person of skill in the art may be used, including membrane separation, filtering, and centrifuging. The other cellular components, comprising proteins, enzymes, membranes, and liquids are recycled 290 to fermentation 210. Intracellular liquids may be recycled 290 to fermentation 210 concurrently or separately from the other cellular components.

In embodiments, whether from extracellular production or cell lysing and recovery, the hydrocarbon and/or hydrocarbon-like molecules are directed to processing 270. Without limitation, processing 270 may chemically convert the hydrocarbon and/or hydrocarbon-like molecules into chemicals, solvents, or hydrocarbon fuels that are compatible with the present fuel infrastructure. In the non-limiting examples the WE, TAG, FAME, FAEE, and PHAs previously discussed herein, processing may comprise transesterification (e.g. TAG), hydrogenation, decarboxylation, isomerization, cleaving, cross-linking, and other hydrocarbon reactions, such as refining, cracking, alkylating, polymerizing, and separating. The processing 270 of the hydrocarbon and/or hydrocarbon-like molecules may further comprise incorporation of hydrogen (H₂).

The process 200 may integrate other methods and processes. Without limitation by theory, integration of other steps, feeds, and processes into the process 200 reduces capital cost, improves raw material usage, and improves operational efficiency and flexibility. Nonlimiting process examples include gasification 211 to produce syngas, ammonia recovery 212, and electricity generation 213. Further, the process 200 may directly or indirectly supplement the production of electricity 213 from the formation of syngas. In certain embodiments, the undigested residue from fermentation 210 may be gasified 211, and the gasified residue may be used for syngas or syngas production. The syngas production may be used in electricity generation 213, as thermal energy derived from cooling the gasification products, comprising syngas, carbon monoxide, carbon dioxide, hydrogen, other organic gases, and combinations thereof, may be used to generate electricity (e.g. via a co-generation process). All or a portion of the products of gasification 211 may be used in electricity generation 213 and/or may be passed to other downstream process such as a chemoautotrophic process or hydrocarbon recovery, without limitation. Additionally, excess supplemental glycerols, from conversion feeds (e.g. by Organism B,C; FIG. 1) may be used for gasification.

Alternatively or additionally, the syngas may be used for other microorganism mediated processes 215. In certain embodiments, the syngas may be converted to acids/salts by a chemoautotrophic microorganism in process 215. The chemoautotrophic microorganism may comprise pure, mixed, natural, or genetically modified cultures. The acids/salts derived from chemoautotrophic process 215 may be recovered in separator 220 and used to supplement those from fermentation 210 for conversion 250. Chemoautotrophic process 215 may additionally supply feedstocks for fermentation 210 in the form of waste products and excess and dead microorganisms from separator 220, or recycle same to chemoautotrophic process 215.

In instances, supplemental sources of synthesis gas and hydrogen, such as reformed natural gas or electrolyzed water, may feed the chemoautotrophic process 215. And in certain circumstances, the entire process 200 may run on supplemental sources of synthesis gas or hydrogen. In these embodiments the process 200 is an example of gas-to-liquids conversion.

In additional embodiments, the gases produced during fermentation 210 comprise a mixture of ammonia (NH₃), carbon dioxide (CO₂), and hydrogen (H₂). Recovery and redirection of fermentation gases 212 captures and recycles these and other gases through process 200. For example, NH₃ is recovered during a packed bed reaction with CO₂. The recovered NH₃ is converted to ammonium bicarbonate (NH₄HCO₃) for recycle to fermentation 210 (e.g. for pH control) and/or incorporation in the acids/salts stream for conversion 250.

In regards to glycerol and TAG, supplemental glycerol may be used during conversion 250 for certain organisms (e.g. Organism B, C; FIG. 1). Additionally, glycerol may be synthesized during conversion 250 (e.g. Organism C; FIG. 1). Excess glycerol resulting from supplemental feeds and conversion may be recovered during processing 270, supplemental and/or recycled glycerol may be fed to gasification 211 for conversion to syngas, directed to the chemoautotrophic process 215, returned for conversion 250 for heterotrophic metabolizing to produce more TAG, or combinations thereof.

### Second Integrated Process

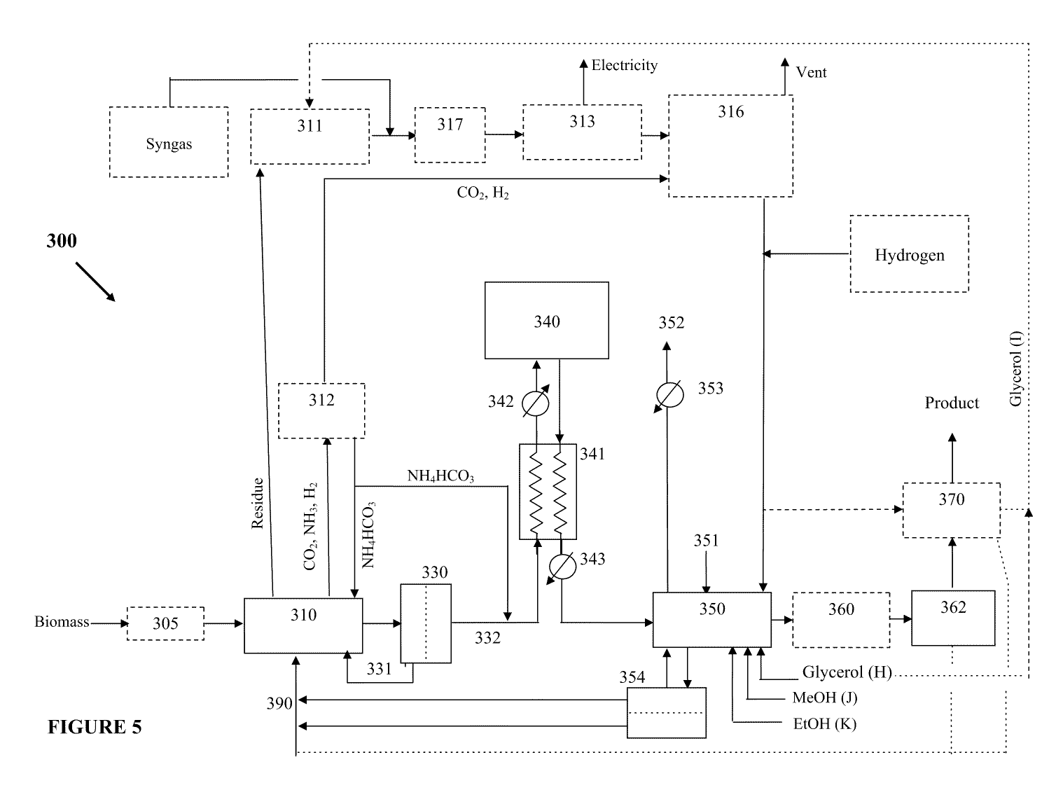

Referring now to FIG. 5 illustrating a second embodiment of the process generally shown in FIG. 3, the process 300 for converting biomass to hydrocarbon products, the process generally comprises the steps of fermentation 310, withdrawing an acids/salts solution 330, converting the acids/salts solution through conversion 350 into products, processing the conversion products to hydrocarbon products using conversion 370, and recycling 390 a portion of the products and by-products.

In embodiments, the process 300 is configured to integrate a fermentor with at least a portion of organisms shown in FIG. 1 for biosynthesis of hydrocarbons and/or hydrocarbon-like molecules. More specifically, the process 300 is for the integration of heterotrophic organisms, such as organisms G through L. In embodiments, the organisms G through L in FIG. 1 comprise heterotrophic organisms that convert fermentation products, including mixed acids and salts, using the enzyme hydrogen dehydrogenase for regeneration of NADPH. Further, as shown in FIG. 1 the organisms G through L are aerobic, in that they utilize oxygen as an electron acceptor.

The process 300 includes similar steps as process 200 illustrated in FIG. 4 and discussed previously. More specifically, process 300 includes biomass, which is introduced to a fermentor for the process of fermentation 310. In embodiments, biomass such as the nonlimiting examples, municipal solids waste, farm waste, lignocellulosic/starchy crops, or combinations thereof, are digested during fermentation 310. Optionally, the biomass is pretreated 305, by any method, prior to fermentation 310 to reduce or degrade lignin in high lignin content biomass. Fermentation 310 conditions favor the production of mixed acids and acid salts in the fermentation broth.

In embodiments, the fermentation broth comprising the mixed acids/salts is separated 330, and solids 331 are returned to the fermentation 310. The remaining liquid fermentation broth comprises an non-sterile suspension including microorganisms, microscopic suspended solids, cellular debris and the acids/salts. The non-sterile liquids comprising acids/salts are removed 332 from separation 330 for sterilization 340.

In embodiments of the process 300, the sterilization 340 comprises elevating the temperature of the fermentation broth to above about 100° C.; alternatively, to above 110° C.; and in certain instances over about 140° C., with steam 342 for at least about 3 minutes; alternatively, for at least about 5 minutes; and alternatively, for at least about 10 minutes. Additionally, in order to conserve, reuse, or recycle thermal energy within process 300, heat exchange 341 between the non-sterile fermentation broth 332, the sterilized acids/salts from sterilization 340, water from cooling 343 and steam 342 may be implemented as previously described. In embodiments, the cooled, sterilized acids/salts are directed to conversion 350.

In the present process 300, the conversion 350 is a heterotrophic conversion. Referring to FIG. 1 in relation to the process 300 shown in FIG. 5, the heterotrophic organisms include the metabolic configurations of G through L for the conversion of the acids/salts. Organisms G through L convert the acids/salts into hydrocarbon and/or hydrocarbon-like products by aerobic biosynthetic paths, using introduced air or oxygen (O₂) 351 as the electron acceptor or oxidant after hydrogen dehydrogenase NADPH regeneration and ATP regeneration.

In further embodiments, conversion 350 comprises introducing additional reactants for conversion 350. Non-limiting examples of additional reactants include hydrogen, glycerol (which may be processed by organism H), methanol (which may be processed organism J), or ethanol (which may be processed by organism K). In still other embodiments, conversion 350 comprises venting or releasing waste gases 352 such as CO or CO₂. However, to avoid losing the volatile reactants, cooling and condensing the vented gases 353 may be suitable. Additionally, conversion 350 may further require cooling or heating the conversion reaction to improve the conversion efficiency, conversion rate, reactant recovery, or optimize conditions for the microorganisms.

The conversion 350 forms hydrocarbon and/or hydrocarbon-like products such as WE, TAG, FAME, FAEE, PHAs, other hydrocarbons, and combinations thereof, as described in detail hereinabove. In further embodiments, the hydrocarbon-like products may comprise hydrocarbon alcohols (e.g. hexanol), ketones, or aldehydes, without limitation. In embodiments, the hydrocarbons and/or hydrocarbon-like products may be externalized as extracellular matrix molecules or as extracellular secretions. In alternate embodiments, the hydrocarbons and/or hydrocarbon-like products are intracellular molecules.

The conversion process 350 may include selectively separating microorganisms 354 for recycling within the conversion process 350. In embodiments, microorganisms produce hydrocarbons and/or hydrocarbon-like molecules and do not require lysing 360 the microorganisms. As understood by a skilled artisan, the hydrocarbon or hydrocarbon-like molecules may be immiscible, floating to the surface of aqueous solutions, such that they may be decanted or skimmed for processing 370, without limitation.

The remaining suspension comprising the microorganisms, unconverted acids/salts, and conversion media liquid are directed to separation 354. Separation 354 may comprise filtering, settling, washing, centrifuging, or other methods to remove or separate the microorganisms from the liquid. In embodiments, the microorganisms may be returned for the conversion 350 of further sterilized acids/salts. In further embodiments, the liquid may be recycled 390 for fermentation 310 to salts/acids or returned to conversion 350. Additionally, solids such as the unconverted acids/salts, waste products, dead microorganisms, and other solids are recycled 390 for fermentation 310 to acids/salts.

Alternatively, in embodiments where the microorganisms produce intracellular hydrocarbon and/or hydrocarbon-like molecules, the microorganisms are subjected to lysing 360. Lysing 360 further comprises concentrating the microorganism cell mass, rupturing the cell membranes and solubilizing the intracellular matrix by any processes known to a skilled artisan and discussed previously. In embodiments, lysing 360 the microorganisms comprises recovering 362 the hydrocarbons and/or hydrocarbon-like molecules from the other cellular components. In instances the hydrocarbon or hydrocarbon-like molecules may be immiscible, float to the surface of aqueous solutions for skimming or decanting for processing 370. The other cellular components are optionally recycled 390 for fermentation 310.

In embodiments, the hydrocarbon and/or hydrocarbon-like molecules are directed to processing 370. Without limitation, processing 370 may comprise the synthesis of chemicals, solvents, or hydrocarbon fuels that are compatible with the present fuel infrastructure. In the nonlimiting examples the WE, TAG, FAME, FAEE, and PHAs previously discussed herein, processing may comprise transesterification (e.g. TAG), hydrogenation, decarboxylation, isomerization, cleaving, crosslinking, and other hydrocarbon reactions, such as refining, cracking, alkylating, polymerizing, and separating. The processing 370 of the hydrocarbon and/or hydrocarbon-like molecules may further comprise incorporation of hydrogen (H₂) from any source.

The process 300 may integrate other methods and processes. Without limitation by theory, integration of other steps, feeds, and processes into the process 300 reduces capital cost, improves raw material or feedstock usage, and improves operational efficiency and flexibility. Nonlimiting process examples include gasification 311, ammonia recovery 312, and hydrogen purification 316. Further, the process 300 may directly or indirectly supplement the production of electricity 313 from the formation of syngas, hydrogen, and the recovery of thermal energy therefrom.

In embodiments, undigested residues from the fermentation 310 are gasified 311 as described previously. The gasified residue products comprise mixtures of H₂O, CO, CO₂, and H₂. Without limitation by theory, gasification 311 of the undigested residue to syngas (e.g. CO, H₂) may refine out pollutants and/or corrosive compounds. Additionally, excess and/or supplemental glycerols from conversion feeds and/or TAG production (e.g. Organism H, I; FIG. 1) may be used for gasification 311. Further, the gasified residue products may be combined or supplemented with external syngas from any suitable source, without limitation. In instances, the gasified residue products may be directed to a shift reaction 317. In certain instances, the shift reaction 317 may alter the ratio and/or the concentrations of H₂O, CO, CO₂, and H₂. In certain instances, the concentrations of CO₂ and H₂ in the gasified residue products are increased by the shift reaction 317. In nonlimiting examples, a shift reaction 317 comprises a water-gas shift reaction.

The CO, CO₂ and H₂ gas streams from the shift reaction 317 may be used for any process known to a skilled artisan. Because the shift reaction is exothermic, the waste heat or thermal energy produced may be recovered. Without limitation by theory, the recoverable thermal energy may be used for generating electricity 313. Alternatively, the thermal energy may used in other parts of the process 300.

In additional embodiments, the gases produced during fermentation 310 comprise a mixture of ammonia (NH₃), carbon dioxide (CO₂), and hydrogen (H₂). Recovery and redirection of fermentation gases 312 recaptures and recycles these and other gases through process 300. For example, NH₃ is recovered during a packed bed reaction with CO₂. The recovered NH₃ is converted to ammonium bicarbonate (NH₄HCO₃) for recycle to fermentation 310 (e.g. for pH control) and/or incorporation in the acids/salts stream for conversion 350.

In embodiments, gas mixtures comprising H₂ may be recovered from the shift reactions 317, syngas processes 311, and fermentation 310. Additionally, any supplemental source of H₂ may be connected to process 300. In instances, supplemental sources of synthesis gas and hydrogen, such as reformed natural gas or electrolyzed water, may feed the process 300 and H₂ purification 316. The H₂ containing gas mixtures may be directed to further H₂ purification 316. In embodiments, purification 316 generates pure or nearly pure H₂ from syngas, gasified residue products, and supplemental streams without limitation. In certain instances, purification 316 may comprise pressure swing adsorption, where the CO₂ and H₂ are separated after a shift reaction 317. The CO₂ and other gases may be vented to atmosphere or used in external processes, such as but not limited to algae culturing. Purified H₂ may be used in conversion 350 and/or processing 370. And in certain circumstances, the entire process 300 may run on supplemental sources of synthesis gas or hydrogen. In these embodiments the process 300 is an example of gas-to-liquids conversion.

In regards to glycerol and TAG, supplemental glycerol may be used during conversion 350 for certain organisms (e.g. Organism H, I; FIG. 1). Additionally, glycerol may be synthesized during conversion 350 (e.g. Organism I; FIG. 1). The excess glycerol resulting from supplemental feeds and synthesis during conversion may be recovered during processing 370. Supplemented and/or recycled glycerol may be fed to gasification 311 for conversion to syngas, and other gases, returned for conversion 350 for heterotrophic metabolization to produce more TAG, returned to fermentation 310, or combinations thereof.

### Third Integrated Process

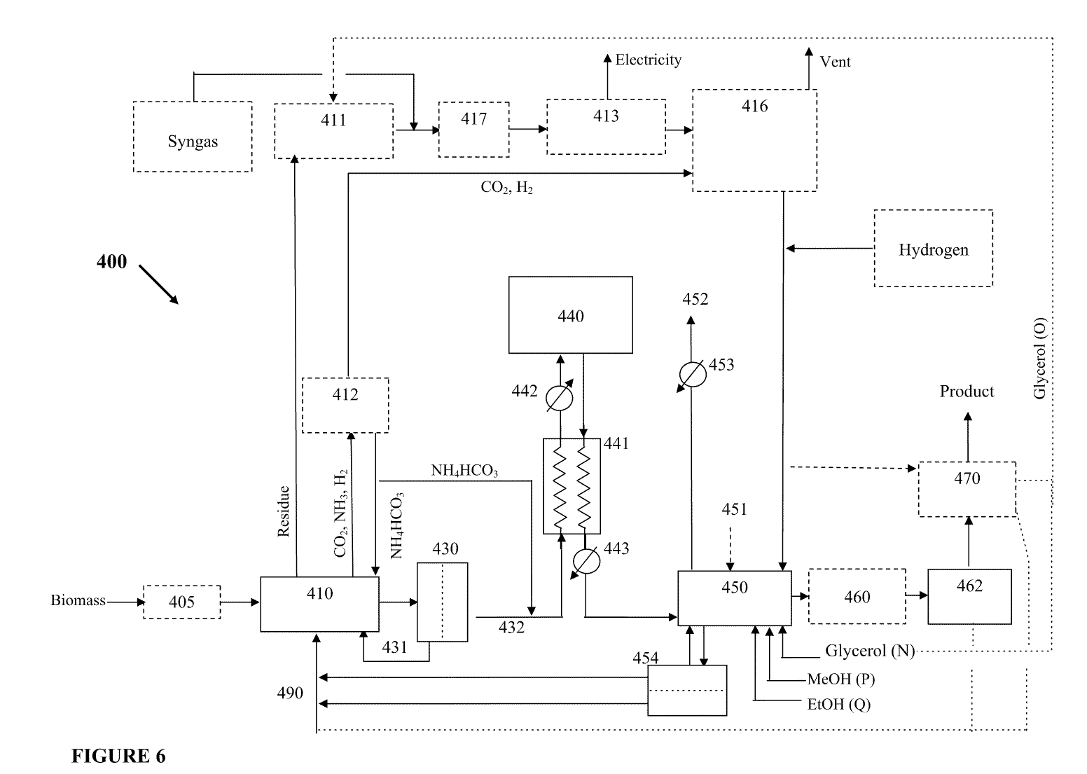

Referring now to FIG. 6 illustrating a third embodiment of the process generally shown in FIG. 3, the process 400 for converting biomass to hydrocarbon products, the process generally comprises the steps of fermentation 410, withdrawing an acids/salts solution 430, converting 450 the acids/salts solution to conversion products, processing 470 the conversion products to hydrocarbon products, and recycling 490 a portion of the products. Additionally, the process 400 may include gasification 411 of undigested fermenter residues, ammonia recovery 412, and hydrogen (H₂) purification 416.

The process 400 is configured similarly to the process 300 previously disclosed and illustrated in FIG. 5. More specifically, the process 400 is for the integration of heterotrophic organisms that require, prefer, or optionally use nitrates (NO₃⁻) as an electron receptor after NADPH regeneration. Exemplary organisms M through R may be found in FIG. 1. In embodiments, the organisms M through R in FIG. 1 comprise heterotrophic organisms that convert fermentation products, including mixed acids and salts, using the enzyme hydrogen dehydrogenase for regeneration of NADPH. Further, as shown in FIG. 1 the organisms M through R are aerobic or anaerobic, in that they may or may not utilize oxygen as an electron acceptor.

In embodiments, the process 400 includes the same or substantially similar steps as process 300 illustrated in FIG. 5 and discussed previously. More specifically, process 400 includes biomass, which is introduced to a fermentor for the process of fermentation 410. In embodiments, biomass such as the nonlimiting examples, municipal solids waste, farm waste, lignocellulosic/starchy crops, or combinations thereof, are digested during fermentation 410. Optionally, the biomass is pretreated 405, by any method, prior to fermentation 410 to reduce or degrade lignin in high lignin content biomass. Fermentation 410 conditions favor the production of mixed acids and acid salts in the fermentation broth.

In embodiments, the fermentation broth comprising the mixed acids/salts is separated 430, and solids 431 are returned to the fermentation 410. The remaining non-sterile liquids comprising acids/salts 432 are removed from separation 430 for sterilization 440. Sterilization 440 comprises elevating the temperature of the fermentation broth to above about 100° C.; alternatively, to above 110° C.; and in certain instances over about 140° C., with steam 442 for at least about 3 minutes; alternatively, for at least about 5 minutes; and alternatively, for at least about 10 minutes. Additionally, in order to conserve, reuse, or recycle thermal energy within process 400, heat exchange 441 between the non-sterile fermentation broth 432, the sterilized acids/salts from sterilization 440, water from cooling 443 and steam 442 may be implemented as previously described. In embodiments, the cooled, sterilized acids/salts are directed to conversion 450.

In the present process 400, the conversion 450 is a heterotrophic conversion. Referring to FIG. 1 in relation to the process 400 shown in FIG. 6, the heterotrophic organisms include the metabolic configurations of M through R for the conversion of the acids/salts. Organisms M through R convert the acids/salts into hydrocarbon and/or hydrocarbon-like products by aerobic or anaerobic biosynthetic pathways. In contrast to process 300, process 400 utilizes a nitrate (NO₃⁻) supplement for conversion. Without limitation by theory, the microorganisms M through R utilize the nitrate or nitrates (NO₃⁻) as the electron acceptor after hydrogen dehydrogenase NADPH and ATP regeneration. As such, conversion 450 comprises introducing different reactants for conversion 450, as compared to process 300. Non-limiting examples of additional reactants include hydrogen, glycerol (which can be processed by Organism N), methanol (which is processed by Organism P), or ethanol (which can be processed by Organism Q). In still other embodiments, conversion 450 comprises venting or releasing waste gases 452 such as CO, CO₂ or N₂. However, to avoid losing the volatile reactants or the nitrates, cooling and condensing the vented gases 453 may be suitable. Additionally, conversion 450 may further require cooling or heating the conversion reaction to improve the conversion efficiency, conversion rate, reactant recovery, or optimize conditions for the microorganisms.

The conversion 450 forms hydrocarbon and/or hydrocarbon-like products such as WE, TAG, FAME, FAEE, PHAs, without limitations, alcohols, ketones, aldehydes, and combinations thereof, as described in detail hereinabove. In embodiments, the hydrocarbons and/or hydrocarbon-like products may be externalized as extracellular matrix molecules or as extracellular excretions that are easily separated at 454. In alternate embodiments, the hydrocarbons and/or hydrocarbon-like products are intracellular molecules, such that lysing 460 and recovering 462 are utilized. Lysing 460 and recovering 462 remove the hydrocarbons and/or hydrocarbon-like molecules from the intracellular matrix and separate them from other immiscible and/or hydrophobic cellular components. The other cellular components are optionally recycled 490 for fermentation 410. In instances the hydrocarbon and/or hydrocarbon-like molecules may be immiscible, float to the surface of aqueous solutions, and are skimmed and/or decanted off for processing 470.

Without limitation, processing 470 may comprise the synthesis of chemicals, solvents, or hydrocarbon fuels that are compatible with the present fuel infrastructure. In the nonlimiting examples the WE, TAG, FAME, FAEE, and PHAs previously discussed herein, are directed through processing that may comprise transesterification (e.g. TAG), hydrogenation, decarboxylation, isomerization, cleaving, crosslinking, and other hydrocarbon reactions, such as refining, cracking, alkylating, polymerizing, and separating. The processing 470 of the hydrocarbon and/or hydrocarbon-like molecules may further comprise incorporation of hydrogen (H₂) from any source, including additional methods and processes.

The process 400 may integrate other methods and processes, including the nonlimiting examples gasification 411, ammonia recovery 412, and hydrogen purification 416. Further, the process 400 may directly or indirectly supplement the production of electricity 413 by the formation of syngas, hydrogen, and the recovery of thermal energy therefrom.

In embodiments, undigested residues from the fermentation 410 are gasified 411 as described previously. Additionally, excess and/or supplemental glycerols from conversion feeds and/or TAG production (e.g. Organism N, O; FIG. 1) may be used for gasification 411. In regards to glycerol and TAG, supplemental glycerol may be used during conversion 450 for certain organisms (e.g. Organism N, O; FIG. 1) or glycerol may be synthesized during conversion 450 (e.g. Organism O; FIG. 1). The excess glycerol resulting from supplemental feeds and synthesis during conversion may be used for gasification 411 or further fermentation 410. In instances, the gasified residue and/or glycerol products may be directed to a shift reaction 417 for conversion of the CO, CO₂ and H₂ containing gas streams to CO₂ and H₂ rich gas streams. The CO₂ and H₂ gas streams from the shift reaction may be used to recover thermal energy, produce electricity, or directed to syngas processes, without limitation. The recovered thermal energy may also be used in other parts of the process 400.

In additional embodiments, the gases produced during fermentation 410 comprise a mixture of ammonia (NH₃), carbon dioxide (CO₂), and hydrogen (H₂). Recovery and redirection of fermentation gases 412 recycles these and other gases through process 400. In embodiments, gas mixtures comprising H₂ recovered from the shift reactions 417, gasification processes 411, fermentation 410, and supplemental sources of H₂ may be used throughout the process 400. In certain embodiments, the H₂ containing gas mixtures may be directed to a H₂ purification process 416, such as pressure swing absorption. The CO₂ and other gases may be vented to atmosphere or used in external processes, such as but not limited to algae culturing. The purified H₂ may be used in conversion 450 and/or processing 470. And in certain circumstances, the entire process 400 may run on supplemental sources of synthesis gas or hydrogen. In these embodiments the process 400 is an example of gas-to-liquids conversion.

### Fourth Integrated Process

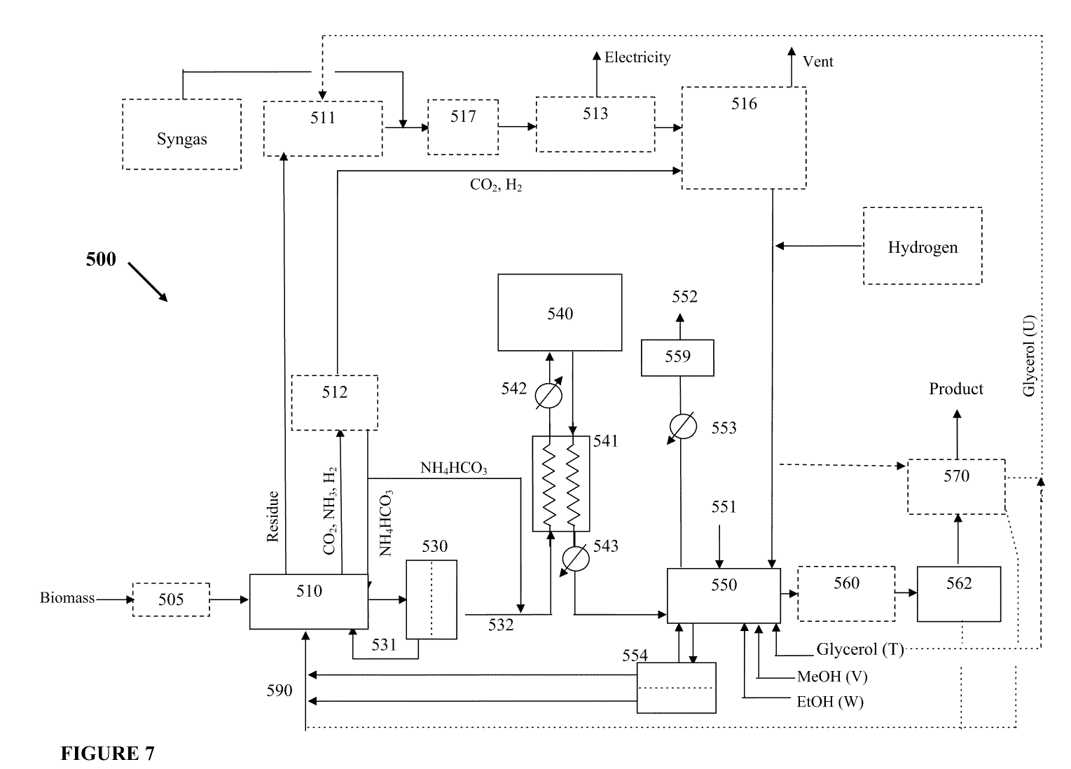

Referring now to FIG. 7 illustrating a fourth embodiment of the process generally shown in FIG. 3, the process 500 for converting biomass to hydrocarbon products, the process generally comprises the steps of fermentation 510, withdrawing 530 an acids/salts solution or broth, converting 550 the acids/salts solution to conversion products, processing 570 the conversion products to hydrocarbon products, and recycling 590 a portion of the products. Additionally, the process 500 may include gasification of undigested fermentor residues 511, ammonia recovery 512, and hydrogen (H₂) purification 516.

The process 500 is configured similarly to the process 400 previously disclosed and illustrated in FIG. 6. More specifically, the process 500 is for the integration of heterotrophic organisms that require, prefer, or optionally use sulfates (SO₄⁻²) as an electron receptor after NADPH regeneration. Exemplary organisms S through X may be found in FIG. 1. In embodiments, the organisms S through X in FIG. 1 comprise heterotrophic organisms that convert fermentation products, including mixed acids and salts, using the enzyme hydrogen dehydrogenase for regeneration of NADPH. Further, as shown in FIG. 1 the organisms S through X may by aerobic and/or anaerobic, in that they may or may not utilize oxygen as an electron acceptor.

In embodiments, the process 500 includes the same or substantially similar steps as process 400 illustrated in FIG. 6 and discussed previously. More specifically, process 500 includes biomass, which is introduced to a fermentor for the process of fermentation 510. In embodiments, biomass such as the nonlimiting examples, municipal solids waste, farm waste, lignocellulosic/starchy crops, or combinations thereof, are digested during fermentation 510. Optionally, the biomass is pretreated 505, by any method, prior to fermentation 510 to reduce or degrade lignin in high lignin content biomass. Fermentation 510 conditions favor the production of mixed acids and acid salts in the fermentation broth.

In embodiments, the fermentation broth comprising the mixed acids/salts is separated 530, and solids 531 are returned to the fermentation 510. The remaining non-sterile liquids comprising acids/salts 532 are removed by separation 530 for sterilization 540. Sterilization 540 comprises elevating the temperature of the fermentation broth to above about 100° C.; alternatively, to above 110° C.; and in certain instances over about 140° C., with steam 542 for at least about 3 minutes; alternatively, for at least about 5 minutes; and alternatively, for at least about 10 minutes. Additionally, in order to conserve, reuse, or recycle thermal energy within process 500, heat exchange 541 between the non-sterile fermentation broth 532, the sterilized acids/salts from sterilization 540, water from cooling 543 and steam 542 may be implemented as previously described. In embodiments, the cooled, sterilized acids/salts are directed to conversion 550.

In the present process 500, the conversion 550 is a heterotrophic conversion. Referring to FIG. 1 in relation to the process 500 shown in FIG. 7, the heterotrophic organisms include the metabolic configurations of S through X for the conversion of the acids/salts. Organisms S through X convert the acids/salts into hydrocarbon and/or hydrocarbon-like products by aerobic or anaerobic biosynthetic paths, and may or may not require O₂ as an electron acceptor. In contrast to process 400, process 500 utilizes sulfates (SO₄⁻²) for conversion. Without limitation by theory, the microorganisms S through X utilize the sulfate(s) (SO₄⁻²) as the electron acceptor after hydrogen dehydrogenase NADPH and ATP regeneration. Conversion 550 also comprises introducing different reactants for conversion 550, as compared to process 400. Non-limiting examples of additional reactants include hydrogen, glycerol (which can be processed by Organism T), methanol (which can be process by Organism V), or ethanol (which can be processed by Organism W). In still other embodiments, conversion 550 comprises venting or releasing waste gases 552 such as CO, CO₂, or N₂. However, to avoid losing the volatile reactants or the sulfates, cooling and condensing the vented gases 553 may be suitable. However as sulfates are reduced to H₂S, a clean-up process or sulfur recovery process 559 may be used during venting 552. Without limitation by theory, a clean-up process or sulfur recovery process 559 prevents release of H₂S gas to atmosphere. Additionally, conversion 550 may further require cooling or heating the conversion reaction to improve the conversion efficiency, conversion rate, reactant recovery, or optimize conditions for the microorganisms.

The conversion 550 forms hydrocarbon and/or hydrocarbon-like products such as WE, TAG, FAME, FAEE, PHAs, alcohols, ketones, aldehydes, and combinations thereof without limitations, as described in detail hereinabove. In embodiments, the hydrocarbons and/or hydrocarbon-like products may be externalized as extracellular matrix molecules or as extracellular secretions that are easily separated at 554. In alternate embodiments, the hydrocarbons and/or hydrocarbon-like products are intracellular molecules, such that cellular lysing 560 and recovery 562 are utilized. Lysing 560 removes the hydrocarbons and/or hydrocarbon-like molecules from the cells' intracellular matrix. Recovery 562 removes the hydrocarbons and/or hydrocarbon-like molecules from other immiscible or hydrophobic intracellular components. The other intracellular components are optionally recycled 590 for fermentation 510. In instances the hydrocarbon and/or hydrocarbon-like molecules may be immiscible, and float to the surface of aqueous solutions for skimming or decanting for processing 570.

Without limitation, processing 570 may comprise the synthesis of chemicals, solvents, or hydrocarbon fuels that are compatible with the present fuel infrastructure. Processing 570 may comprise transesterification (e.g. TAG), hydrogenation, decarboxylation, isomerization, cleaving, crosslinking, and other hydrocarbon reactions, such as refining, cracking, alkylating, polymerizing, and separating. The processing 570 of the hydrocarbon and/or hydrocarbon-like molecules may further comprise incorporation of hydrogen (H₂) from any source, including additional methods and processes

The process 500 may integrate other methods and processes, including the nonlimiting examples gasification 511, ammonia recovery 512, gas shift 517 and hydrogen purification 516. Further, the process 500 may directly or indirectly supplement the production of electricity 513 by the formation of syngas, hydrogen, and the recovery of thermal energy therefrom. In embodiments, the integrated methods and processes may be used to recover thermal energy or produce electricity for use throughout process 500. The integrated methods and processes may be directed to the production of H₂ and/or syngas for use throughout the process as previously described. In embodiments, the integrated methods comprise H₂ recovery, generation, and recycle processes.

In embodiments, gas mixtures comprising H₂ recovered and/or recycled from the shift reactions 517, gasification processes 511 (e.g. syngas production), fermentation 510, and supplemental sources of H₂ may be used throughout the process 500. In certain embodiments, the H₂ containing gas mixtures may be directed to a H₂ purification process 516 prior to being used elsewhere. The purified H₂ may be used in conversion 550 and/or processing 570. And in certain circumstances, the entire process 500 may run on supplemental sources of synthesis gas or hydrogen. In these embodiments the process 500 is an example of gas-to-liquids conversion.

### Fifth Integrated Process

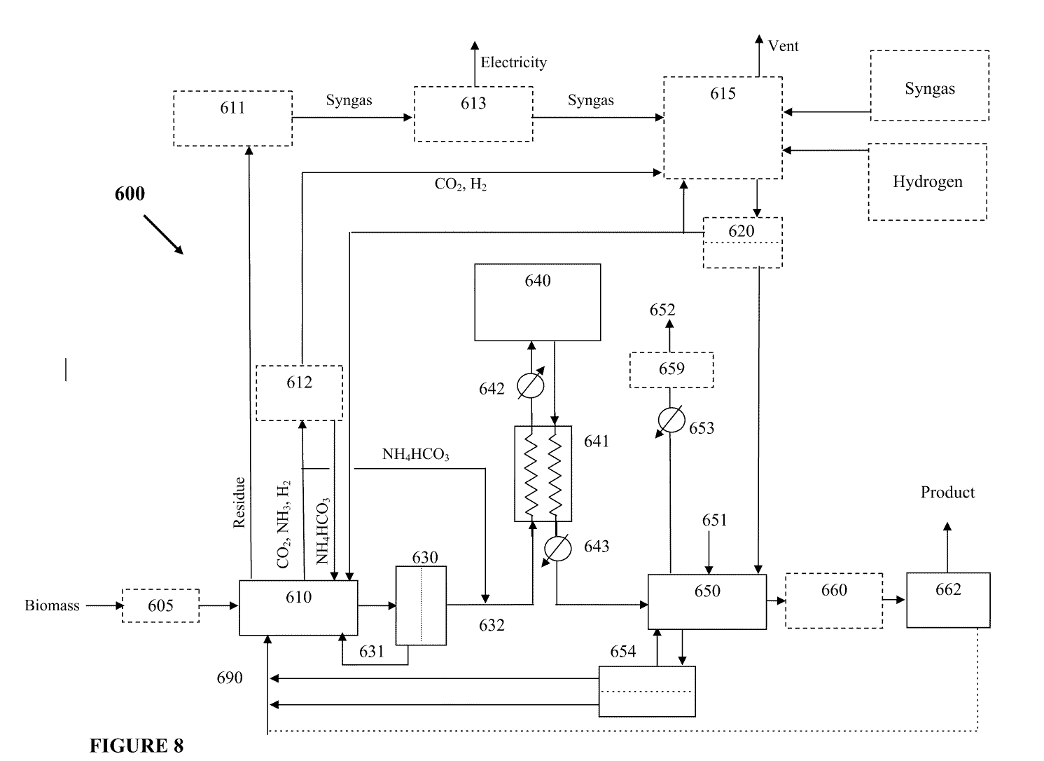

Referring now to FIG. 8 illustrating an embodiment of the process generally shown in FIG. 3, the process 600 for converting biomass to hydrocarbon products, the process generally comprises the steps of fermentation 610, withdrawing 630 an acids/salts solution, converting 650 the acids/salts solution to conversion products in a conversion process, recovering 662 the conversion products, which are hydrocarbons, and recycling 690 a portion of the products.

In embodiments, the process 600 includes the same or substantially similar steps as processes 200 illustrated in FIG. 4 and discussed previously. More specifically, process 600 includes biomass, which is introduced to a fermentor for the process of fermentation 610. In embodiments, biomass such as the non-limiting examples, municipal solids waste, farm waste, lignocellulosic/starchy crops, or combinations thereof, are digested during fermentation 610. Optionally, the biomass is pretreated 605, by any method, prior to fermentation 610 to reduce or degrade lignin in high lignin content biomass. Fermentation 610 conditions favor the production of mixed acids and acid salts in the fermentation broth.

In embodiments, the fermentation broth comprising the mixed acids/salts is separated 630, and solids 631 are returned to the fermentation 610. The remaining non-sterile liquids comprising acids/salts are removed 632 from separation 630 for sterilization 640. Sterilization 640 comprises elevating the temperature of the fermentation broth to above about 100° C.; alternatively, to above 110° C.; and in certain instances over about 140° C., with steam 642 for at least about 3 minutes; alternatively, for at least about 5 minutes; and alternatively, for at least about 10 minutes. Additionally, in order to conserve, reuse, or recycle thermal energy within process 600, heat exchange 641 between the non-sterile fermentation broth 632, the sterilized acids/salts from sterilization 640, water from cooling 643 and steam 642 may be implemented as previously described. In embodiments, the cooled, sterilized acids/salts are directed to conversion 650.

In embodiments, process 600 is arranged to integrate a fermentor with at least one of the biosynthetic organisms shown in FIG. 1. Conversion 650 is an aerobic, heterotrophic conversion. Referring to FIG. 1 in relation to process 600 of FIG. 8, the heterotrophic organisms include the metabolic configurations of A through L for the conversion of the acids/salts, except that they produce hydrocarbon molecules directly as opposed to hydrocarbon-like intermediates. Organisms A through F convert fermentation products, including mixed acids/salts to hydrocarbons, using the TCA cycle for regeneration of NADPH. Organisms G through L convert the acids/salts into hydrocarbon and/or hydrocarbon-like products by aerobic biosynthetic paths, using introduced air or oxygen (O₂) 351 as the electron acceptor after hydrogen dehydrogenase NADPH regeneration and ATP regeneration. However, only process 600 is arranged for the direct synthesis of hydrocarbons during conversion 650

In embodiments, air or oxygen (O₂) is introduced 651 during conversion 650 to act as the electron acceptor at the end of the TCA cycle mediated ATP regeneration for organisms with metabolic configuration similar to A through F. The air or oxygen (O₂) is introduced 651 during conversion 650 to act as the electron acceptor for hydrogen dehydrogenase mediated ATP regeneration for organisms with metabolic configurations similar to G through L. Nonetheless, sulfate and nitrate may also be used as alternate electron acceptors.

In still other embodiments, conversion 650 comprises venting or releasing waste gases 652 such as CO, CO₂. However, measures may be taken to avoid losing the volatile reactants in the conversion. In certain instances, cooling and condensing 653 the gases being vented is suitable to recover volatile reactants. Conversion may further require cooling or heating the conversion reaction to improve the conversion efficiency, conversion rate, reactant recovery, or optimize conditions for the microorganisms. If sulfate is used as an additional electron acceptor, then a hydrogen sulfide cleaning unit 659 would be needed.

The conversion process 650 may include selectively separating microorganisms 654 for recycling within the conversion process 650. In embodiments where microorganisms produce hydrocarbons as extracellular matrix molecules or extracellular secretions does not require the lysis 660 of the microorganisms. As understood by a skilled artisan, there are many ways to recover the hydrocarbons. As hydrocarbons tend to be immiscible, they therefore float to or on the surface of aqueous solutions. In embodiments, the extracellular hydrocarbons may be decanted or skimmed in recovery step 662, without limitation.

In embodiments where the microorganisms produce intracellular hydrocarbons and/or hydrocarbon-like molecules, the microorganisms are subjected to lysing 660. Lysing 660 further comprises concentrating the microorganism cell mass and any process suitable for rupturing a cell membrane. Non-limiting examples of lysing 660 including centrifuging, osmotic shocking, supercritical fluid extraction, solvent extraction, cold pressing, shearing, homogenizing, blending, milling, sonication, or other techniques. As previously described, there are many ways to recover the hydrocarbons from intracellular proteins, and any method is suitable for directing the hydrocarbons to recovery 662.

Recovery 662 comprises separation, purification, and refining of hydrocarbons from conversion 650. In embodiments, recovery 662 may be used for cracking, upgrading, or other refinery process without limitation. As the hydrocarbons in process 600 were directly produced by the microorganisms during conversion 650, they may be ready for immediate sale or implementation into other process. In non-limiting examples, the hydrocarbons may be liquid fuels, solvents, or other chemical commodities.

The process 600 may integrate other methods and processes, including the non-limiting examples gasification 611, ammonia recovery 612, gas shift 617 and hydrogen purification 616. Further, the process 600 may directly or indirectly supplement the production of electricity 613 by the formation of syngas, hydrogen, and the recovery of thermal energy therefrom. In embodiments, the integrated methods and processes may be used to recover thermal energy or produce electricity for use throughout process 600. The integrated methods and processes may be directed to the production of H₂ and/or syngas for use throughout the process as previously described. In embodiments, the integrated methods comprise H₂ recovery, generation, and recycle processes.

### Sixth Integrated Process

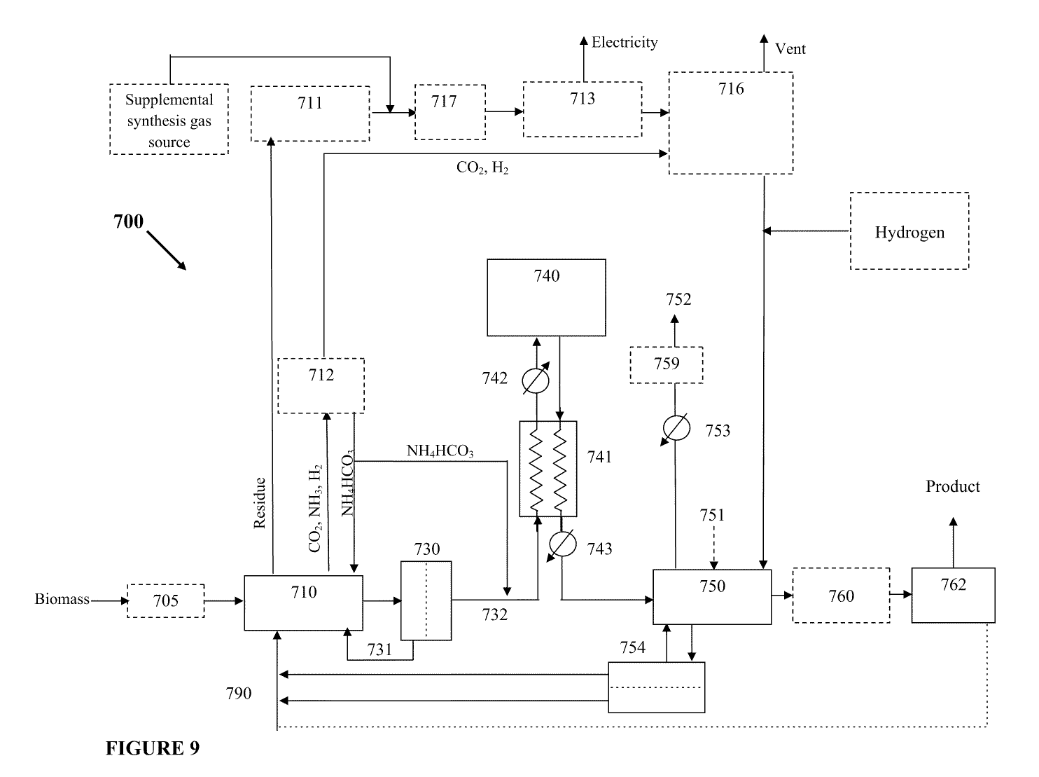

Referring now to FIG. 9 illustrating a sixth embodiment of the process generally shown in FIG. 3, the process 700 for converting biomass to hydrocarbon products, the process generally comprises the steps of fermentation 710, withdrawing 730 an acids/salts solution, converting 750 the acids/salts solution to conversion products in conversion process, recovering 762 the conversion products to hydrocarbon products in recovery process, and recycling a portion of the products 790. Additionally, the process 700 may include gasification of undigested fermentor residues 711, ammonia recovery 712, and hydrogen (H₂) purification 716.

Referring to FIG. 6, FIG. 7 and FIG. 9, process 700 is configured identical to the process 300, 400 and process 500 until conversion 750 and recovering 762. In embodiments, all three processes 400, 500, and 700 are arranged to integrate a fermentor with at least one of the biosynthetic organisms shown in FIG. 1. However, process 700 is configured for the direct synthesis of hydrocarbons during conversion 750. More specifically, the processes 400, 500, and 700 integrate organisms with metabolic configuration of organisms M through X. These are heterotrophic organisms that convert fermentation products, including mixed acids/salts to hydrocarbons, using the hydrogen dehydrogenase for regeneration of NADPH, and utilize nitrates (NO₃⁻) and/or sulfates (SO₄²) as an electron receptor.

In embodiments, the fermentation broth comprising the mixed acids/salts is separated 730, and solids 731 are returned to the fermentation 710. The remaining non-sterile liquids comprising acids/salts are removed 732 from separation 730 for sterilization 740. Sterilization 740 comprises elevating the temperature of the fermentation broth to above about 100° C.; alternatively, to above 110° C.; and in certain instances over about 140° C., with steam 642 for at least about 3 minutes; alternatively, for at least about 5 minutes; and alternatively, for at least about 10 minutes. Additionally, in order to conserve, reuse, or recycle thermal energy within process 700, heat exchange 741 between the non-sterile fermentation broth 732, the sterilized acids/salts from sterilization 740, water from cooling 743 and steam 742 may be implemented as previously described. In embodiments, the cooled, sterilized acids/salts are directed to conversion 750.

In the present process 700, the conversion 750 is a heterotrophic conversion. Referring to FIG. 1 in relation to the process 700 shown in FIG. 9, the heterotrophic organisms include the metabolic configurations of M through X for the conversion of the acids/salts but they produce hydrocarbons directly rather than hydrocarbon-like intermediates as in FIG. 1. More specifically, organisms with metabolic configuration similar to Organisms M through R convert the acids/salts into hydrocarbon products by aerobic or anaerobic biosynthetic paths. The microorganisms M through R utilize the nitrate or nitrates (NO₃⁻) as the electron acceptor after hydrogen dehydrogenase NADPH and ATP regeneration. Without limitation by theory, the microorganisms with metabolic configuration similar to that of M through R utilize the sulfate(s) (SO₄²) as the electron acceptor after hydrogen dehydrogenase mediated NADPH and ATP regeneration. Also, organisms with metabolic configuration S through X convert the acids/salts into hydrocarbon and products by aerobic or anaerobic biosynthetic paths, as they do not require O₂ as an electron acceptor. As such, organisms with metabolic configuration similar to S through X in FIG. 1 utilize sulfates (SO₄⁻²) for conversion.

In embodiments, conversion 750 comprises venting or releasing waste gases such as CO, CO₂, or H₂. However, to avoid losing the volatile reactants, nitrates, or sulfates, cooling and condensing the vented gases 753 may be suitable. In embodiments, as sulfates are reduced to H₂S, a clean-up process or sulfur recovery process 759 may be used to during venting 752. Without limitation by theory, a clean-up process or sulfur recovery process 759 prevents release of H₂S gas to atmosphere. Additionally, conversion 750 may further require cooling or heating the conversion reaction to improve the conversion efficiency, conversion rate, reactant recovery, or optimize conditions for the microorganisms.

The conversion process 750 may include selectively separating microorganisms 754 for recycling within the conversion process 750. In embodiments where microorganisms produce hydrocarbons as extracellular matrix molecules or extracellular excretions, the separation 754 does not require the lysis of the microorganisms. In embodiments where the microorganisms produce intracellular hydrocarbons and/or hydrocarbon-like molecules, the microorganisms are subjected to lysing 760. Lysing 760 further comprises concentrating the microorganism cell mass and any process suitable for rupturing a cell membrane. Non-limiting examples of lysing 760 including centrifuging, osmotic shocking, supercritical fluid extraction, solvent extraction, cold pressing, shearing, homogenizing, blending, milling, sonication, or other techniques. As understood by a skilled artisan, there are many ways to recover the hydrocarbons. As hydrocarbons tend to be immiscible, they therefore float to or on the surface of aqueous solutions. In embodiments, the extracellular hydrocarbons may be decanted or skimmed and directed in recovery 762, without limitation.

Recovery 762 comprises separation, purification, and refining of hydrocarbons from conversion 750. In embodiments, recovery step 762 may be used for cracking, upgrading, or other refinery process without limitation. As the hydrocarbons in process 700 were directly produced by the microorganisms during conversion 750, they may be ready for immediate sale or implementation into other process. In non-limiting examples, the hydrocarbons may be liquid fuels, solvents, or other chemical commodities.

The process 700 may integrate other methods and processes, including the non-limiting examples gasification 711, ammonia recovery 712, gas shift 717 and hydrogen purification 716. Further, the process 700 may directly or indirectly supplement the production of electricity 713 by the formation of syngas, hydrogen, and the recovery of thermal energy therefrom. In embodiments, the integrated methods and processes may be used to recover thermal energy or produce electricity for use throughout process 700. The integrated methods and processes may be directed to the production of H₂ and/or syngas for use throughout the process as previously described. In embodiments, the integrated methods comprise H₂ recovery, generation, and recycle processes.

### Additional Heterotrophic Integration

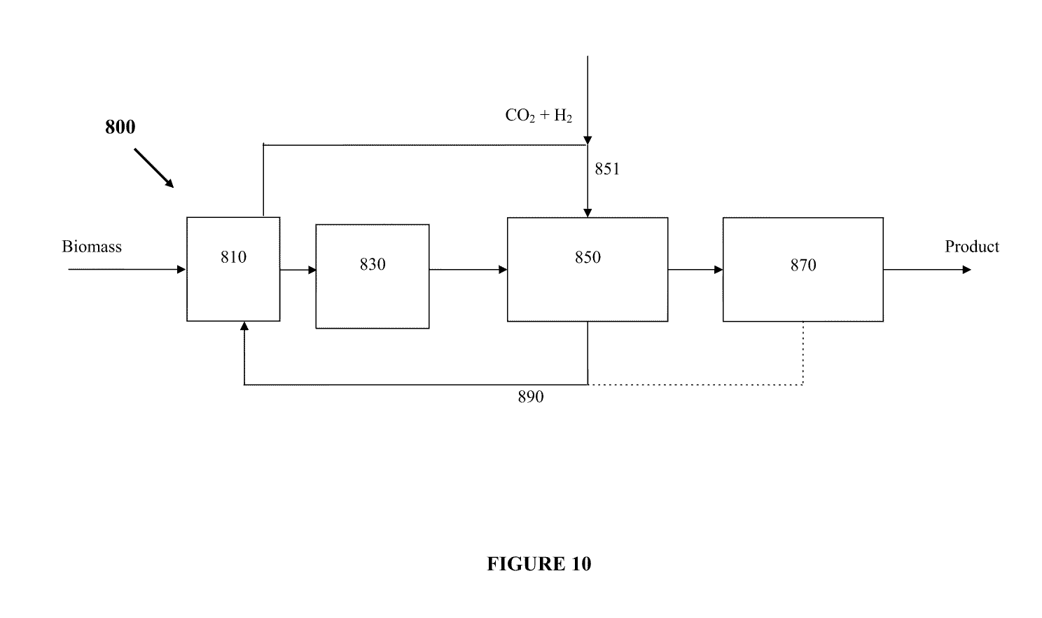

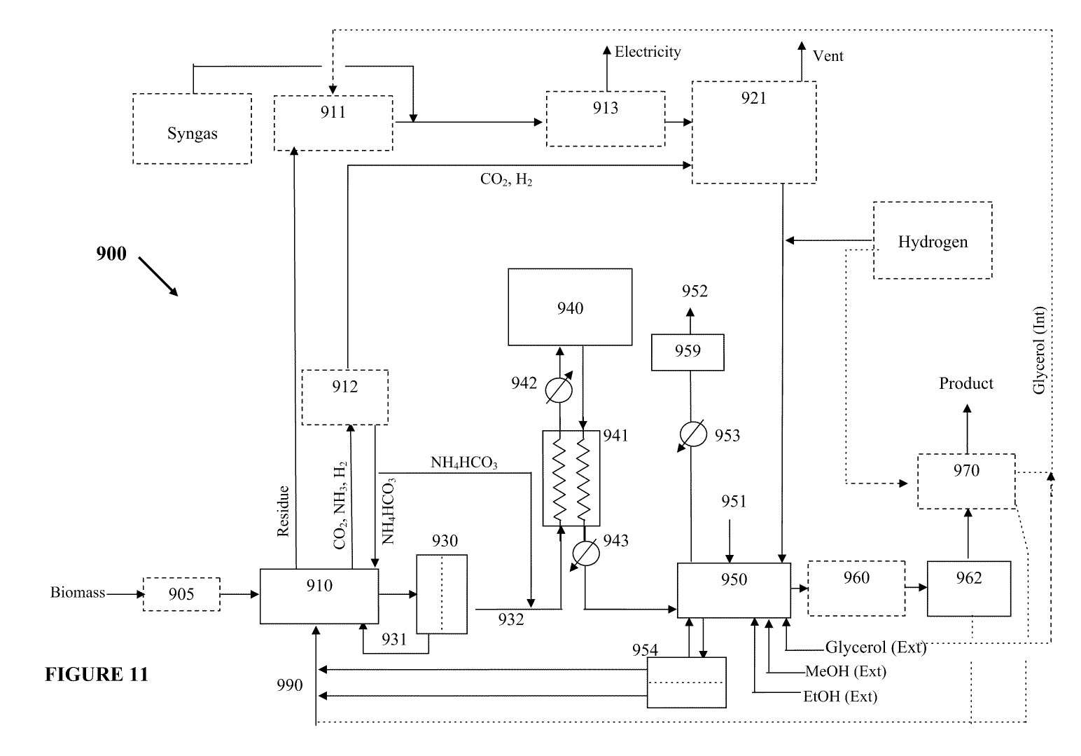

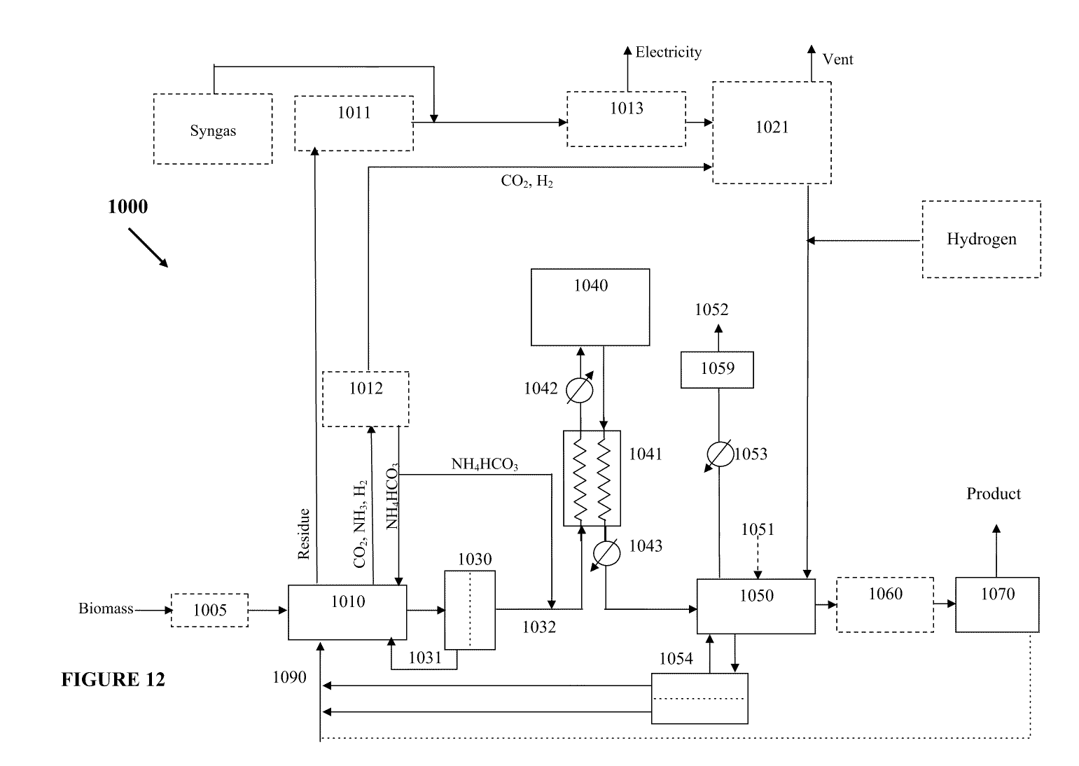

Processes 600 and 700 include direct conversion 650, 750 of salts/acids to hydrocarbons. As the hydrocarbons may require minimal post synthesis processing to be suitable for sale, the integration of additional processes is expanded. In non-limiting examples, the additional processes comprising gasification 611, 711, ammonia recovery 612, 712 thermal energy recovery/electricity generation 613, 713, other microorganism mediated processes 615, H₂ purification 716, and/or shift reactions 717 may be utilized in either process 800 in FIG. 10, 600 of FIG. 8, 700 of FIG. 9, and more specifically in process 900 and 1000 in FIGS. 11 and 12, respectively. Without limitation by theory, integration of other steps, feeds, and processes into the processes 800, and specifically 900 and 1000 reduces capital cost, improves raw material usage, and improves operational efficiency, and operation flexibility for the integrated mixed acid fermentation and microorganism mediated hydrocarbon production process. In certain embodiments, these additional methods and processes may directly or indirectly generate electricity. Additionally, the recovered 612, 712 NH₃ may be converted to ammonium bicarbonate (NH₄HCO₃) for recycle to fermentation 610, 710 (e.g. for pH control) and/or incorporation in the acids/salts stream for conversion 650, 750.

### Chemo-Mixotrophic Methods

Referring now to FIG. 10, illustrating a block flow diagram for a general process 800 for converting acids, acid salts, and combinations thereof to chemical products or hydrocarbon products. The process 800 includes hydrocarbons, hydrocarbon-like molecules, or combinations thereof produced by chemo-mixotrophic organisms. The process 800 comprises introducing biomass to a fermenter 810, separating liquid fermenter products 830, converting the liquid fermenter products to form conversion products 850, and processing the conversion products into chemical products or hydrocarbon products 870. Some materials are recycled 890 back through the process 800 by re-introduction to the fermentation step 810 from the conversion 850 and processing 870 steps.

In embodiments FIG. 10 is a process flow diagram for the integration of anaerobic fermentation 810 and mixotrophic conversion 850. Fermentation 810 generally comprises a variety of anaerobic bacteria converting biomass into mixed acids or acid salts, herein acids/salts. Without limitation by theory, suitable biomass comprises any biological material that ferments to form acids and acid salts in solution. The resulting acid/salt solution is separated 830 and fed to mixotrophic conversion 850. During the conversion 850, at least one mixotrophic organism converts the acids/salts solution into hydrocarbons or hydrocarbon-like conversion products, in the presence of inorganic energy sources. Inorganic energy sources may comprise hydrogen, carbon dioxide, and/or other inorganic molecules. The conversion products are processed 870 into biofuels, biochemical products, or other chemical commodities. Water, intact and lysed cells, macromolecules, byproducts, and un-reacted acids/salts from the chemo-mixotrophic conversion 850 and processing 870 may be redirected for recycling 890 to recover acids/salts, by-products, biofuels, biochemical, or other components. In certain instances, the chemo-mixotrophic conversion step 850 returns organic materials to the recycling step 890 for fermentation 810 and the processing step 870 returns water and dilute solutions. Alternatively, only one process chosen from the conversion step 850 and the processing step 870 feeds the recycling step 890.

In the following discussion and illustrations of various embodiments of the general process discussed hereinabove, similar processes and pathways are noted by similar reference numerals. For example, the step of fermentation 110 may be indicated as 210, 310, 410, etc in the subsequent figures and discussion. Additionally, the step of conversion 150 may be indicated as 250, 350, 450, etc. While the general steps may be related, the specific properties, reactions, and products of the general steps may differ, and therefore should not be limited to any particular embodiment described preceding discussion, or shown in a preceding illustration, but only to the description that accompanies it.

### Seventh Integrated Process

Referring now to FIG. 11 illustrating an embodiment of the process generally shown in FIG. 10, the process 900 for converting biomass to hydrocarbon products, the process generally comprises the steps of fermentation 910, separating 930 an acids/salts solution, converting 950 the acids/salts solution to conversion products, processing 970 the conversion products to hydrocarbon products, and recycling 990 a portion of the by-products.

In embodiments, the process 900 is configured to integrate a fermentor with microorganisms capable of biosynthesis of hydrocarbons and/or hydrocarbon-like molecules. More specifically, the process 900 is for the integration of mixotrophic organisms into a mixed-acid fermentation. Mixotrophic organisms convert fermentation products, including mixed acids and salts into hydrocarbons and/or hydrocarbon-like molecules in the presence of inorganic carbon and/or energy sources. Further, the mixotrophic organisms may utilize any electron acceptor to regenerate ATP, and in non-limiting examples, oxygen, nitrates, sulfates,

The biomass may be pretreated 905 prior to fermentation 910. In embodiments, the biomass with high lignin content interferes with the mixed acid fermentation by binding and hindering microorganism from accessing and digesting the polymeric sugars, such as cellulose and hemicellulose. Non-limiting examples of potential pretreatment processes include sulfuric acid pretreatment, hot water pretreatment, steam pretreatment or autoclaving, ammonia pretreatment, ammonia-fiber expansion (AFEX), and lime pretreatment. Additional pretreatment processes may be found for example in U.S. Pat. No. 5,865,898, U.S. Pat. No. 5,693,296, or U.S. Pat. No. 6,262,313, incorporated herein by reference, without limitation. After pretreatment 905, the pretreated biomass is subjected to mixed acid fermentation 910.

The biomass is introduced in raw or pretreated to a fermentor for the process of fermentation 910. In embodiments, biomass such as the non-limiting examples, municipal solids waste, farm waste, lignocellulosic/starchy crops, or combinations thereof, are digested during fermentation 910. Fermentation 910 conditions favor the production of mixed acids/salts in the fermentation broth. In non-limiting examples, the mixed acids/salts comprise mixed carboxylic acids/salts.

In embodiments, the fermentation broth comprises a non-sterile suspension or colloid including biomass debris, suspended solids, cellular debris, microorganisms, acids/salts and other fermentation products. After fermentation 910 the fermentation broth comprising the mixed acids/salts is separated 930. In embodiments, separating 930 the fermentation broth further comprises separating the solids from the liquids. The solids including biomass debris, macroscopic suspended solids and particles may be screened, filtered, settled, centrifuged, or decanted from the unsterilized liquids including microorganisms, microscopic suspended solids, cellular debris and the acids/salts. The separated solids 931 are returned for further digestion and fermentation 910 to further acids/salts. The non-sterile liquids 932 comprising acids/salts are removed from separation 930 to sterilization 940 prior to conversion 950.

In embodiments the non-sterile liquids, comprising the acids/salts are sterilized 940 prior to conversion 950. The sterilization process 940 of the fermentation broth liquids comprises thermal, pressure, autoclaving, UV, and combinations thereof, to form a sterilized acids/salts broth. Further, the fermentation broth may be sterilized 940 in a batch process. A batch process may allow a longer residence time at the sterilization temperature. Without limitation by theory, increased residence time at the sterilization temperature kills the fermentation microorganisms in the broth and degrades enzymes and other proteins that may negatively impact the conversion of the carboxylic acids/salts in sterile conversion process 950. Alternatively, without limitation, sterilization 940 comprises a continuous flow process, such as a plug-flow reactor in a non-limiting example. Without limitation by theory, continuous flow sterilization reduces deposition or settling of microscopic suspended solids in the sterilization apparatus and agitates or homogenizes the fermentation broth.

In embodiments, sterilization 940 comprises elevating the temperature of the fermentation broth to above about 100° C.; alternatively, to above 110° C.; and in certain instances over about 140° C. The sterilization 940 further comprises heating the fermentation broth with steam 942. In certain embodiments, the fermentation broth is sterilized for at least about 3 minutes; alternatively, for at least about 5 minutes; and alternatively, for at least about 10 minutes. Alternatively, the fermentation broth is sterilized by continuously filling a sterilization reactor, sterilizing the fermentation broth, and draining the sterilized acids/salts.

To conserve, reuse, or recycle thermal energy within process 900, heat exchange 941 between the unsterile fermentation broth and the sterilized acids/salts may be implemented. Without limitation by theory, heat exchange 941 warms the non-sterile broth prior to introduction of steam 942. Warming the non-sterile broth by heat exchange 941 reduces the volume, temperature, and pressure of the steam introduction 942 needed to heat the broth to sterilization temperature. Additionally, heat exchange 941 at least partially cools the sterilized acids/salts prior to conversion 950. In embodiments, the sterilized acids/salts are further cooled 943 prior to conversion by heat exchange with water. As above, to conserve, reuse, or recycle thermal energy within process 900, the water from cooling 943, warmed by thermal energy from the sterilized acids/salts, may be used for steam introduction 942 and sterilization 940. In embodiments, the cooled, sterilized acids/salts are directed to conversion 950.

In the present process 900, the conversion 950 is a chemo-mixotrophic conversion. The conversion 950 forms hydrocarbon-like products such as WE, TAG, FAME, FAEE, PHAs, other hydrocarbon-like molecules, and combinations thereof, as described in detail hereinabove. In further embodiments, the hydrocarbon-like products may comprise hydrocarbon alcohols (e.g. hexanol), ketones, or aldehydes, without limitation. In embodiments, the hydrocarbons and/or hydrocarbon-like products may be externalized as extracellular matrix molecules or as extracellular excretions. In alternate embodiments, the hydrocarbons and/or hydrocarbon-like products are intracellular molecules.

Conversion 950 follows inorganic biosynthetic conditions and chemo-autotrophic pathways. Carbon dioxide (CO₂), carbon monoxide (CO) and hydrogen (H₂) may be introduced 951 and used during the conversion 950. In instances, the CO₂, CO, and H₂ may come from outside the process and/or from components of the system, such as without limitation, gasification 911, ammonia recovery 912, and purification 921. Additional reactants may be introduced for conversion, including from external or internal sources with respect to the process 900. Non-limiting examples of additional reactants include glycerol, methanol, or ethanol. Conversion 950 may further require cooling or heating the conversion reaction to improve the conversion efficiency, conversion rate, reactant recovery, or optimize conditions for the microorganisms.

Conversion 950 may use any electron acceptor known to a skilled artisan. As described in multiple embodiments herein, O₂, NO₃⁻, and SO₄⁻² may be suitable electron acceptors. Conversion 950 comprises venting or releasing 952 the reduced electron acceptors and waste gases such as O₂, or H₂O. Certain measures may be taken to avoid losing the volatile reactants in the conversion. In a non-limiting example, cooling and condensing 953 the gases being vented is suitable to recover volatile reactants. In embodiments, as sulfates are reduced to H₂S, a clean-up process or sulfur recovery process 959 may be used to during venting 952. Without limitation by theory, a clean-up process or sulfur recovery process 959 prevents release of H₂S gas to atmosphere.

The conversion process 950 may include selectively separating microorganisms 954 for recycling within the conversion process 950. In embodiments, microorganisms that produce hydrocarbons and/or hydrocarbon-like molecules that are extracellular matrix molecules or extracellular excretions do not require the lysis 960 of the microorganisms for separation. As understood by a skilled artisan, there are many ways to recover the hydrocarbons and/or hydrocarbon-like molecules from aqueous solutions 962. In certain instances, the hydrocarbon or hydrocarbon-like molecules tend to be immiscible and therefore float to the surface of aqueous solutions. The extracellular hydrocarbons and/or hydrocarbon-like molecules may be decanted or skimmed prior to processing 970, without limitation.

The remaining suspension comprising the mixotrophic microorganisms, unconverted acids/salts, and conversion media or liquid, is directed to separation 954. Separation 954 may comprise filtering, settling, washing, centrifuging, or other methods to remove the microorganisms from the liquid. The liquid may be a suspension comprising unconverted acids/salts, waste products, dead microorganisms, and other suspended solids, without limitation. In embodiments, the liquid is recycled 990 for supplying additional liquids and components to fermentation 910. Additionally, solids such as the unconverted acids/salts, waste products, dead microorganisms, and other suspended solids are also recycled 990 for fermentation 910. The liquids may be recycled 990 to fermentation 910 concurrently or separately from the unconverted acids/salts, waste products, dead microorganisms, and other suspended solids. In embodiments, the microorganisms may be returned for the conversion 950.

Alternatively, in embodiments where the microorganisms produce intracellular hydrocarbon and/or hydrocarbon-like molecules, the microorganisms are subjected to lysing 960. Lysing 960 further comprises concentrating the microorganism cell mass for example by centrifugation or flocculation, without limitation. Lysing 960 may comprise any process suitable for rupturing a cell membrane and solubilizing the intracellular matrix known to a skilled artisan. Non-limiting examples of lysing 960 including centrifuging, osmotic shocking, supercritical fluid extraction, solvent extraction, cold pressing, shearing, homogenizing, blending, milling, sonication, or other techniques.

In embodiments, lysing 960 the microorganisms comprises separating 962 the hydrocarbons and/or hydrocarbon-like molecules from other cellular components, comprising proteins, enzymes, membranes, nucleic acids and liquids from the lysed microorganisms. As previously described, there are many ways to recover the hydrocarbons and/or hydrocarbon-like molecules, and in instances, the hydrocarbon or hydrocarbon-like molecules tend to be immiscible and therefore float to the surface of aqueous solutions. In embodiments, the extracellular hydrocarbons and/or hydrocarbon-like molecules may be decanted or skimmed and directed to processing 970, without limitation. Alternatively, the hydrocarbon and hydrocarbon-like molecules may be aggregated with other cellular components that are immiscible or hydrophobic. As such, to separate the hydrocarbon and/or hydrocarbon-like molecules, any process known to a person of skill in the art may be used, including membrane separation, filtering, and centrifuging. The other cellular components, comprising proteins, enzymes, membranes, and liquids may be recycled 990 for fermentation 910. Intracellular liquids may be recycled 990 to fermentation 910 concurrently or separately from the other cellular components.

In embodiments, whether from extracellular production or cell lysing and recovery, the hydrocarbon and/or hydrocarbon-like molecules are directed to processing 970. Without limitation, processing 970 may chemically convert them into chemicals, solvents, or hydrocarbon fuels that are compatible with the present fuel infrastructure. In the non-limiting examples the WE, TAG, FAME, FAEE, and PHAs previously discussed herein, processing may comprise transesterification (e.g. TAG), hydrogenation, decarboxylation, isomerization, cleaving, cross-linking, and other hydrocarbon reactions, such as refining, cracking, alkylating, polymerizing, and separating. The processing 970 of the hydrocarbon and/or hydrocarbon-like molecules may further comprise incorporation of hydrogen (H₂).

The process 900 may integrate other methods and processes. Without limitation by theory, integration of other steps, feeds, and processes into the process 900 reduces capital cost, improves raw material usage, and improves operational efficiency and flexibility. Non-limiting process examples include gasification 911, ammonia recovery 912, and electricity generation 913. In certain embodiments, the undigested residue from fermentation 910 and excess glycerols, from conversion feeds (i.e. external) and conversion (i.e. internal) sources may be used for gasification 911 to form syngas. Supplemental sources of syngas and/or hydrogen, such as reformed natural gas or electrolyzed water, may be directed to process 900. And in certain circumstances, the entire process 900 may run on supplemental sources of synthesis gas or hydrogen as an example of gas-to-liquids conversion. In embodiments, the hydrogen and CO₂ may be purified 921. Further, syngas or any components thereof, from any source may be fed to conversion 950.

In additional embodiments, the gases produced during fermentation 910 comprise a mixture of ammonia (NH₃), CO₂, and hydrogen H₂. Recovery of fermentation gases 912 may recycle these and other gases through process 900. For example, NH₃ is recovered during a packed bed reaction with CO₂. The recovered NH₃ is converted to ammonium bicarbonate (NH₄HCO₃) for recycle to fermentation 910 (e.g. for pH control) and/or incorporated in the acids/salts stream for conversion 950. The remaining CO₂ and H₂ may purified 921 and used for conversion 950.

### Eighth Integrated Process

Referring now to FIG. 12 illustrating an embodiment of the process generally shown in FIG. 10, the process 1000 for converting biomass to hydrocarbon products, the process generally comprises the steps of fermentation 1010, withdrawing 1030 an acids/salts solution, converting 1050 the acids/salts solution to conversion products, processing 1070 the conversion products to hydrocarbon products, and recycling 1090 a portion of the by-products.

In embodiments, the process 1000 is configured similar to or substantially the same as process 900 illustrated in FIG. 11 and described herein previously. However, the process 1000 is configured to integrate fermentation 1010 with microorganisms capable of biosynthesis of hydrocarbon products. More specifically, the process 1000 is for the integration of mixotrophic organisms that convert fermentation products, including mixed acids and salts into hydrocarbon products, suitable for minimal processing 1070 and subsequent recovery. In embodiments, conversion 1050 to hydrocarbon products may be in the presence of inorganic carbon and/or energy sources. Further, the mixotrophic organisms in conversion 1050 may utilize any electron acceptor to regenerate ATP, and in non-limiting examples, oxygen, nitrates, sulfates.

Generally, in process 1000, the biomass may be pretreated 1005 prior to fermentation 1010. The biomass may be introduced in raw and/or pretreated states to a fermentor for the process of fermentation 1010. In embodiments, fermentation 1010 conditions favor the production of mixed acids and acid salts in the fermentation broth. After fermentation 1010 the fermentation broth comprising the mixed acids/salts and undigested solids is separated 1030. The unsterile liquids 1032 comprising acids/salts are removed from separation 1030 and sent to sterilization 1040. Sterilization 1040 further comprises heat exchange 1041 to recover thermal energy from heating 1042 and cooling 1043.

In embodiments, the cooled, sterilized acids/salts are directed to conversion 1050. In the present process 1000, the conversion 1050 is a mixotrophic conversion. As such, conversion 1050 follows inorganic biosynthetic conditions and CO₂, CO, and H₂ from various sources described herein, may be introduced 1051 and used during the conversion 1050. Conversion 1050 may further require cooling or heating the reaction to improve the conversion efficiency, conversion rate, reactant recovery, or optimize conditions for the microorganisms.

Conversion 1050 may use any electron acceptor known to a skilled artisan required by the chosen microorganisms accordingly. As described in multiple embodiments herein O₂, NO₃⁻, and SO₄⁻² may be suitable electron acceptors to optimize conversion 1050 conditions. Conversion 1050 comprises venting or releasing 1052 the reduced electron acceptors and waste gases. Additionally, processes or measures may be taken to avoid losing volatile reactants from the conversion 1050. In a non-limiting example, cooling and condensing 1053 the gases during venting 1052 may be suitable to recover volatile reactants. Alternatively, as sulfates are reduced to H₂S during conversion 1050 a clean-up or sulfur recovery process 1059 may be used during venting 1052. Without limitation by theory, a clean-up process or sulfur recovery process 1059 prevents release of H₂S gas to atmosphere.

The conversion 1050 forms hydrocarbons. In embodiments, the hydrocarbon products from conversion 1050 may be externalized as extracellular matrix molecules or as extracellular secretions. In alternate embodiments, the hydrocarbons from conversion 1050 are intracellular molecules. The conversion process 1050 may include selectively separating microorganisms 1054 for recycling within the conversion process 1050. In embodiments where microorganisms produce hydrocarbons as extracellular matrix molecules or extracellular secretions do not require the lysis 1060 of the microorganisms. In embodiments where the microorganisms produce intracellular hydrocarbons, the microorganisms are subjected to lysing 1060. Lysing 1060 further comprises concentrating the microorganism cell mass and any process suitable for rupturing a cell membrane. Non-limiting examples of lysing 1060 including centrifuging, osmotic shocking, supercritical fluid extraction, solvent extraction, cold pressing, shearing, homogenizing, blending, milling, sonication, or other techniques. As understood by a skilled artisan, there are many ways to recover the hydrocarbons. As hydrocarbons tend to be immiscible, they therefore float to or on the surface of aqueous solutions. In embodiments, the extracellular hydrocarbons may be decanted or skimmed and directed to processing 1070, without limitation.

Recovery 1070 may optionally comprise separation, purification, and refining of hydrocarbons from conversion 1050. In embodiments, processing 1070 may be used for cracking, upgrading, or other refinery process without limitation. As the hydrocarbons in process 1000 were directly produced by the microorganisms during conversion 1050, they may be ready for immediate sale or implementation into other process. In non-limiting examples, the hydrocarbons may be liquid fuels, solvents, or other chemical commodities.

As previously discussed in relation to process 900, process 1000 may also integrate other methods and processes. Without limitation by theory, integration of other steps, feeds, and processes into the process 1000 reduces capital cost, improves raw material usage, and improves operational efficiency and flexibility. Non-limiting process examples include gasification 1011, ammonia recovery 1012, and electricity generation 1013. In certain embodiments, the undigested residue from fermentation 1010 may be used for gasification 1011 to form syngas. Supplemental sources of syngas and/or hydrogen, such as reformed natural gas or electrolyzed water, may be directed to process 1000. And in certain circumstances, the entire process 1000 may run on supplemental sources of synthesis gas or hydrogen as an example of gas-to-liquids conversion. In embodiments, the hydrogen and CO₂ may be purified 1021. Further, syngas, or any component thereof, from any source, may be fed to conversion 1050.

In additional embodiments, the gases produced during fermentation 1010 comprise a mixture of ammonia (NH₃), CO₂, and hydrogen H₂. Recovery of NH₃ 1012 is used for conversion to ammonium bicarbonate (NH₄HCO₃) and recycle to fermentation 1010 (e.g. for pH control) and/or incorporation in the acids/salts stream for conversion 1050. The remaining CO₂ and H₂ may purified 1021 and used for conversion 1050.

### Photo-Mixotrophic Methods

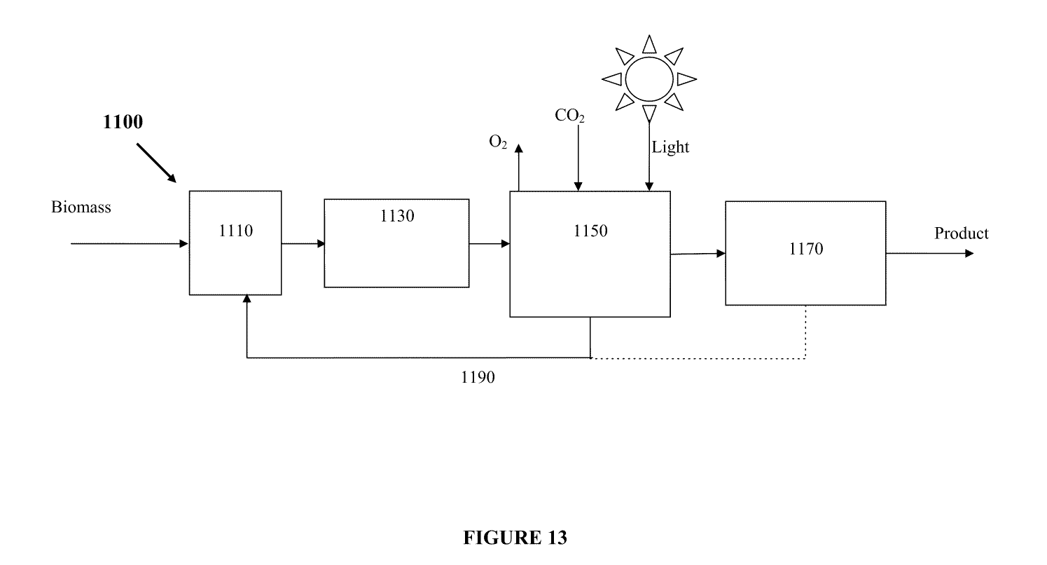

Referring now to FIG. 13, illustrating a block flow diagram for a general process 1100 for converting acids, acid salts, and combinations thereof to chemical products or hydrocarbon products. The process 1100 includes hydrocarbons, hydrocarbon-like molecules, or combinations thereof produced by photo-mixotrophic organisms. The process 1100 comprises introducing biomass to a fermenter 1110, separating liquid fermenter products 1130, converting the liquid fermenter products to form conversion products 1150, and processing the conversion products into chemical products or hydrocarbon products 1170. Some materials are recycled 1190 back through the process 1100 by re-introduction to the fermentation step 1110 from the conversion 1150 and/or processing 1170 step.

In embodiments FIG. 13 is a process flow diagram for the integration of anaerobic fermentation 1110 and photo-mixotrophic conversion 1150. Fermentation 1110 generally comprises a variety of anaerobic bacteria converting biomass into mixed acids or salts, herein acids/salts. Without limitation by theory, suitable biomass comprises any biological material that ferments to form acids or salts in solution. The resulting acid/salt solution is separated 1130 and sent to photo-mixotrophic conversion 1150. During the conversion 1150, at least one photo-mixotrophic organism converts the acids/salts solution into a hydrocarbons or hydrocarbon-like conversion products, in the presence of inorganic energy sources and light. Inorganic energy sources may comprise hydrogen, carbon dioxide, and/or other inorganic molecules. The conversion products are processed 1170 into biofuels, biochemical products, or other chemical commodities, without limitation, by heterotrophic or photoautotrophic pathways. Water, intact and lysed cells, macromolecules, byproducts, and un-reacted acids/salts from the photo-mixotrophic conversion 1150 and processing 1170 may be redirected for recycling 1190 to recover acids/salts, by-products, biofuels, biochemical, or other components. In certain instances, the photo-mixotrophic conversion step 1150 returns organic materials derived from the organisms (e.g. biomass) to the recycling step 1190 for fermentation 1110 and the processing step 1170 returns water and dilute solutions. Alternatively, only one process chosen from the conversion step 1150 and the processing step 1170 feeds the recycling step 1190.

In the following discussion and illustrations of various embodiments of the general process discussed hereinabove, similar processes and pathways are noted by similar reference numerals. For example, the step of fermentation 110 may be indicated as 210, 310, 410, etc in the subsequent figures and discussion. Additionally, the step of conversion 150 may be indicated as 250, 350, 450, etc. While the general steps may be related, the specific properties, reactions, pathways, and products of the general steps may differ, and therefore should not be limited to any particular embodiment described preceding discussion, or shown in a preceding illustration, but only by the description that accompanies it.

### Ninth Integrated Process

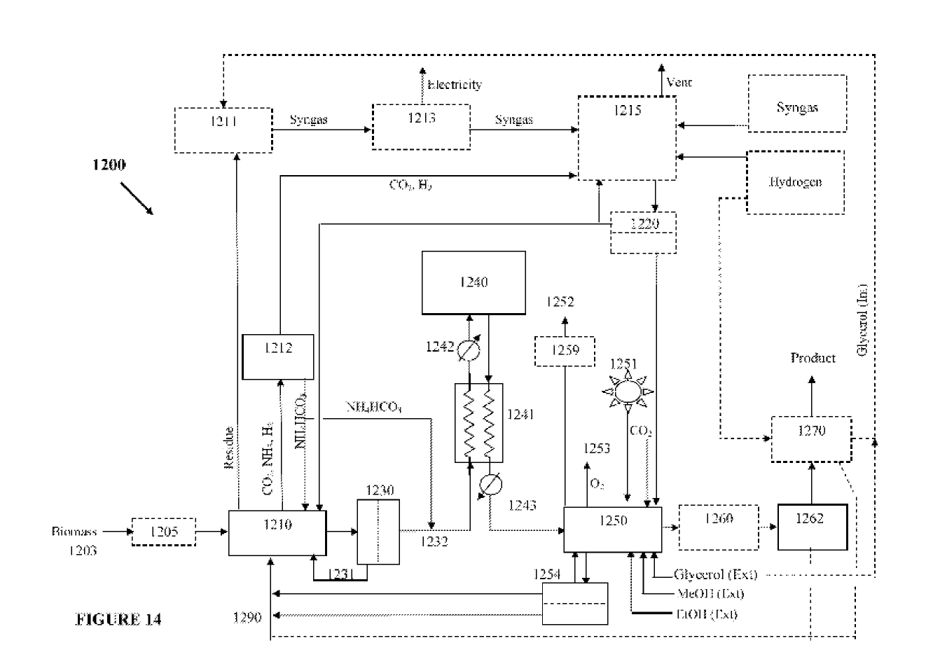

Referring now to FIG. 14 illustrating an embodiment of the process generally shown in FIG. 13, the process 1200 for converting biomass to hydrocarbon products, the process generally comprises the steps of fermentation 1210, separating 1230 an acids/salts solution, converting 1250 the acids/salts solution to conversion products, recovering 1262 the product, and, if necessary, processing 1270 the recovered conversion products to hydrocarbon products, and recycling 1290 a portion of the by-products or residues.

In embodiments, the process 1200 is configured to integrate a fermentation process 1210 with a photo-bioreactor process 1250 for biosynthesis of hydrocarbons and/or hydrocarbon-like molecules. More specifically, the process 1200 is for the integration of photo-mixotrophic organisms, including the non-limiting examples: algae, cyanobacteria (blue-green algae), euglena, and other phytoplankton. In certain instances a photo-bioreactor may comprise an algae farm, a pond, or a cultured (i.e. commercial monoculture) population of photo-mixotrophic organisms. In embodiments, photo-mixotrophic organisms are photo-autotrophic organisms that utilize light as an energy source to fix carbon photo-synthetically for biosynthetic pathways to produce hydrocarbons and/or hydrocarbon-like molecules from the mixed acids/salts. However, in the absence of light, the photo-mixotrophs function as heterotrophs to convert fermentation products, including mixed acids and salts, to produce hydrocarbons and/or hydrocarbon-like molecules. As may be understood by a person of skill in the art, photo-autotrophy may result in increased hydrocarbon and/or hydrocarbon-like molecules synthesis.

Referring again to FIG. 14, photo-mixotrophs are capable of producing large quantities of cell biomass during photosynthesis-mediated growth. In embodiments, the photo-mixotrophs used for conversion 1250 may produce cell biomass that supplements, is sufficient for, or is in excess of the needs for fermentation 1210, without limitation. In instances, the quantity of biomass from conversion 1250 may change with the conditions of process 1200, and a variable quantity of photo-mixotroph-derived biomass from conversion 1250 may be used for fermentation 1210. In embodiments, the mass volume of photo-mixotroph-derived biomass used in fermentation 1210 may range from about 0% to about 99% by weight/volume concentration of photo-mixotroph-derived biomass; alternatively, about 1% to about 100% by weight/volume concentration of photo-mixotroph-derived biomass; and alternatively between about 20% and about 75% of the biomass for fermentation 1210.

Alternatively, external biomass 1203 from sources outside of the process 1200 may be introduced to the fermentor for the process of fermentation 1210. In embodiments, biomass may comprise the non-limiting examples, municipal solids waste, farm waste, lignocellulosic/starchy crops, or combinations thereof, and the biomass is digested during fermentation 1210. Optionally, the external and/or internal biomass may be pretreated 1205 prior to fermentation 1210. In these embodiments, the external biomass has a high lignin content that is insoluble and/or interferes with the mixed-acid fermentation. Non-limiting examples of potential pretreatment processes include sulfuric acid pretreatment, hot water pretreatment, steam pretreatment or autoclaving, ammonia pretreatment, ammonia-fiber expansion (AFEX), and lime pretreatment. Pretreatment processes examples may be found for example in U.S. Pat. No. 5,865,898, U.S. Pat. No. 5,693,296, or U.S. Pat. No. 6,262,313, without limitation, incorporated herein by reference. After pretreatment 1205, the pretreated biomass is subjected to mixed acid fermentation 1210. In instances, fermentation 1210 may be fermentation 1210 or aerobic digestions. Fermentation 1210 conditions favor the production of mixed acids and acid salts in the fermentation broth.

After fermentation 1210 the fermentation broth comprising the mixed acids/salts is separated 1230. In embodiments, the fermentation broth comprises a non-sterile suspension or colloid including biomass debris, suspended solids, cellular debris, microorganisms, acids/salts and other fermentation products. In embodiments, separating 1230 the fermentation broth further comprises separating the solids from the liquids. The solids 1231 including biomass debris, macroscopic suspended solids and particles are screened, filtered, settled, centrifuged, or decanted from the unsterilized liquids including microorganisms, microscopic suspended solids, cellular debris and the acids/salts. The separated solids 1231 are returned for further digestion and fermentation 1210 to acids/salts. The non-sterile liquids 1232 comprising acids/salts are removed 1232 from separation 1230 and sent to conversion 1250.

In embodiments the unsterile liquids, comprising the acids/salts are sterilized 1240 prior to conversion 1250. The sterilization 1240 of the fermentation broth liquids comprises thermal, pressure, autoclaving, UV, and combinations thereof, to form a sterilized acids/salts broth. Further, the fermentation broth may be sterilized 1240 in a batch process. A batch process may allow a longer residence time at the sterilization temperature. Without limitation by theory, increased residence time at the sterilization temperature kills the fermentation microorganisms in the broth and degrades enzymes and other proteins that may negatively impact the conversion of the carboxylic acids/salts in sterile conversion process 1250. Alternatively, without limitation, sterilization 1240 comprises a continuous flow process, such as a plug-flow reactor in a non-limiting example. Without limitation by theory, continuous flow sterilization reduces deposition or settling of suspended solids in the sterilization apparatus.

In embodiments, the sterilization 1240 comprises elevating the temperature of the fermentation broth to above about 100° C.; alternatively, to above 110° C.; and in certain instances over about 140° C. The sterilization 1240 further comprises heating the fermentation broth with steam 1242. In certain embodiments, the fermentation broth is sterilized for at least about 3 minutes; alternatively, for at least about 5 minutes; and alternatively, for at least about 10 minutes. Alternatively, the fermentation broth is sterilized in by continuously filling a sterilization reactor, sterilizing the fermentation broth, and draining the sterilized acids/salts.

In order to conserve, reuse, or recycle thermal energy within process 1200, heat exchange 1241 between the non-sterile fermentation broth and the sterilized acids/salts may be implemented. Without limitation by theory, heat exchange 1241 warms the unsterilized broth prior to introduction of steam 1242. Warming the unsterilized broth by heat exchange 1241 reduces the volume, temperature, and pressure of the steam introduction 1242. Additionally, heat exchange 1241 at least partially cools the sterilized acids/salts prior to conversion 1250. In embodiments, the sterilized acids/salts are further cooled 1243 prior to conversion 1250 by heat exchange with water. As above, to conserve, reuse, or recycle thermal energy within process 1200, the water from cooling 1243 having been warmed by thermal energy from the sterilized acids/salts may be used for steam introduction 1242 and sterilization 1240. In embodiments, the cooled, sterilized acids/salts are directed to conversion 1250.

In process 1200, the conversion 1250 is a photo-autotrophic or heterotrophic conversion. The conversion 1250 forms hydrocarbon and/or hydrocarbon-like products such as WE, TAG, FAME, FAEE, PHAs, other hydrocarbons, and combinations thereof, as described in detail hereinabove. In further embodiments, the hydrocarbon-like products may comprise hydrocarbon alcohols (e.g. hexanol), ketones, or aldehydes, without limitation. In embodiments, the hydrocarbons and/or hydrocarbon-like products may be externalized as extracellular matrix molecules or as extracellular secretions. In alternate embodiments, the hydrocarbons and/or hydrocarbon-like products are intracellular molecules.

In embodiments during photo-autotrophic biosynthesis, CO₂ and/or other gases are introduced 1251 during conversion 1250 as carbon and/or energy sources. Light or sunlight provides the energy for ATP generation, ATP regeneration, and the biosynthesis of hydrocarbons and/or hydrocarbon-like molecules. In embodiments, waste gases including O₂ may be vented 1253. In embodiments during heterotrophic biosynthesis, the microorganism may use O₂, air, and organic molecules for biosynthetic conversion 1250 of acids/salts to hydrocarbons and/or hydrocarbon-like molecules. Further, during heterotrophic conversion 1250 the mixotrophs may use any electron acceptor known to a skilled artisan. As described in multiple embodiments herein, O₂, NO₃⁻, and SO₄⁻² may be suitable electron acceptors to optimize conversion 1250 conditions. In embodiments, waste gases may be vented 1252. Also, certain gases may be subjected to a clean-up process or a recovery process 1259 that may be used to during venting 1252 to prevent release to atmosphere. In further embodiments, the gases, electron acceptors, and their reduced forms may be reversed as the microorganism switches between photo-autotrophic and heterotrophic conversion 1250, without limitation.

In certain instances, conversion 1250 includes introducing additional reactants from external sources for conversion 1250. Non-limiting examples of additional reactants include glycerol, methanol, or ethanol. In still other embodiments, conversion 1250 comprises venting or releasing waste gases such as O₂. However, measures may be taken to avoid losing the volatile reactants in the conversion. In certain instances, cooling and condensing the gases being vented is suitable to recover volatile reactants. Conversion may further require cooling or heating the conversion reaction to improve the conversion efficiency, conversion rate, reactant recovery, or optimize conditions for the microorganisms.

The conversion process 1250 may include selectively separating microorganisms 1254 for recycling within the conversion process 1250. In embodiments, microorganisms that produce hydrocarbons and/or hydrocarbon-like molecules that are extracellular matrix molecules or extracellular excretions do not require the lysis 1260 of the microorganisms. As understood by a skilled artisan, there are many ways to recover the hydrocarbons and/or hydrocarbon-like molecules, and in instances the hydrocarbon or hydrocarbon-like molecules tend to be immiscible and therefore float to the surface of aqueous solutions. In embodiments, the extracellular hydrocarbons and/or hydrocarbon-like molecules are decanted or skimmed and directed to processing 1270, without limitation.

The remaining suspension comprising the microorganisms, unconverted acids/salts, and conversion media liquid are directed to separation 1254. Separation 1254 may comprise filtering, settling, washing, centrifuging, or other methods to separate microorganisms and other suspended from the liquid. The liquid comprises a suspension comprising unconverted acids/salts, waste products, microorganisms, and other suspended solids, without limitation. In embodiments, the liquid is recycled 1290 for fermentation 1210. Additionally, the microorganisms, and other suspended solids are also recycled 1290 for fermentation 210. The liquids may be recycled 1290 to fermentation 1210 concurrently or separately from the microorganisms, and other suspended solids. In embodiments, the microorganisms may be returned for the conversion 1250 of further sterilized acids/salts. In certain embodiments, when the microorganisms exceed the mass, density, volume, or other measurable parameter, for efficient conversion 1250 of the acids/salts, only the excess microorganisms may be subject to recycling 1290 for fermentation. Alternatively, a portion of the microorganisms may periodically be recycled 1290 for fermentation 1210.

Alternatively, in embodiments where the microorganisms produce intracellular hydrocarbon and/or hydrocarbon-like molecules, the microorganisms are subjected to lysing 1260. Lysing 1260 further comprises concentrating the microorganism cell mass for example by centrifugation or flocculation, without limitation. Lysing 1260 may comprise any process suitable for rupturing a cell membrane and solubilizing the intracellular matrix known to a skilled artisan. Non-limiting examples of lysing 1260 including centrifuging, osmotic shocking, supercritical fluid extraction, solvent extraction, cold pressing, shearing, homogenizing, blending, milling, sonication, or other techniques.

In embodiments, lysing 1260 the microorganisms comprises recovering 1262 the hydrocarbons and/or hydrocarbon-like molecules from the other cellular components, comprising proteins, enzymes, membranes, nucleic acids and liquids from the lysed microorganisms. As previously described, there are many ways to recover the hydrocarbons and/or hydrocarbon-like molecules, and in instances the hydrocarbon or hydrocarbon-like molecules tend to be immiscible and therefore float to the surface of aqueous solutions. In embodiments, the extracellular hydrocarbons and/or hydrocarbon-like molecules are decanted or skimmed and, optionally, directed to processing 1270, without limitation. Alternatively, the hydrocarbon and hydrocarbon-like molecules may be aggregated with other cellular components that are immiscible or hydrophobic. As such, to separate the hydrocarbon and/or hydrocarbon-like molecules, any process known to a person of skill in the art may be used, including membrane separation, filtering, and centrifuging. The other cellular components, comprising proteins, enzymes, membranes, and liquids are recycled 1290 for fermentation 1210. Intracellular liquids may be recycled 1290 to fermentation 1210 concurrently or separately from the other cellular components.

In embodiments, whether from extracellular production or cell lysing and recovery, the hydrocarbon and/or hydrocarbon-like molecules are directed to processing 1270. Without limitation, processing 1270 may chemically convert the hydrocarbon and/or hydrocarbon like molecules into chemicals, solvents, or hydrocarbon fuels that are compatible with the present fuel infrastructure. In the non-limiting examples the WE, TAG, FAME, FAEE, and PHAs previously discussed herein, processing may comprise transesterification (e.g. TAG), hydrogenation, decarboxylation, isomerization, cleaving, cross-linking, and other hydrocarbon reactions, such as refining, cracking, alkylating, polymerizing, and separating. The processing 1270 of the hydrocarbon and/or hydrocarbon-like molecules may further comprise incorporation of H₂.

The process 1200 may integrate other methods and processes. Without limitation by theory, integration of other steps, feeds, and processes into the process 1200 reduces capital cost, improves raw material usage, and improves operational efficiency and flexibility. Non-limiting process examples include gasification 1211, ammonia recovery 1212, and electricity generation 1213. In certain embodiments, the undigested residue from fermentation 1210 and excess glycerols, from conversion feeds (i.e. external) and conversion (i.e. internal) sources may be used for gasification 1211 to form syngas. The syngas production may be used in electricity generation 1213, as thermal energy derived from cooling the gasification products comprising syngas, may be used to generate electricity, for example to generate steam to run electrical turbines. All or a portion of the products of gasification 1211 may be used in electricity generation and/or may be passed directly to downstream processes, such as hydrogen recovery, or a chemoautotrophic process, such as described herein. Without limitation by theory, gasification of the undigested residue to syngas may refine out pollutants and/or corrosive compounds, thereby making electricity generation cleaner.

Alternatively, the syngas production 1213 may be used for other microorganism mediated processes 1215. In certain embodiments, the syngas may be converted to acids/salts by a chemoautotrophic microorganism in process 1215. The chemoautotrophic microorganism may comprise pure, mixed, natural, or genetically modified cultures. The acids/salts derived from chemoautotrophic process 1215 may be used to supplement those from fermentation 1210 for conversion 1250. Chemoautotrophic process 1215 may additionally supply biomass for fermentation 1210 in the form of waste products, microorganisms, and acids/salts from a separation process 1220.

In additional embodiments, the gases produced during fermentation 1210 comprise a mixture of NH₃, CO₂, and H₂. Recovery and redirection of fermentation gases 1212 may make these gases available throughout process 1200. For example, NH₃ is recovered during a packed bed reaction with CO₂, which converts NH₃ into ammonium bicarbonate (NH₄HCO₃) for recycle to fermentation 1210 (e.g. for pH control) and/or incorporation in the acids/salts stream as an nitrogen source for conversion 1250.

Supplemental sources of syngas and/or hydrogen, such as reformed natural gas or electrolyzed water, may be directed to process 1200. And in certain circumstances, the entire process 1200 may run on supplemental sources of synthesis gas or hydrogen as an example of photo-mixotroph mediated gas-to-liquids conversion.

### Tenth Integrated Process

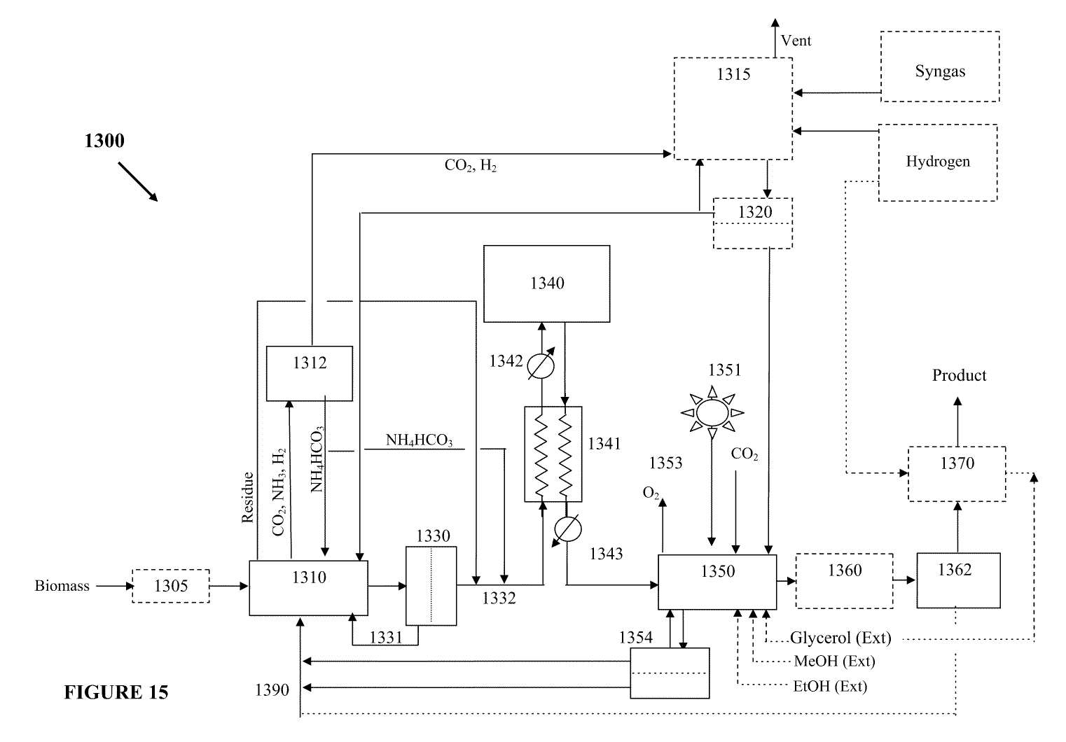

Referring now to FIG. 15 illustrating an embodiment of the process generally shown in FIG. 13, the process 1300 for converting biomass to hydrocarbon products, the process generally comprises the steps of fermentation 1310, separating 1330 an acids/salts solution and undigested residue, converting 1350 the acids/salts solution to conversion products, processing 1370 the conversion products to hydrocarbon products, and recycling 1390 a portion of the products.

Process 1300 is configured similar or substantially the same as the process 1200 as illustrated in FIG. 14, and discussed previously. However, in embodiments the process 1300 includes a redirection of undigested residues from fermentation 1310 through sterilization 1340 and conversion 1350, in contrast to use of said residue for gasification 1211 shown in FIG. 14. Biomass from conversion 1350, and/or external sources may be fed to the fermentation 1510, with optional pretreatment. In additional embodiments, the fermentation broth is not subject to separation 1330, such that the fermentation broth removal 1332 comprises an non-sterile suspension or colloid including biomass debris, suspended solids, cellular debris, microorganisms, acids/salts and other fermentation products. The non-sterile fermentation broth comprising these suspended portions is directed to sterilization 1340. In certain instances, the residues may be directed through sterilization 1340 and conversion 1350 by a separate stream, or may be used with an optional alternate separator, such that all or a portion of the solids may be recovered and recycled as needed through the process 1300.

In embodiments the non-sterile fermentation broth, comprising the suspended solids and biomaterial described, in addition to acids/salts, is sterilized 1340 prior to conversion 1350. The sterilization 1340 of the fermentation broth comprises thermal, pressure, autoclaving, UV, and combinations thereof, to form a sterilized acids/salts broth. Further, the fermentation broth may be sterilized 1340 in a batch process. A batch process may allow a longer residence time at the sterilization temperature. Without limitation by theory, increased residence time at the sterilization temperature kills the fermentation microorganisms, degrades biomaterial, proteins, enzymes, and organic molecules, and thermally degrades any suspended solids in the broth. Alternatively, without limitation, sterilization 1340 comprises a continuous flow process, such as through a plug-flow reactor, that may reduce deposition or settling of suspended solids in the sterilization apparatus. Without limitation by any particular theory, sterilization 1340 of the fermentation broth comprising these biomaterials kills microorganism that may negatively affect conversion 1350.

In further embodiments, the sterilization 1340 comprises elevating the temperature of the fermentation broth to above about 100° C.; alternatively, to above 110° C.; and in certain instances over about 150° C. The sterilization 1340 further comprises heating the fermentation broth with steam 1342. In certain embodiments, the fermentation broth is sterilized for at least about 3 minutes; alternatively, for at least about 5 minutes; and alternatively, for at least about 10 minutes. Without limitation by theory, increased temperatures and increased residence time may serve to thermally degrade the biomass debris, and suspended organic solids for conversion 1350. Alternatively, the fermentation broth is sterilized by continuously filling a sterilization reactor, sterilizing the fermentation broth, and draining the sterilized acids/salts.

In order to conserve, reuse, or recycle thermal energy within process 1300, heat exchange 1341 between the non-sterile fermentation broth and the sterilized acids/salts may be implemented. Without limitation by theory, heat exchange 1341 warms the unsterilized broth prior to introduction of steam 1342. Warming the unsterilized broth by heat exchange 1341 reduces the volume, temperature, and pressure of the steam introduction 1242. In embodiments, the cooled, sterilized fermentation broth, including the acids/salts are directed to conversion 1350.

In process 1300, the conversion 1350 is a photo-autotrophic or heterotrophic conversion. The conversion 1350 forms hydrocarbon and/or hydrocarbon-like products such as WE, TAG, FAME, FAEE, PHAs, other hydrocarbons, and combinations thereof, as described in detail hereinabove. In further embodiments, the hydrocarbon-like products may comprise hydrocarbon alcohols (e.g. hexanol), ketones, or aldehydes, without limitation. In embodiments, the hydrocarbons and/or hydrocarbon-like products may be externalized as extracellular matrix molecules or as extracellular secretions. In alternate embodiments, the hydrocarbons and/or hydrocarbon-like products are intracellular molecules.

In embodiments during photo-autotrophic biosynthesis (photosynthesis), CO₂ and/or other gases are introduced 1351 during conversion 1350 as carbon and/or energy sources. Light or sunlight provides the energy for ATP generation, ATP regeneration, and the biosynthesis of hydrocarbons and/or hydrocarbon-like molecules. In embodiments, waste gases including O₂ may be vented 1353. In embodiments during heterotrophic biosynthesis, the microorganism may use O₂, air, and the organic molecules, suspended solids, cellular and biomass debris for biosynthetic conversion 1350 of acids/salts to hydrocarbons and/or hydrocarbon-like molecules. Without limitation by theory, sterilization removes competing microorganisms and might thermally degrades the suspended solids, biomass debris, cellular debris, and other organic material. In certain instances, the sterilized fermentation broth in process 1300 may be more readily taken up and converted to hydrocarbons in conversion 1350. Further, during heterotrophic conversion 1350 the mixotrophs may use any electron acceptor known to a skilled artisan. As described in multiple embodiments herein, O₂, NO₃⁻, and SO₄⁻² may be suitable electron acceptors to optimize conversion 1350 conditions. In embodiments, waste gases may be vented 1353. Also, certain gases may be subjected to a clean-up process or a recovery process that may be used to during venting to prevent release to atmosphere. In further embodiments, the gases, electron acceptors, and their reduced forms may be reversed as the microorganism switches between photo-autotrophic and heterotrophic conversion 1350, without limitation.

In certain instances, conversion 1350 includes introducing additional reactants from external sources for conversion 1350. Non-limiting examples of additional reactants include glycerol, methanol, or ethanol. In still other embodiments, conversion 1350 comprises venting or releasing waste gases such as O₂. However, measures may be taken to avoid losing the volatile reactants in the conversion. In certain instances, cooling and condensing the gases being vented is suitable to recover volatile reactants. Conversion may further require cooling or heating the conversion reaction to improve the conversion efficiency, conversion rate, reactant recovery, or optimize conditions for the microorganisms.

The conversion process 1350 may include selectively separating microorganisms 1354 for recycling within the conversion process 1350. In embodiments, microorganisms that produce hydrocarbons and/or hydrocarbon-like molecules that are extracellular matrix molecules or extracellular excretions do not require the lysis of the microorganisms. As understood by a skilled artisan, there are many ways to recover the hydrocarbons and/or hydrocarbon-like molecules, and in instances the hydrocarbon or hydrocarbon-like molecules tend to be immiscible and therefore float to the surface of aqueous solutions. In embodiments, the extracellular hydrocarbons and/or hydrocarbon-like molecules may be decanted or skimmed and directed to processing 1370, without limitation.

Alternatively, in embodiments where the microorganisms produce intracellular hydrocarbon and/or hydrocarbon-like molecules, the microorganisms are subjected to lysing 1360. Lysing 1360 further comprises concentrating the microorganism cell mass for example by centrifugation or flocculation, without limitation. Lysing 1360 may comprise any process suitable for rupturing a cell membrane and solubilizing the intracellular matrix known to a skilled artisan. In embodiments, lysing 1360 the microorganisms comprises recovering 1362 the hydrocarbons and/or hydrocarbon-like molecules from the microorganisms and undigested fermentation residues including, cellular components, proteins, enzymes, membranes, nucleic acids, liquids, and other materials without limitation. As previously described, there are many ways to recover the hydrocarbons and/or hydrocarbon-like molecules from a mixed suspension.

The remaining fermentation residues, microorganisms, unconverted acids/salts, and conversion media liquid may be recycled 1390 for fermentation 1310. Additionally, the unconverted acids/salts, waste products, microorganisms, and other suspended solids are also recycled 1390 for fermentation 1310. Alternatively, a portion of the remaining fermentation residues, microorganisms, unconverted acids/salts, and conversion media liquid be returned to the conversion 1350 step as the biomaterial for heterotrophic conversion to further acids/salts

In embodiments, whether from extracellular production or cell lysing and recovery, the hydrocarbon and/or hydrocarbon-like molecules are directed to processing 1370. Without limitation, processing 1370 may chemically convert the acids/salts into chemicals, solvents, or hydrocarbon fuels that are compatible with the present fuel infrastructure. In non-limiting examples, for the WE, TAG, FAME, FAEE, and PHAs previously discussed herein, processing may comprise transesterification (e.g. TAG), hydrogenation, decarboxylation, isomerization, cleaving, cross-linking, and other hydrocarbon reactions, such as refining, cracking, alkylating, polymerizing, and separating. The processing 1370 of the hydrocarbon and/or hydrocarbon-like molecules may further comprise incorporation of H₂.

The process 1300 may integrate other methods and processes. Without limitation by theory, integration of other steps, feeds, and processes into the process 1300 reduces capital cost, improves raw material usage, and improves operational efficiency and flexibility. In embodiments, the gases produced during fermentation 1310 comprise a mixture of NH₃, CO₂, and H₂. Recovery and redirection of fermentation gases 1312 may make these gases available throughout process 1300. For example, NH₃ is recovered during a packed bed reaction with CO₂, which yields ammonium bicarbonate (NH₄HCO₃) for recycle to fermentation 1310 (e.g. for pH control) and/or incorporation in the acids/salts stream as nitrogen source for conversion 1350. In certain embodiments, the remaining gases may be converted to acids/salts by a chemo-autotrophic microorganism process 1315. The chemoautotrophic microorganism may comprise pure, mixed, natural, or genetically modified cultures, similar to or substantially the same as any previously described herein. The acids/salts derived from chemoautotrophic process 1315 may be used to supplement those from fermentation 1310 for conversion 1350. Chemoautotrophic process 1315 may additionally supply biomass for fermentation 1310 or conversion 1350 in the form of waste products, dead microorganisms, and acids/salts from a separation process 1320.

Supplemental sources of syngas and/or hydrogen, such as reformed natural gas or electrolyzed water, may be directed to process 1300. And in certain circumstances, the entire process 1300 may run on supplemental sources of synthesis gas or hydrogen as an example of photo-mixotroph mediated gas-to-liquids conversion.

### Eleventh Integrated Process

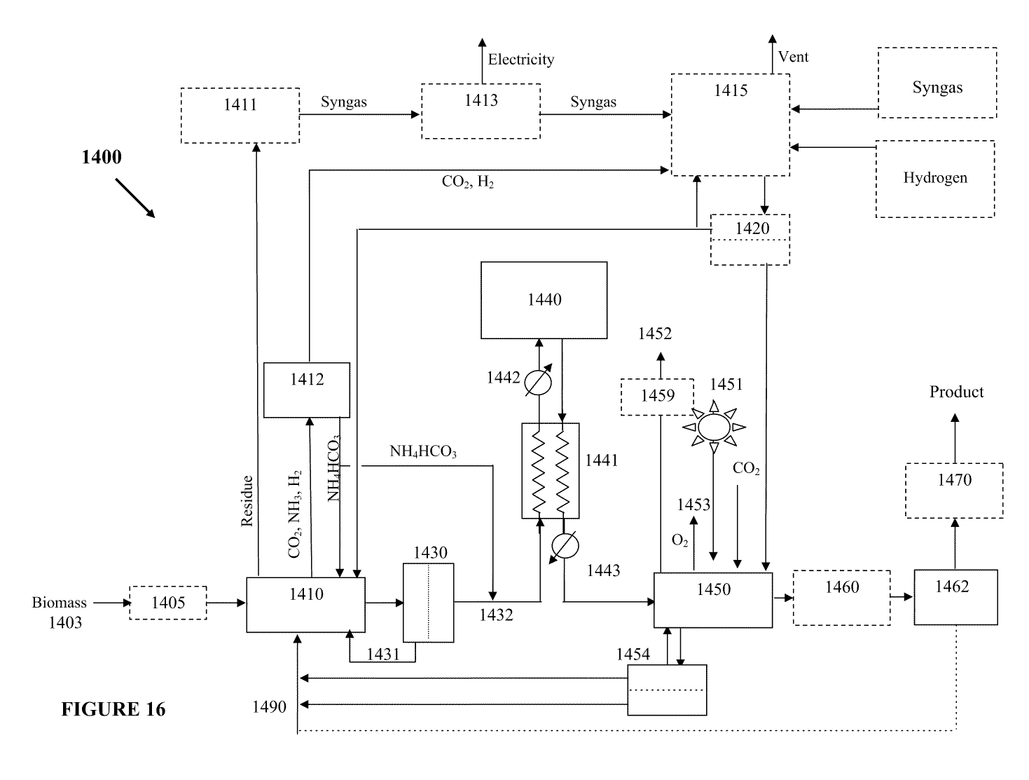

Referring now to FIG. 16 illustrating an embodiment of the process generally shown in FIG. 13, the process 1400 for converting biomass to hydrocarbon products, the process generally comprises the steps of fermentation 1410, separating 1432 an acids/salts solution and undigested biomass/residues, converting 1450 the acids/salts solution to conversion products, processing 1470 the conversion products to hydrocarbon products, and recycling 1490 a portion of the products. Further, process 1400 is configured similar to the process 1200 as illustrated in FIG. 14, and discussed previously. However, process 1400 is configured for the direct synthesis of hydrocarbons during conversion 1450. As such, the steps related to processing 1470 and recycling 1490 may be different than those found in process 1200.

Referring again to FIG. 16, photo-mixotrophs are capable of producing large quantities of biomass during photosynthesis-mediated growth. In embodiments, the photo-mixotrophs used for conversion 1450 may produce biomass that supplements, is sufficient for, or is in excess of the needs for fermentation 1410, without limitation. In embodiments, the mass volume of photo-mixotroph-derived biomass from conversion 1450 used in fermentation 1410 may range from about 0% to about 99% by weight/volume concentration of photo-mixotroph-derived biomass; alternatively, about 1% to about 100% by weight/volume concentration of photo-mixotroph-derived biomass; and alternatively between about 20% and about 75% by weight/volume concentration of photo-mixotroph-derived biomass is used for fermentation 1410.

Alternatively, external biomass 1403 from sources outside of the process 1400 may be introduced to the fermentor for the process of fermentation 1410. Optionally, the external and/or internal biomass is pretreated 1405 by any process prior to fermentation 1410. After pretreatment 1405, the pretreated biomass is subjected to mixed acid fermentation 1410. In instances, fermentation 1410 conditions favor the production of mixed acids and acid salts in the fermentation broth.

After fermentation 1410 the fermentation broth comprising the mixed acids/salts is separated 1430. In embodiments, separating 1430 the fermentation broth further comprises separating the solids from the liquids. The separated solids 1431 are returned for further digestion and fermentation 1410 to acids/salts. The separated unsterile liquids 1432 comprising acids/salts are removed from separation 1430. In embodiments the unsterile liquids 1432, comprising the acids/salts are sterilized 1440 by any process. In embodiments, the sterilization 1440 comprises elevating the temperature of the fermentation broth to above about 100° C.; alternatively, to above 110° C.; and in certain instances over about 140° C. The sterilization 1240 further comprises heating the fermentation broth with steam 1442. In certain embodiments, the fermentation broth is sterilized for at least about 3 minutes; alternatively, for at least about 5 minutes; and alternatively, for at least about 10 minutes. Alternatively, the fermentation broth is sterilized in by continuously filling a sterilization reactor, sterilizing the fermentation broth, and draining the sterilized acids/salts. In order to conserve, reuse, or recycle thermal energy within process 1400, heat exchange 1441 between the unsterile fermentation broth, the sterilized acids/salts, the steam 1442 and sterilized brother cooling 1443 may be implemented as previously described herein.

Conversion 1450 is a photoautotrophic or heterotrophic conversion. The conversion 1450 forms hydrocarbons. In embodiments during photo-autotrophic biosynthesis, CO₂ and/or other gases are introduced 1451 during conversion 1450 as carbon and/or energy sources. Light or sunlight provides the energy for ATP generation, ATP regeneration, and the biosynthesis of hydrocarbons. In embodiments, waste gases including O₂ may be vented 1453. In other embodiments, during heterotrophic biosynthesis, the microorganism may use O₂, air, and organic molecules for biosynthetic conversion 1450 of acids/salts to hydrocarbons. Further, during heterotrophic conversion 1450 the mixotrophs may use any electron acceptor known to a skilled artisan. As described in multiple embodiments herein, O₂, NO₃⁻, and SO₄⁻² may be suitable electron acceptors for conversion 1450 conditions. In embodiments, waste gases may be vented 1453. Also, certain gases may be subjected to a clean-up process or a recovery process 1459 that may be used to during venting 1452 to prevent release to atmosphere. In further embodiments, the conversion 1450 requirements for gases, electron acceptors, and their reduced forms may be reversed as the microorganism switches between photo-autotrophic and heterotrophic conversion 1450, without limitation.

In certain instances, conversion 1450 may include introducing additional reactants from external sources for conversion 1450, such as glycerol, methanol, or ethanol, for example as shown in FIG. 15. In still other embodiments, conversion 1450 comprises venting or releasing waste gases such as O₂. However, measures may be taken to avoid losing the volatile reactants in the conversion. In certain instances, cooling and condensing the gases being vented is suitable to recover volatile reactants. Conversion 1450 may further require cooling or heating the conversion reaction to improve the conversion efficiency, conversion rate, reactant recovery, or optimize conditions for the microorganisms.

In embodiments, the hydrocarbons may be externalized as extracellular matrix molecules or as extracellular excretions. In alternate embodiments, the hydrocarbons are intracellular molecules. The conversion process 1450 may include selectively separating microorganisms 1454 for recycling within the conversion process 1450. In embodiments, microorganisms that produce hydrocarbons that are extracellular matrix molecules or extracellular excretions do not require the lysis 1460 of the microorganisms. Alternatively, in embodiments where the microorganisms produce intracellular hydrocarbons, the microorganisms are subjected to lysing 1460. In embodiments, lysing 1460 the microorganisms comprises separating 1462 the hydrocarbons from the other lysed cellular components. As previously described, there are many ways to recover the hydrocarbons and direct them to processing 1470. In embodiments, the remaining conversion materials may be recycled 1490 for additional fermentation 1410.

Recovering 1462 comprises separation, purification, and refining of hydrocarbons from conversion 1450. In embodiments, processing 1470 may be used for cracking, upgrading, or other refinery process without limitation. As the hydrocarbons in process 1400 were directly produced by the microorganisms during conversion 1450, they may be ready for immediate sale or implementation into other process. In non-limiting examples, the hydrocarbons may be liquid fuels, solvents, or other chemical commodities.

The process 1400 may integrate other methods and processes, including the non-limiting examples gasification 1411, ammonia recovery 1412, and chemoautotrophic conversion 1415. Further, the process 1400 may directly or indirectly supplement the production of electricity 1413 by the formation of syngas, hydrogen, and the recovery of thermal energy therefrom. In embodiments, the integrated methods and processes may be used to recover thermal energy or produce electricity for use throughout process 1400. The integrated methods and processes may be directed to the production of H₂ and/or syngas for use throughout the process as previously described. Alternatively, supplemental sources of syngas and/or hydrogen, such as reformed natural gas or electrolyzed water, may be directed to process 1400. And in certain circumstances, the entire process 1400 may run on supplemental sources of synthesis gas or hydrogen as an example of photo-mixotroph mediated gas-to-liquids conversion.

### Twelfth Integrated Process

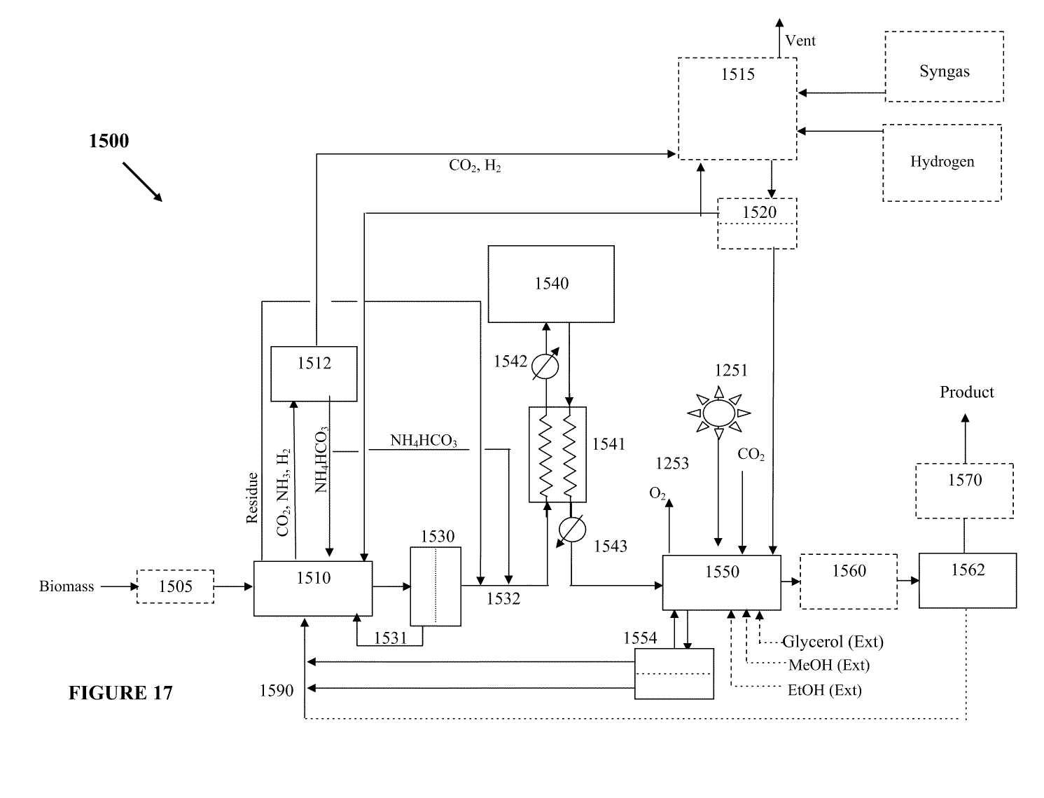

Referring now to FIG. 17 illustrating an embodiment of the process generally shown in FIG. 13, the process 1500 for converting biomass to hydrocarbon products, the process generally comprises the steps of fermentation 1510, separating 1530 an acids/salts solution, converting 1550 the acids/salts solution to conversion products, processing 1590 the conversion products to hydrocarbon products 1570, and recycling a portion of the products. Further, process 1500 is configured similarly to the process 1300 as illustrated in FIG. 15, and discussed previously. However, process 1500 is configured for the direct synthesis of hydrocarbons during conversion 1550. As such, the steps related to processing 1570 and recycling 1590 may be different than those found in process 1300.

In embodiments process 1500 includes a redirection of undigested residues from fermentation 1510 through sterilization 1540 and conversion 1550. Biomass from conversion 1550 and/or external sources may be fed to fermentation 1510, with optional pretreatment. In additional embodiments, the fermentation broth is not subject to filtering 1530, such that the fermentation broth removal 1532 comprises an unsterile suspension or colloid including biomass debris, suspended solids, cellular debris, microorganisms, acids/salts and other fermentation products. The non-sterile fermentation broth comprising these suspended portions is directed to sterilization 1540. In certain instances, the residues may be directed through sterilization 1340 and conversion 1350 by a separate stream, or may be used with an optional alternate separator, such that all or a portion of the solids may be recovered and recycled as needed through the process 1300.

In embodiments the non-sterile fermentation broth, comprising the suspended solids and biomaterial described, in addition to acids/salts, is sterilized 1540 prior to conversion 1550. The sterilization 1540 of the fermentation broth comprises thermal, pressure, autoclaving, UV, and combinations thereof, to form a sterilized acids/salts broth. Further, the fermentation broth may be sterilized 1540 in a batch process. A batch process may allow a longer residence time at the sterilization temperature. Without limitation by theory, increased residence time at the sterilization temperature completely kills the fermentation microorganisms, degrades biomaterial, proteins, enzymes, and organic molecules, and may thermally degrade any suspended solids in the broth. Alternatively, without limitation, sterilization 1540 comprises a continuous flow process, such as through a plug-flow reactor, that may reduce deposition or settling of suspended solids in the sterilization apparatus. Without limitation by any particular theory, sterilization 1540 of the fermentation broth kills microorganisms that might negatively affect conversion 1550.

In further embodiments, the sterilization 1540 comprises elevating the temperature of the fermentation broth to above about 100° C.; alternatively, to above 110° C.; and in certain instances over about 140° C. The sterilization 1540 further comprises heating the fermentation broth with steam 1542. In certain embodiments, the fermentation broth is sterilized for at least about 3 minutes; alternatively, for at least about 5 minutes; and alternatively, for at least about 10 minutes. Without limitation by theory, increased temperatures and increased residence time may serve to thermally degrade the biomass debris, and suspended organic solids for conversion 1550. Alternatively, the fermentation broth is sterilized by continuously filling a sterilization reactor, sterilizing the fermentation broth, and draining the sterilized acids/salts. In order to conserve, reuse, or recycle thermal energy within process 1500, heat exchange 1541 between the non-sterile fermentation broth, the sterilized broth comprising acids/salts, the stream 1542 introduction, the steam process 1342 and the sterilized broth cooling process may be implemented. In embodiments, the cooled, sterilized fermentation broth, including the acids/salts are directed to conversion 1550.

In process 1500, the conversion 1550 is a photo-autotrophic or heterotrophic conversion that produces hydrocarbons. In embodiments during photo-autotrophic biosynthesis, CO₂ and/or other gases are introduced 1551 during conversion 1550 as carbon and/or energy sources. Light or sunlight provides the energy for ATP generation, ATP regeneration, and the biosynthesis of hydrocarbons. In embodiments, waste gases including O₂ may be vented 1553. In embodiments during heterotrophic biosynthesis, the microorganism may use O₂, air, and the organic molecules, suspended solids, cellular and biomass debris for biosynthetic conversion 1550 of acids/salts to hydrocarbons. Without limitation by theory, sterilization removes competing microorganisms and may thermally degrade the suspended solids, biomass debris, cellular debris, and other organic material. In certain instances, the sterilized fermentation broth in process 1500 comprising the degraded organic material may be more readily taken up for conversion to hydrocarbons. Further, during heterotrophic conversion 1550 the mixotrophs may use any electron acceptor known to a skilled artisan. As described in multiple embodiments herein, O₂, NO₃⁻, and SO₄⁻² may be suitable electron acceptors to optimize conversion 1550 conditions.

In further embodiments, the gases, electron acceptors, and their reduced forms may be reversed as the microorganism switches between photo-autotrophic and heterotrophic conversion 1550, without limitation. Conversion 1550 may further require cooling or heating the conversion reaction to improve the conversion efficiency, conversion rate, reactant recovery, or optimize conditions for the microorganisms.

In certain instances, conversion 1550 includes introducing additional reactants from external sources for conversion 1550. Non-limiting examples of additional reactants include glycerol, methanol, or ethanol. In still other embodiments, conversion 1550 comprises venting or releasing waste gases such as O₂. However, measures may be taken to avoid losing the volatile reactants in the conversion. In certain instances, cooling and condensing the gases being vented is suitable to recover volatile reactants. Also, certain gases may be subjected to a clean-up process or a recovery process that may be used to during venting 1553 to prevent release to atmosphere.

The conversion process 1550 may include selectively separating microorganisms 1554 for recycling within the conversion process 1550. In embodiments, microorganisms that produce hydrocarbons and/or hydrocarbon-like molecules that are extracellular matrix molecules or extracellular secretions do not require the lysis of the microorganisms. As understood by a skilled artisan, there are many ways to recover the hydrocarbons 1562, and in instances the hydrocarbons tend to be immiscible and therefore float to the surface of aqueous solutions. In embodiments, the extracellular hydrocarbons may be decanted or skimmed and directed to processing 1570, without limitation.

Alternatively, in embodiments where the microorganisms produce intracellular hydrocarbon, the microorganisms are subjected to lysing 1560. Lysing 1560 may comprise any process suitable for rupturing a cell membrane and solubilizing the intracellular matrix known to a skilled artisan. In embodiments, lysing 1560 the microorganisms comprises separating 1562 the hydrocarbons from the microorganisms and undigested fermentation residues including, cellular components, proteins, enzymes, membranes, nucleic acids, liquids, and other materials without limitation. As previously described, there are many ways to recover the hydrocarbons from a mixed suspension.

The remaining fermentation residues, microorganisms, unconverted acids/salts, waste products, dead microorganisms, and other suspended solids in the conversion liquid may be recycled 1590 for fermentation 1510. Alternatively, a portion of the remaining fermentation residues, microorganisms, unconverted acids/salts, and conversion media liquid may be kept in conversion 1550 step to maintain a healthy population of microorganisms in 1550, when microorganisms are recycled, or as the biomaterial for heterotrophic conversion to further acids/salts

Recovery 1562 comprises separation, purification, and refining of hydrocarbons from conversion 1550. On the other hand, in embodiments, processing 1570 may be used for cracking, upgrading, or other refinery process without limitation if necessary. As the hydrocarbons in process 1500 were directly produced by the microorganisms during conversion 1550, they may be ready for immediate sale or implementation into other process without much more processing or modification. In non-limiting examples, the hydrocarbons may be liquid fuels, solvents, or other chemical commodities.

The process 1500 may integrate other methods and processes. Without limitation by theory, integration of other steps, feeds, and processes into the process 1500 reduces capital cost, improves raw material usage, and improves operational efficiency and flexibility. In embodiments, the gases produced during fermentation 1510 comprise a mixture of NH₃, CO₂, and H₂. Recovery and redirection of fermentation gases 1512 may make these gases available throughout process 1500. For example, NH₃ is recovered during a packed bed reaction with CO₂, which produces ammonium bicarbonate (NH₄HCO₃) for recycle to fermentation 1510 (e.g. for pH control) and/or incorporation in the acids/salts stream for conversion 1550 as a nitrogen source. In certain embodiments, the remaining gases may be converted to acids/salts by a chemoautotrophic microorganism process 1515. The chemoautotrophic microorganism may comprise pure, mixed, natural, or genetically modified cultures, similar to or substantially the same as any previously described herein. The acids/salts derived from chemoautotrophic process 1515 may be used to supplement those from fermentation 1510 for conversion 1550. Chemoautotrophic process 1515 may additionally supply biomass for fermentation 1510 or conversion 1550 in the form of waste products, dead microorganisms, and acids/salts from a separation process 1520.

Supplemental sources of syngas and/or hydrogen, such as reformed natural gas or electrolyzed water, may be directed to process 1500. And in certain circumstances, the entire process 1500 may run on supplemental sources of synthesis gas or hydrogen as an example of photo-mixotroph mediated gas-to-liquids conversion.

### Advantages of Two Step Integration

The following discussion relates to the potential advantages that integration of a mixed acid fermentation with a microorganism mediated conversion to hydrocarbons and/or hydrocarbon-like molecules. In instances, the steps of fermentation and microorganism-mediated product of hydrocarbons have certain advantages on their own. Combining these processes into a single unit, and including other process (e.g. gasification), presents a novel path to hydrocarbons, fuels, and other commodity chemicals.

With respect to fermentation, the mixed cultures of microorganisms may be found naturally. Additionally, the populations of these aerobic, or more likely, anaerobic microorganisms are extremely diverse. The diversity provides an improvement opportunity as herein, for use into a biomass fermentor. Specifically, the diversity in microorganisms provides a broad range of viable material for fermentation of anything that biodegrades anaerobically (e.g., proteins, pectin, fats, cellulose, free sugars, etc.) into acid and/or acid salt products. Further, the diversity in organisms does not require sterility for fermentation, reducing costs for dealing with solid biomass. Additionally, the acids and/or acid salt products are removed from the fermentation as an aqueous product. Without limitation by theory, aqueous or liquid products are considerably easier to sterilize than solid biomass and later, isolate hydrocarbonaceous products therefrom.

With respect to the microorganism mediated conversion of acids/salts to hydrocarbons and hydrocarbon/like molecules, the capacity for dilute acids/salts uptake allows fermentation to remain aqueous. As the fermentation remains aqueous and is more easily sterilized, the capacity to maintain pure cultures increases the potential for genetic engineering, molecular biology, and synthetic biology to alter or improve the rate of uptake, conversion, and production of hydrocarbonaceous products for the microorganism. This represents a further avenue to improve efficiency of the two-step system. Additionally, the resulting hydrocarbon or hydrocarbon-like molecules can be recovered easily from the dilute aqueous solution by separating cells that contain the hydrocarbon or hydrocarbon-like molecules or, secreting the hydrocarbon or hydrocarbon-like molecules into the aqueous media such that they may be skimmed from the surface.

Additionally, the two step process allows for a mixotrophic microorganism, such that chemo-autotrophic or photo-autotrophic and heterotrophic growth may be capitalized. In the case of mixotrophs that can perform photo-autotrophy in addition to heterotrophy, this would reduce or minimize the size and the capital investment in photo-bioreactors, such as ponds, aquariums, aquatic greenhouses, and hydroponics, without limitation. As such, the reactors may be deeper and/or the cell density higher, because light requirements are reduced, without sacrificing high yields of hydrocarbonaceous products produced by the microorganisms. In instances, this may represent an increase in efficiency related to the increase in final yield of fuel per biomass volume and/or mass.

### CONCLUSION

In conclusion the present disclosure relates to a method, comprising: fermenting biomass to fermentation products; converting the fermentation products to hydrocarbon-like molecules biologically; and processing the hydrocarbon-like molecules. The method further comprising processing the hydrocarbon-like molecules to chemical products. And, wherein converting the fermentation products to hydrocarbon-like molecules comprises producing hydrocarbons. The method of fermenting biomass comprises mixed-acid fermentation and producing a dilute solution, wherein the dilute solution comprises acids and salts of acids from biomass solids. Additionally, the method, wherein converting the fermentation products comprises sterilizing the fermentation products, comprising introducing fermentation products to at least one microorganism chosen from the group consisting of heterotrophic microorganisms, chemo-mixotrophic organisms photo-mixotrophic microorganisms, chemo-autotrophic microorganisms, and combinations thereof. The method of the disclosure, wherein introducing fermentation products to heterotrophic organisms to at least one microorganism further comprises mixing an oxidant with the fermentation products, said oxidant chosen from the group consisting of oxygen, nitrates, sulfates, air, and combinations thereof. Further, converting the fermentation products comprises producing extracellular hydrocarbon-like molecules, producing intracellular hydrocarbon-like molecules, or combinations thereof. The hydrocarbon-like products comprise at least one product selected from the group consisting of waxy esters, triacylglycerides, triacylglycerols fatty acid methyl-esters, fatty acid ethyl-esters, poly-hydroxyalkanoates, hydrocarbons, and combinations thereof. Further, according to the disclosure converting the fermentation products to hydrocarbon-like molecules comprises producing hydrocarbons. The method wherein processing hydrocarbon-like molecules comprises isolating the hydrocarbon-like molecules; wherein isolating the hydrocarbon-like molecules comprises lysing microorganisms. The method wherein isolating the hydrocarbon-like molecules comprises separating hydrocarbon-like molecules from other fermentation products. The method wherein processing the hydrocarbon-like molecules comprises producing hydrocarbon liquids, with from about 5 carbons to about 50 carbons. Further the method comprises processing the hydrocarbon-like molecules with at least one method chosen from the group consisting of transesterifying, hydrogenating, decarboxylating, alkylating, isomerizing, polymerizing, oligomerizing, condensing, separating, cleaving, cross-linking, cracking, refining and combinations thereof. The method wherein producing hydrocarbon liquids further comprises producing at least one product chosen from the group consisting of gasoline, aviation gasoline, diesel, biodiesel, kerosene, jet fuel, solvents, lubricants, olefins, alkylolefins, commodity chemicals, and combinations thereof. The method wherein fermenting biomass to produce fermentation products further comprises gasifying undigested fermentation residues; and comprises producing syngas. The method wherein gasifying undigested fermentation residues comprises feeding gasification components to a bioreactor, wherein feeding gasification components to a bioreactor comprises feeding a chemo-autotrophic microorganism. Further according to the disclosure feeding a chemo-autotrophic microorganism comprises introducing syngas from supplemental sources. The method, wherein feeding gasification components to a bioreactor further comprises producing fermentation products for converting to hydrocarbon-like molecules. The method wherein converting fermentation products to hydrocarbon-like molecules further comprises converting supplemental alcohols. The wherein converting fermentation products to hydrocarbon-like molecules further comprises recycling remaining fermentation products to a fermenter. Wherein fermenting biomass to fermentation products further comprises producing ammonia, wherein producing ammonia comprises converting ammonia to ammonium bicarbonate. The method of wherein converting ammonia to ammonium bicarbonate comprises producing a fermentation product salt.

The present disclosure further relates to a hydrocarbon production process comprising fermenting biomass to mixed-acid fermentation products and biologically converting the fermentation products to hydrocarbon-like molecules. The process of the present disclosure further comprising processing the hydrocarbon-like molecules to chemical products. The process wherein converting the fermentation products to hydrocarbon-like molecules comprises producing hydrocarbons. Further, fermenting biomass comprises anaerobic fermentation to a dilute solution of acids and salts of acids. The process comprises separating the dilute solution from biomass solids. The process wherein separating the dilute solution further comprises recycling the biomass solids for further fermenting. The process wherein converting the fermentation products further comprises introducing fermentation products to at least one microorganism chosen from the group consisting of heterotrophic microorganisms, chemo-mixotrophic organisms, photo-mixotrophic microorganisms, chemo-autotrophic microorganisms, and combinations thereof. The process of claim 38, wherein introducing fermentation products to organisms further comprises sterilizing the fermentation products, mixing at least one gas with the fermentation products, said at least one gas selected from the group consisting of hydrogen, oxygen, nitrates, sulfates, air, carbon dioxide, carbon monoxide, and combinations thereof, and mixing at least one supplemental alcohol chosen from the group consisting of methanol, ethanol, glycerol, and combinations thereof. Also, converting the fermentation products comprises producing extracellular hydrocarbon-like molecules. Further, converting the fermentation products comprises producing intracellular hydrocarbon-like molecules. The process wherein hydrocarbon-like products comprise at least one product chosen from the group consisting of waxy esters, triacylglycerides, triacylglycerols fatty acid methyl-esters, fatty acid ethyl-esters, poly-hydroxyalkanoates, hydrocarbons, and combinations thereof. The process wherein converting the fermentation products to hydrocarbon-like molecules comprises producing hydrocarbons. The process wherein processing hydrocarbon-like molecules comprises isolating the hydrocarbon-like molecules from other fermentation products. The process wherein isolating the hydrocarbon-like molecules comprises lysing microorganisms. Further, the process wherein processing the hydrocarbon-like molecules comprises producing hydrocarbon liquids further comprises producing at least one product chosen from the group consisting of gasoline, aviation gasoline, diesel, biodiesel, kerosene, jet fuel, solvents, lubricants, olefins, alkylolefins, commodity chemicals, and combinations thereof. The process, wherein producing hydrocarbon liquids comprises producing hydrocarbons with between about 5 carbons and about 50 carbons and also, wherein producing hydrocarbon liquids further comprises at least one process chosen from the group consisting of transesterifying, hydrogenating, decarboxylating, alkylating, isomerizing, polymerizing, oligomerizing, condensing, separating, cleaving, cross-linking, cracking, refining and combinations thereof. The process wherein fermenting biomass to produce fermentation products further comprises gasifying undigested fermentation residues to syngas. The process wherein gasifying undigested fermentation residues to syngas, further comprises a water-gas shift reaction. Further, according to disclosure, the process wherein gasifying undigested fermentation residues to syngas comprises producing electricity. The process wherein gasifying undigested fermentation residues to syngas further comprises purifying hydrogen and directing the hydrogen for converting fermentation products to hydrocarbon-like molecules or hydrocarbons and wherein purifying hydrogen comprises purifying hydrogen from a supplemental hydrogen source.

A hydrocarbon-fuel production process, comprising fermenting biomass to acid/salt fermentation products, and converting acid/salt fermentation products to hydrocarbon molecules. The process wherein converting the acid/salt fermentation products comprises producing extracellular hydrocarbon-like molecules. The process wherein converting the acid/salt fermentation products comprises producing intracellular hydrocarbon-like molecules. The process further comprising processing the hydrocarbon molecules to produce a hydrocarbon fuel chosen from the group consisting of gasoline, aviation gasoline, diesel, biodiesel, jet fuel, kerosene. The process wherein fermenting biomass to acid/salt fermentation products comprises anaerobic fermenting to a dilute solution and separating solids from the dilute solution. Also, the process wherein converting the fermentation products comprises introducing fermentation products to at least one microorganism chosen from the group consisting of heterotrophic microorganisms, photo-mixotrophic microorganism, chemo-autotrophic microorganisms, and combinations thereof. The process wherein introducing fermentation products to at least one organism further comprises sterilizing the fermentation products, mixing at least one reactant gas with the fermentation products, said gas chosen from the group consisting of hydrogen, oxygen, nitrates, sulfates, air, carbon dioxide, carbon monoxide, light, and combinations thereof, and mixing at least one supplemental alcohol with the fermentation products, said alcohol chosen from the group consisting of methanol, ethanol, glycerol, and combinations thereof. The process wherein converting the fermentation products comprises producing extracellular hydrocarbon-like molecules or producing intracellular hydrocarbon-like molecules and wherein hydrocarbon-like products further comprise at least one product chosen from the group consisting of waxy esters, triacylglycerides, triacylglycerols fatty acid methyl-esters, fatty acid ethyl-esters, poly-hydroxyalkanoates, hydrocarbons, and combinations thereof. The process wherein converting the fermentation products to hydrocarbons comprises biologically producing hydrocarbons and wherein biologically producing hydrocarbons comprises isolating hydrocarbon liquids. The process wherein isolating the hydrocarbon molecules comprises lysing microorganisms to form a hydrocarbon liquid with hydrocarbons with between about 5 carbons and about 50 carbons by a process chosen from the group consisting of transesterifying, hydrogenating, decarboxylating, isomerizing, cleaving, cross-linking, refining, cracking, polymerizing, separating, cleaving, and combinations thereof.

At least one embodiment is disclosed and variations, combinations, and/or modifications of the embodiment(s) and/or features of the embodiment(s) made by a person having ordinary skill in the art are within the scope of the disclosure. Alternative embodiments that result from combining, integrating, and/or omitting features of the embodiment(s) are also within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, Rₗ, and an upper limit, Rᵤ, is disclosed, any number falling within the range is specifically disclosed. In particular, the following numbers within the range are specifically disclosed: R═Rₗ+k*(Rᵤ−Rₗ), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent . . . 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim means that the element is required, or alternatively, the element is not required, both alternatives being within the scope of the claim. Use of broader terms such as “comprises”, “includes”, and “having” should be understood to provide support for narrower terms such as “consisting of”, “consisting essentially of”, and “comprised substantially of”. Accordingly, the scope of protection is not limited by the description set out above but is defined by the claims that follow, that scope including all equivalents of the subject matter of the claims. Each and every claim is incorporated as further disclosure into the specification and the claims are embodiment(s) of the present invention. The discussion of a reference in the disclosure is not an admission that it is prior art, especially any reference that has a publication date after the priority date of this application. The disclosure of all patents, patent applications, and publications cited in the disclosure are hereby incorporated by reference, to the extent that they provide exemplary, procedural or other details supplementary to the disclosure.

We claim:

1. A method, comprising:
fermenting biomass to fermentation products;
converting the fermentation products to hydrocarbon-like molecules biologically; and
processing the hydrocarbon-like molecules.

2. The method of claim 1, further comprising processing the hydrocarbon-like molecules to chemical products.

3. The method of claim 1, wherein fermenting biomass comprises mixed-acid fermentation.

4. The method of claim 1, wherein converting the fermentation products comprises sterilizing the fermentation products.

5. The method of claim 1, wherein converting the fermentation products comprises introducing fermentation products to at least one microorganism chosen from the group consisting of heterotrophic microorganisms, chemo-mixotrophic organisms photo-mixotrophic microorganisms, chemo-autotrophic microorganisms, and combinations thereof.

6. The method of claim 5, wherein introducing fermentation products to heterotrophic organisms to at least one microorganism further comprises mixing an oxidant with the fermentation products, said oxidant chosen from the group consisting of oxygen, nitrates, sulfates, air, and combinations thereof.

7. The method of claim 5, wherein converting the fermentation products comprises producing extracellular hydrocarbon-like molecules, intracellular hydrocarbon-like molecules, or combinations thereof.

8. The method of claim 5, wherein the hydrocarbon-like products comprise at least one product selected from the group consisting of waxy esters, triacylglycerides, triacylglycerols fatty acid methyl-esters, fatty acid ethyl-esters, poly-hydroxyalkanoates, hydrocarbons, and combinations thereof.

9. The method of claim 5, wherein converting the fermentation products to hydrocarbon-like molecules comprises producing hydrocarbons.

10. The method of claim 1, wherein processing hydrocarbon-like molecules comprises isolating the hydrocarbon-like molecules.

11. The method of claim 10, wherein isolating the hydrocarbon-like molecules comprises lysing microorganisms.

12. The method of claim 10, wherein isolating the hydrocarbon-like molecules comprises separating hydrocarbon-like molecules from other fermentation products.

13. The method of claim 1, wherein processing the hydrocarbon-like molecules comprises producing hydrocarbon liquids.

14. The method of claim 1, wherein fermenting biomass to produce fermentation products further comprises gasifying undigested fermentation residues.

15. The method of claim 14, wherein gasifying undigested fermentation residues comprises producing syngas.

16. The method of claim 15, wherein gasifying undigested fermentation residues comprises feeding gasification components to a bioreactor.

17. The method of claim 16, wherein gasification components to a bioreactor further comprises producing fermentation products for converting to hydrocarbon-like molecules.

18. The method of claim 16, wherein feeding gasification components to a bioreactor comprises feeding a chemo-autotrophic microorganism.

19. The method of claim 18, wherein feeding a chemo-autotrophic microorganism comprises introducing syngas from supplemental sources.

20. The method of claim 1, wherein converting fermentation products to hydrocarbon-like molecules further comprises converting supplemental alcohols.

21. The method of claim 1, wherein converting fermentation products to hydrocarbon-like molecules further comprises recycling remaining fermentation products to a fermenter.

22. The method of claim 1, wherein fermenting biomass to fermentation products further comprises producing ammonia.

23. A hydrocarbon production process, comprising:
fermenting biomass to mixed-acid fermentation products; and
biologically converting the fermentation products to hydrocarbon molecules.

24. The process of claim 24, wherein fermenting biomass to mixed fermentation products comprises anaerobic fermenting to a dilute solution and separating solids from the dilute solution.

25. The process of claim 24, wherein converting the fermentation products further comprises introducing fermentation products to at least one microorganism chosen from the group consisting of heterotrophic microorganisms, photo-mixotrophic microorganisms, chemo-autotrophic microorganisms, and combinations thereof.

26. The process of claim 25, wherein introducing fermentation products to at least one organism further comprises:
sterilizing the fermentation products;
mixing at least one reactant gas with the fermentation products, said gas chosen from the group consisting of hydrogen, oxygen, nitrates, sulfates, air, carbon dioxide, carbon monoxide, light, and combinations thereof; and
mixing at least one supplemental alcohol with the fermentation products, said alcohol chosen from the group consisting of methanol, ethanol, glycerol, and combinations thereof.

27. The process of claim 23, wherein biologically converting the fermentation products comprises producing extracellular hydrocarbon molecules, producing intracellular hydrocarbon molecules, or combinations thereof.

28. The process of claim 23, further comprising producing hydrocarbons with between about 5 carbons and about 50 carbons by a process chosen from the group consisting of transesterifying, hydrogenating, decarboxylating, isomerizing, cleaving, cross-linking, refining, cracking, polymerizing, separating, cleaving, and combinations thereof.

29. The method of claim 23, further comprising
gasifying undigested fermentation residues to produce gasification components;
mixing at least a portion of the gasification components with the fermentation products for converting to hydrocarbons; and
directly at least a portion of the gasification components to at least one additional process.

30. A hydrocarbon-fuel production process, comprising:
fermenting biomass to acid/salt fermentation products; and
converting acid/salt fermentation products to form a biofuel.
